(19) United States
(12) Patent Application Publication
(10) Pub. No.: US 2020/0171165 A1
(43) Pub. Date: Jun. 4, 2020

(54) ENHANCED TARGETED DELIVERY OF THERAPEUTIC AGENTS

(71) Applicant: Proteogenomics Research Institute for Systems Medicine, La Jolla, CA (US)

(72) Inventors: Jan E. SCHNITZER, Encinitas, CA (US); Philip Sung-Whan OH, San Diego, CA (US); Adrian CHRASTINA, Chula Vista, CA (US); Michael D. LEVIN, San Diego, CA (US); Bogdan Z. OLENYUK, Poway, CA (US); Rajesh KOTA, San Diego, CA (US)

(73) Assignee: Proteogenomics Research Institute for Systems Medicine, La Jolla, CA (US)

(21) Appl. No.: 16/699,455

(22) Filed: Nov. 29, 2019

Related U.S. Application Data
(63) Continuation of application No. PCT/US18/63242, filed on Nov. 30, 2018.
(60) Provisional application No. 62/773,322, filed on Nov. 30, 2018.

(51) Int. Cl. A61K 47/68; A61K 31/704; A61K 31/337; A61K 33/243; A61K 51/10; C07K 16/22; C07K 16/32; C07K 16/18; A61K 47/69; A61P 35/00; A61P 35/04

(52) U.S. Cl. CPC A61K 47/6845; A61K 47/6855; A61K 31/704; A61K 31/337; A61K 47/6843; A61K 33/243; C07K 2317/24; C07K 16/22; C07K 16/32; C07K 16/18; A61K 47/6929; A61P 35/00; A61P 35/04; A61K 51/1096

(57) ABSTRACT

Methods and compositions for enhancing the therapeutic activity of agents at low doses as a result of improved targeted drug delivery, namely precision delivery, are described. The methods and compositions can be used to decrease the effective administered amounts of active therapeutic agents via precision delivery to targets capable of mediating active transport across the biological barrier formed by the vasculature that lines blood vessels. Antibodies (and other targeting agent species) to precisely deliver an active ingredient (i.e., a therapeutic agent) to cells expressing, for example, a target protein on their cell membranes, are also described, as are methods of treating disease.

### RELATED APPLICATIONS

This application claims the benefit of and priority to U.S. provisional patent application Ser. No. 62/773,322, filed 30 Nov. 2018 (attorney docket no. PRI-0100-PV3), and International patent application serial no. PCT/US2018/063242, filed 30 Nov. 2018 (attorney docket no. PRI-0100-PC2), each of which has the same title as and is commonly owned with the instant application, and each of which is hereby incorporated by reference in its entirety for any and all purposes.

### GOVERNMENT SUPPORT

The inventions are supported, in whole or in part, by grants U01 CA193787, R01 CA169644, and P01 HL119165 from the U.S. National Institutes of Health (NIH) and U.S. Department of Defense award W81XWH-11-1-0693. The Government has certain rights in the invention.

### INTRODUCTION TO THE INVENTION

Poor delivery efficiency continues to hamper the effectiveness of targeted medicines engineered to selectively seek and destroy diseased cells, such as cancerous cells that form solid tumors. (Bae and Park, 2011; Bommareddy et al.; Chrastina et al., 2011; H. F. Dvorak et al., 1991; Gupta et al.; Huang et al., 1997; Lazarovits et al., 2015; D. P. McIntosh et al., 2002; Oh et al., 2007; Oh et al., 2004; Oh et al., 2014; Park, 2013; Schnitzer, 1998; Sousa et al., 2016; Wang et al., 2012; Weathers and Gilbert, 2016; Wilhelm et al., 2016; Xu et al., 2017; Zhu and Pauli) In this era of precision medicine with its rapidly expanding arsenal of nanodrugs and immunotargeted therapies, tremendous progress has been made in the treatment of hematological malignancies that, by nature, are not protected by vascular barriers (Bae and Park, 2011; Lazarovits et al., 2015; Park, 2013; Ruoslahti et al., 2010; Sousa et al., 2016; Sykes et al., 2014; Wang et al., 2012; Weathers and Gilbert, 2016; Wilhelm et al., 2016; Xu et al., 2017). In diseases affecting solid tissues, (such as solid tumors and metastatic lesions), however, modest or absent pharmacological activity exhibited by the most promising therapies in clinical trials could be improved significantly with better delivery systems that bypass vascular barriers. Targeting strategies have evolved to avoid vascular barriers and a dependence on passive delivery by shifting their focus from cells and targets inside diseased cells to endothelial cells (EC) exposed directly to blood (Hajitou et al., 2006; Lazarovits et al., 2015; Ruoslahti et al., 2010; Schnitzer, 1998; Sykes et al., 2014; Wilhelm et al., 2016). Accessible targets on luminal EC surfaces in vivo have enabled tissue-specific delivery to vessel surfaces. However, the long-standing delivery challenge remains of getting beyond EC surfaces and barriers to concentrate drugs inside target tissue.

Our overall goal is to boost precision drug delivery into solid disease tissue, including tumors and metastatic lesions, as well as organs such as the lung, by exploiting a highly precise and active transport pathway that we discovered (Carver and Schnitzer, 2002; Carver and Schnitzer, 2003; Carver and Schnitzer, 2007a, 2007b; Carver et al., 2003; Chrastina et al., 2011; Chrastina and Schnitzer, 2010; Durr et al., 2004; Griffin and Schnitzer, 2008; Massey and Schnitzer, 2009; McIntosh et al., 2000; D. P. McIntosh et al., 2002; D. P. McIntosh et al., 2002; Oh et al., 2007; Oh et al., 2004; Oh et al., 2014; Schnitzer, 1997a; Schnitzer, 1998, 2001; J. E. SchnitzerJ. Liu et al., 1995; Schnitzer and Oh, 1995; Schnitzer et al., 1994; J. E. Schnitzer P. Oh et al., 1995) and that could result in vastly improved therapy and reduced toxicity for patients suffering from a wide variety of diseases. Here, we define precision delivery to be the rapid, highly precise and active delivery of systemically administered agents directly into the desired target tissue. Our approach is intended to go well beyond so-called ‘active targeting’ that ultimately relies on passive transvascular exchange to get across the EC barrier and enter inside the tumor where the agent can “actively” interact or bind to its specific target, usually on the surface of the tumor cell. All current molecular therapies (including small drug chemotherapeutic, antibodies, and nanoparticles) rely on passive delivery routes across the endothelium (BrownHarrist et al., 1995; BrownYeo et al., 1995; Dvorak, 2002; Dvorak et al., 1995; Dvorak et al., 1988; Dvorak et al., 1999; H. F. Dvorak et al., 1991). This passive transendothelial delivery tends to be slow and inefficient, largely because it depends on the concentration gradient of the drug across the vascular EC barrier: the larger the dose, the higher the drug concentration in the blood that is used to generate the driving force needed for faster and greater dose delivery into target diseased tissue.

Although the vascular endothelium does form a formidably restrictive blood:tissue interface (Burrows and Thorpe, 1993; Carver and Schnitzer, 2003; H. F. Dvorak et al., 1991; D. P. McIntosh et al., 2002; Schnitzer, 1998), the molecular variations present on the surface of vascular ECs in vivo does represent opportunities to target discrete vascular beds, to traverse blood vessels and to gain access to the underlying tissue. The vascular endothelium, and EC surface specifically, is inherently, directly and rather immediately accessible to agents injected into, and circulating, in the blood. To exploit this potential, we developed a novel “proteomic-imaging” approach that integrates state-of-the-art global analytical and imaging techniques to rapidly identify and validate proteins expressed at the luminal EC surface in vivo. We shifted away from the standard analytical focus of current “-omic” efforts on disease biomarker targets, which unfortunately exist deep in the tissue, to inherently accessible EC proteins that can facilitate tissue-specific delivery. We discovered and validated unique proteins on vascular ECs from different organs and tumor tissue (Durr et al., 2004; Li et al., 2009; D. P. McIntosh et al., 2002; Oh et al., 2007; Oh et al., 2004; Oh et al., 2014; TestaChrastinaLi et al., 2009; TestaChrastinaOh et al., 2009; Valadon et al., 2006). More importantly, we have performed proteomic-imaging analysis of specialized vesicular transporters at the luminal EC surface called caveolae and have demonstrated that they are accessible to antibodies injected intravenously and can selectively pump targeted antibody with attached cargo across the endothelium and into underlying tissue in vivo (Dun et al., 2004; D. P. McIntosh et al., 2002; Oh et al., 2007; Oh et al., 2004; Oh et al., 2014; Valadon et al., 2006).

These discoveries enable new precision delivery that approaches ideal targeting and is well beyond current so called targeted delivery. Our most recent studies have identified a cleaved form of Annexin A1 (AnnA1) as a key delivery target that is selectively concentrated in caveolae of tumor endothelium and that is sufficiently specific and accessible to circulating molecular probes (i.e. antibodies) to enable tumor targeting and active penetration in vivo (Oh et al., 2014). Monoclonal antibodies against AnnA1 (mAnnA1) that recognizes human, rat and mouse AnnA1, were shown to enter and bind tumor endothelial caveolae and be rapidly, precisely, and actively pumped across the tumor EC barrier, and even concentrate most of the dose only inside rodent mammary tumors within 1-2 hr of intravenous (i.v.) injection (Oh et al., 2014). Previous work also identified aminopeptidase 2 (APP2) that is concentrated in caveolae of lung EC in vivo (Oh et al., 2007); dynamic imaging studies revealed the ability of caveolae to pump targeted monoclonal antibodies against APP2 (mAPP2) rapidly and precisely across the endothelium in the lung within seconds to minutes of i.v. injection (Oh et al., 2007).

Our innovative EC surface mapping strategy and caveolae targeting strategy creates new opportunities to develop tissue-specific delivery platforms for therapy. By targeting a transvacular pumping system, we have opened a specific gateway across the blood:tissue interface and have advanced drug delivery much closer to achieving the ideal of precision medicine. The extraordinary ability of the caveolae pumping system to transport targeted agents not passively with, but actually actively against, a large concentration gradient and deliver them, even at very low doses, precisely inside a specific target tissue; this theoretically offers the means to concentrate pharmaceutical agents inside a diseased tissue with unprecedented tissue-specificity, thereby resulting in less toxic exposure in normal tissues and greater efficacy at significantly reduce (i.e. ultra low) doses. We have identified the first class of antibodies to be pumped across the EC barrier and thus to penetrate actively a single tissue (organ or solid tumors).

For decades, a solution has been sought to the huge delivery problem plaguing modern targeted medicine. It has been readily obvious for a century or more that: i) nearly all drugs can be toxic and frequently begin to be therapeutically effective only at high, sometimes near toxic doses; and ii) getting drugs only to the desired site of action is necessary to avoid unwanted side effects and to maximize drug efficacy. The concept of ideal delivery has been clear but unattainable. Ideal targeting gets all of the imaging or therapeutic agent precisely, rapidly, and comprehensively to the desired location—usually the desired tissue or cell. In so doing, it concentrates the drug at the site of desired pharmacological activity and eliminates exposure to other cells and tissues where unwanted, off target effects can be realized. If possible, it has been clear for decades that many potent and potentially effective drugs that could benefit patients many times are eliminated in clinical testing by their unwanted toxicities, obviously resulting from exposure to off target cells and tissues. For diseases located in solid tissues and not primarily in the circulating blood, all current i.v. therapies, whether labeled targeted or not, do not come close to achieving ideal targeting and are limited by toxicities produced at the rather high dosages required for therapeutic efficacy to be possible and revealed. Targeted delivery is usually miniscule, at levels well below 1% of injected therapeutic dose and frequently even less than 0.001%. To designate this delivery as targeted seems to be a stretch far below theory.

Yet the age of targeted molecular medicine has been ongoing for decades based more on intent and design than actual delivery and reality. We have indeed created drugs and biologics with great specificity and potency; each can stimulate or inhibit a single target to yield a single effect, whether it be a signaling pathway or a functional biomarker specific to a cell population. We have even created antibody-drug conjugates to enhance the target delivery of very potent and usually extremely toxic agents for cancer therapy; success in hematogenous cancer seems more forthcoming, but antibody delivery into solid tumors is well less than 1% of the injected mg/kg doses. The current approach is to increase the toxic load per antibody (use more toxic agents and/or attach more drug to each antibody). Stable linkages between toxin and antibody are required to minimize toxin release in the blood and rapid systemic toxicity. But the fundamental delivery problem across the tumor EC barrier remains.

In the case of cancer patients, many will receive at least one form of radiation for imaging or therapy. Radiation kills tumor cells quite effectively. Yet, many treated patients have unwanted, dose-limiting side effects and too-short remissions. External beam radiation can be effective when fractionated optimally but it can also expose nearby, sometimes critical, normal tissues to radiation. Targeted radionuclide therapy has over 50 years become most effective for hematogenous tumors because the targeting moiety, usually an antibody, has immediate intravenous access to its target, thereby readily delivering the desired therapeutic radionuclide specifically to the tumor cells. Solid tumors lack similar target accessibility and responsiveness, primarily because vascular EC and other barriers limit passive tissue entry of the radioconjugate from blood. Dose escalations are necessary yet capped by severe toxicities. Moreover, the limitations of current radioimmunotherapy (RIT) approaches extend to their applications as imaging agents that also rely on the passive transvascular delivery and retention to create a strong tumor-specific signal. Frequently, it can take up to several days to clear imaging probe from the blood and other tissues before tumor signals can be separated from the background noise.

Tumor interstitial radiation brachytherapy has evolved as an effective alternative to systemic therapies by directly injecting each tumor with multiple radioactive seeds arranged geometrically to optimize coverage. For example, mechanical implantation of ¹²⁵I-loaded seeds into the tumor interstitium is widely applied clinically because of its curative effect, minimal surgical trauma, and few complications. ¹²⁵I trapped inside each seed provides a low intensity yet sustained exposure better confined to its immediate space than other radionuclides. Despite not being considered potent enough for radioimmunotherapy, ¹²⁵I can be quite toxic to individual tumor cells and destroys adjacent tissue within 4 mm of the seeds in a graded fashion. For those tumors that are surgically accessible and amenable to repeated injections, such mechanical impregnation, although invasive and laborious, provides inherent precision in radiation delivery, bypasses exposure of blood and other organs, and avoids the typical, dose-limiting toxicities of systemic radiotherapies.

Here, we have not only overcome the barrier problem but proven for the first time that doing so enables unprecedented therapeutic efficacy for multiple therapeutic modalities in multiple diseases. It also enables imaging of solid tumors at a speed and selectivity not observed previously. This new approach is indeed leading to a paradigm shift in imaging and molecular medicine and provides a major step towards ideal targeting well beyond all current strategies deemed to be “targeted” today.

The ever-growing pharmacopeia of specific and potent therapeutic agents has encouraged the bold proposal of personalized medicine and recently precision medicine, basically attempting to tailor medicinal therapy to the drivers of disease as revealed through the “omic” screening of each patient. We have achieved “precision” diagnostic evaluation through genomics technology and “precision” drugs in the sense of target/pathway specificity and potency. But, without good access to the desired target, we may once again achieve less than the desired clinical responses. Until precision in delivery can also be attained, the full benefits of medicine's great technological achievements may appear more incremental to the public than substantial. Here we describe the first precision delivery, imaging, and therapy system all integrated through the caveolae pumping system to show for the first time unprecedented rapid targeting of imaging and therapeutic agents to yield efficacy at ultra-low doses that still yield effects more than 10- to 100-fold or more of the maximum tolerated dose of an untargeted (i.e., non-targeted) form of the same therapeutic agent.

### Caveolae Pumping System

Existing drug interventions rely on passive transvascular delivery of the drug that depends on a large concentration gradient across the semi-permeable vascular endothelial barrier. The passive forces driving the circulating drug from the bloodstream into solid diseased tissue results in poor delivery and insufficient access to cells. Only a very small amount of the drug (<<1%) actually reaches and penetrates target tissue to achieve what is usually suboptimal therapeutic effect (H. F. Dvorak et al., 1991; Huang et al., 1997; Wilhelm et al., 2016). Hence, ever increasing doses must be administered to the point of generating harmful side effects elsewhere in the body, yet still resulting in poor response, high relapse rates, and the development of drug resistance. Accessible targets on luminal EC surfaces in vivo have enabled tissue-specific delivery to vessel surfaces as a means to improve targeted drug delivery. However, the long-standing challenge remains to get beyond the EC surface and barrier actually to concentrate targeted agents inside tumors.

Here we focus on overcoming the barrier formed by the vascular endothelium. The overall goal is to boost drug delivery into diseased tissue by exploiting a highly precise active transport pathway that could result in improved precision drug delivery and therapy while possibly reducing toxicities for patients.

Caveolae pumping system: Our search for ways to go beyond passive transvascular delivery led to a breakthrough discovery of a transvascular pumping system in vascular endothelium in lung first and then in solid tumors (Oh et al., 2007; Oh et al., 2014). Caveolae at the EC surface can precisely, rapidly, and actively transcytose a selected targeted moiety, such as specific antibodies, out of the bloodstream and into the underlying tissue (Chrastina et al., 2010; D. P. McIntosh et al., 2002; Oh et al., 2007; Oh et al., 2004; Oh et al., 2014; Schnitzer, 1998, 2001). Caveolae are ˜60-80 nm omega-shaped plasmalemmal invaginations that are distinct from clathrin-coated vesicles and act as dynamic transport vesicles (Oh et al., 1997; Schnitzer, 2001; J. E. Schnitzer J. Liu et al., 1995; J. E. Schnitzer et al., 1995; Schnitzer et al., 1994; Schnitzer et al., 1996) mediating endocytosis in many cell types and transcytosis, particularly in endothelium (Carver and Schnitzer, 2002; Schnitzer, 1997a, 1997b; Schnitzer, 1998, 2001; Schnitzer and Oh, 1994; Schnitzer et al., 1994). We used proteomic-imaging technology to discover that EC caveolae can express tissue- and disease-specific proteins, including tumor EC markers, and that targeting tumor- or organ-specific proteins enriched in EC caveolae provide a novel way to overcome the restrictive EC barrier; caveolae-targeting antibodies are pumped across the EC barrier and concentrate inside target tissue (Carver and Schnitzer, 2003; Carver and Schnitzer, 2007b; Chrastina et al., 2010; Dun et al., 2004; D. P. McIntosh et al., 2002; Oh et al., 2007; Oh et al., 2004; Oh et al., 2014; Schnitzer, 1998, 2001; Valadon et al., 2006). We have identified a novel transvascular transport pathway capable of pumping antibodies directly into target tissue (Oh et al., 2007; Oh et al., 2014); in the case of tumors, most of the injected dose rapidly penetrates the solid tumors within 1-2 hr (Oh et al., 2014).

IVM imaging system and new ectopic-orthotopic mammary tumor models. As part of the preclinical testing of our candidate caveolae-targeting antibody, we needed to define how it is processed by normal and tumor EC in vivo. We developed a new IVM tumor model imaging system to observe directly and continuously the binding and delivery of mAnnA1 armed with therapeutic cargo across the EC barrier. This system includes computational algorithms developed by us to assess and quantify EC surface binding and transvascular fluxes from digital movies (Oh et al., 2007). We first detailed our dynamic IVM imaging system in lung tissue to visualize CTA binding and EC processing in live nude mice (Oh et al., 2007). We proved that caveolae operate effectively as transvascular pumps, moving the lung-specific CTAs within 60 seconds from blood across the EC barrier and deep into the lung tissue, even against a concentration gradient (Oh et al., 2007).

Next, we developed novel mammary tumor models amenable to IVM to allow us to open the “black box” of the solid tumor and see inside. IVM offers an unparalleled view into tumor development with dynamic, high resolution and continuous in vivo imaging of molecular and cellular events. Many tumor models have been used with IVM, including the cranial window model and by directly exposing an internal organ for short term imaging (Jain, 2002; Laudanna and Constantin, 2003). However, performing continuous long term IVM in these models is technically challenging. The dorsal skinfold window chamber system offers greater ease of use and permits live dynamic imaging of the implanted tumor for several weeks. Subcutaneously implanted tumors are the most popular and current IVM standard, but for studying many solid tumors, they lack proper orthotopic stroma and tumor microenvironment. They may not duplicate human disease and appear to respond therapeutically to many single therapies not found to be as effective in clinical trials and cancer patients.

As published (Borgstrom et al., 2013), we expanded the preclinical utility of the IVM tumor imaging system by creating an orthotopic tissue environment for the tumor in the ectopic dorsal skin (i.e. ectopic-orthotopic or EO model). We co-implanted tumor cell spheroids with healthy orthotopic donor tissue into dorsal skin window chambers. The co-implantation of healthy orthotopic tissue substantially impacts the tumor microenvironment and tumor growth in several ways. EO tumors, unlike subcutaneous tumors, exhibit resistance to an array of standard cancer monotherapies (e.g. doxorubicin, cisplatin, paclitaxel), as well as single immune-therapies (e.g. Herceptin, anti-VEGF). EO tumors advance beyond subcutaneous tumors used in IVM by providing orthotopic tumor microenvironment and by lacking excessive therapeutic sensitivity that does not reflect most solid tumor patients who do not respond to these monotherapies, require combination therapies, or acquire resistance. These drug-resistant models may better represent the hard-to-treat patients.

Fluorescence IVM of EO tumors following IV injection of fluorophore-labeled mAnnA1 readily revealed even at low magnifications that mAnnA1 precisely and rapidly accumulated throughout the tumor (Oh et al., 2014). Despite the very low IV dose (3 μg fluoro-conjugate mAnnA1) and the resultant low to nil signal detected inside the blood vessels at all times, the signal inside the tumor tissue continued to increase within minutes of injection until approaching image saturation at 60 min. The fluorescence signals from the “red” tumor cells and the “green” mAnnA1 clearly overlap; the antibody floods the tumor throughout, but not the surrounding tissue. Examination at higher magnification showed rapid antibody binding to the luminal EC surface with subsequent pumping across the endothelium and into the perivascular space occurring robustly within 20-60 min of IV injection (Oh et al., 2014). Unlike passive transport that must follow the concentration gradient, IVM showed that antibody transport across endothelium and into tumors occurred even against a considerable concentration gradient, which constitutes, but definition, active transport or pumping. Despite the opposite diffusive force from low blood to high tissue gradient, the antibody continues to be transported into the tumor. The antibody actively penetrates the tumor and is rapidly concentrated inside the tumor at levels well beyond the highest blood concentration. Further quantification and analysis as reported in detail (Oh et al., 2014) all support an active transport mechanism, consistent with tumor EC caveolae functioning as transvascular pumps. IVM also showed that this transvascular pumping of mAnnA1 occurs in breast, prostate and lung tumors with equivalent kinetics and degree of uptake. Thus, the slower kinetics of the caveolae pumping system in tumors versus lungs is noteworthy and discussed (Oh et al., 2014). Together, these results demonstrate unprecedented imaging, antibody targeting and tumor penetration and clearly show that mAnnA1 is pumped across the EC barrier to actively penetrate solid tumors.

### Caveolae Targeting Enhances Drug Potency

Caveolae can pump and concentrate targeted antibodies with attached cargo inside tumors and solid tissues, even at low doses and against a concentration gradient. Tumor uptake far exceeds other antibodies and passive transvascular delivery. The ability to pump select therapeutic and imaging agents across EC barriers may enable a shift away from the current, passive transvascular delivery paradigm towards using an active portal to deliver agents directly inside diseased tissue.

Our publications (Oh et al., 2007; Oh et al., 2014) were the first to describe the caveolae pumping system and a new class of delivery target and targeting agent, namely, the caveolae-targeting agent (CTA), we generated (i.e. mAnnA1 and mAPP2) which were the first probes to actively penetrate a single tissue (solid tumors and lung) after injection. We have now generated a new class of therapeutic agents based on the caveolae pumping system and CTA to which we linked a broad range of commonly used drug therapies. In so doing, we have created the first precision delivery agents useful for imaging and therapy that in direct head-to-head comparisons in disease models, shows vastly enhanced efficacy well beyond current targeted and non-targeted therapies. This new distinctive approach to overcome the vascular barrier to enable rapid tissue penetration by providing a direct route into underlying diseased tissue. As the examples below clearly demonstrate, specific transendothelial pumping to concentrate active agents with a high degree of precision inside diseased tissue can significantly boost the therapeutic potency of active agents at ultra low doses (in some cases, orders of magnitudes lower of than the maximum tolerated dose) when compared to the standard effective administered amount. This discovery, especially the magnitude of therapeutic improvement, was unexpected, surprising, even contrary to the many scientists that predicted little benefit from this unusual approach.

To further illustrate the unexpected benefits of using a precision delivery system based on the caveolae pumping system and CTA to increase therapeutic potency at ultra low doses, it is important to consider the current delivery paradigm. It relies predominantly on passive transvascular delivery to move drugs out of the blood into diseased tissue. All current drugs and imaging agents that must cross the vascular wall to reach the diseased cells do so passively, regardless of whether they are considered a targeted agent or not. By way of non-limiting examples, a targeted therapeutic agent would include a specific antibody (e.g. Herceptin), specific small drug (e.g. tyrosine kinase inhibitor), or antibody-drug conjugate are examples of targeted therapeutic agents, while typical chemotherapeutic agents (e.g., cisplatin, paclitaxel, unconjugated radiopharmaceutical agents, etc.) or nanocarriers (e.g., Doxil) are examples of non-targeted agents. Interacting with a specific therapeutic target does not obviate this passive delivery. As noted earlier, dependence on passive transvascular delivery necessitates the use of high doses to generate a large concentration gradient across the EC barrier in order to drive more drug from the bloodstream to the inside (interstitium and parenchyma) of the diseased tissue, such as tumors. In this case, typical doses of 1-30 mg/kg are required for any therapeutic response to occur, thereby administering ˜100 mg to even more than ˜1 g of drug in an attempt to treat tumors that frequently are well less than 100 ml in volume—a dose (if properly and precisely delivered) readily produces >10 mM levels of small drugs and ˜100 μM of antibodies inside the tumor. Most of the drugs are fully active when directly applied to cells at low μM to high nM concentration for standard chemotherapeutics and at low to even sub-nM levels with modern targeted agents. Despite this dosage overkill and achieving blood levels above 1 μM (and even beyond 100 μM), drugs designed and confirmed to be effective at nanomolar concentrations or less, have difficulty reaching intra-tumoral therapeutic concentrations that enable their full potency.

We have discovered that transvascular pumping of targeted agents into tumors and other diseased tissue can overcome this problem and enhance therapeutic potency or therapeutic index at ultra low doses (e.g., μg/kg to ng/kg dose ranges). This can be done in a wide variety of contexts, including tumors that are very difficult to treat, such as multi-drug resistant (MDR) tumors, large tumor burden, and metastatic tumors. By way of non-limiting example, we have created a new interstitial radiation modality using an innovative radioimmunotherapy approach and delivery mechanism that impregnates and traps radioisotopes such as ¹²⁵I inside the tumor interstitium quickly, precisely, and quite homogeneously. We were able to reduce the size of the ¹²⁵I-seed from millimeter (mm) scale to nanometer (nm) scale and achieve precision tissue impregnation not invasively by multiple direct injections but rather intravenously by coupling ¹²⁵I to CTA that is pumped across the endothelium to rapidly and actively penetrate solid tumors. The concentrating effect inside tumor provided by caveolae pumping makes it possible to deliver sufficient quantities of pharmaceutical agents to image and treat both resistant and metastatic solid tumors.

In oncology, using caveolar transport to enable precision delivery to tumors could profoundly alter treatment outcomes for many forms of cancer and enable powerful new approaches, including in vivo functional imaging, nanoparticle-based drugs and precision oncology regimens that specifically tailor treatment strategies to patients based on genomic profiling. Additionally, promising therapies that had been abandoned due to toxicity could be re-engineered to target tumors in a manner that bypasses normal tissue. This overall approach has broad applicability to other diseases types affecting solid tissues and organs. Here we achieve through precision delivery the ability to enhance the therapeutic index of pharmaceutical active agents at ultra low doses and expand the treatment window available to oncologists by orders of magnitude.

With regard to solid cancers, it is not currently known the extent to which changes in the tumor microenvironment that impact vascular barriers contribute to the development of drug resistance in metastatic cancers. Recent studies (Frentzas et al., 2016) suggest co-opted vessels with mature vascular barriers account for nearly half of the vessels in liver metastatic lesions. By enhancing local intratumoral concentrations of therapeutic agents, a caveolar-based precision delivery system could remove the uncertainty about whether effective therapeutic doses are reaching tumor sites and would enable instead, a focus on elucidating other mechanisms that may be at work. Subsequent drug failures would become more meaningful when treatments become more powerful at low dosages. Transvascular pumping of therapeutic compounds into solid tumors and diseased tissue will maximize therapeutic impact for many agents treating cancer and other diseases.

### Definitions

Before describing the instant invention in detail, several terms used in the context of the present invention will be defined. In addition to these terms, others are defined elsewhere in the specification, as necessary. Unless otherwise expressly defined herein, terms of art used in this specification will have their art-recognized meanings.

“Affinity” refers to the strength of the sum total of noncovalent interactions between a single binding site of a molecule (e.g., an antibody) and its binding partner (e.g., an antigen). Unless indicated otherwise, “binding affinity” refers to intrinsic binding affinity, which reflects a 1:1 interaction between members of a binding pair (e.g., antibody and antigen). The affinity of a molecule X for its partner Y can generally be represented by a dissociation constant (Kd). Affinity can be measured by common methods known in the art, including those described herein. Specific illustrative and exemplary embodiments for measuring binding affinity are described elsewhere herein. Binding affinity of an antibody (or other targeting agent) can be measured using any suitable technique, e.g., by a radioimmunoassay (RIA) or by Scatchard analysis or by surface plasmon resonance (e.g., via a Biacore). In certain embodiments, a targeting agent has a dissociation constant (Kd) of 0.1 uM, 100 nM, 10 nM, 1 nM, 0.1 nM, 0.01 nM, or 0.001 nM (e.g., 10⁷M or less, e.g., from 10⁷M to 10¹³M.

An “affinity matured” antibody refers to an antibody with one or more alterations in one or more hypervariable regions (HVRs), compared to a parent antibody that does not possess such alterations. Preferably, such alterations result in improved affinity of the antibody for its target antigen.

The term “antibody” herein is used in the broadest sense and encompasses various antibody structures, including, but not limited to, monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

The terms “full length antibody,” “intact antibody,” and “whole antibody” are used herein interchangeably to refer to an antibody having a structure substantially similar to a native antibody structure or having heavy chains that contain an Fc region as defined herein.

The term “Fc region” herein is used to define a C-terminal region of an immunoglobulin heavy chain that contains at least a portion of the constant region. The term includes native sequence Fc regions and variant Fc regions.

The term “chimeric” antibody refers to an antibody in which a portion of the heavy and/or light chain is derived from a particular source or species, while the remainder of the heavy and/or light chain is derived from a different source or species.

A “human antibody” is one that possesses an amino acid sequence corresponding to that of an antibody produced by a human or a human cell or derived from a non-human source that utilizes human antibody repertoires or other human antibody-encoding sequences. This definition of a human antibody specifically excludes a “humanized” antibody comprising non-human antigen-binding residues.

A “humanized” antibody refers to a chimeric antibody comprising amino acid residues from non-human hypervariable regions (HVRs) and amino acid residues from human framework regions (FRs). In certain embodiments, a humanized antibody will comprise substantially all of at least one, and typically two, variable domains, in which all or substantially all of the HVRs (e.g., complementarity determining regions or “CDRs”) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

An “effective amount” of an agent, e.g., a targeted drug conjugate, an therapeutic agent, a pharmaceutical formulation, etc. refers to an amount effective, at dosages and for periods of time necessary, to achieve the desired therapeutic or prophylactic result.

An “individual” or “patient” or “subject” is a mammal Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “isolated” molecule is one that has been separated from a component of its natural environment. In some embodiments, for example, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC).

The term “monoclonal antibody” refers to an antibody obtained from a population of substantially homogeneous antibodies, i.e., the individual antibodies comprising the population are identical (as assessed at the level of Ig heavy and/or light chain amino acid sequence) and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, but not limited to, the hybridoma method, recombinant DNA methods, phage-display methods, and methods utilizing transgenic animals containing all or part of the human immunoglobulin loci, such methods and other exemplary methods for making monoclonal antibodies being described herein.

The term “package insert” is used to refer to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

The term “pharmaceutical composition” refers to a preparation that is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

A “patentable” composition, machine, method, process, or article of manufacture according to the invention means that the subject matter satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically exclude the unpatentable embodiment(s). Also, the claims appended hereto are to be interpreted both to provide the broadest reasonable scope, as well as to preserve their validity. Furthermore, if one or more of the statutory requirements for patentability are amended or if the standards change for assessing whether a particular statutory requirement for patentability is satisfied from the time this application is filed or issues as a patent to a time the validity of one or more of the appended claims is questioned, the claims are to be interpreted in a way that (1) preserves their validity and (2) provides the broadest reasonable interpretation under the circumstances.

The term “species”, when used in the context of describing a particular compound or molecule species, refers to a population of chemically indistinct molecules.

A protein that is “associated with caveolae”, “caveolae-associated”, or the like refers to a cell-surface protein located in proximity or otherwise effecting caveolae function, formation, stability, or activity. Examples of caveolae-associated proteins expressed in normal or diseased tissue include aminopeptidase P2 (APP2), annexin A1 (AnnA1) including a truncated 34 kD form of the protein, caveolin-1 (CAV1), caveolin-2 (CAV2), caveolin-3 (CAV3), Cavin-1 (also known as polymerase-1 and transcript release factor) (PTRF), Cavin-2, Cavin-3, EDH2, GlycosylPhosphatidyllnositol (GPI)-linked receptors, Pacsin2 (also referred to as syndapin2), Flotillin-1, Flotillin-2 and Cavin-4. Additional proteins “associated with caveolae”, “caveolae-associated”, or the like also refer to proteins isolated from luminal vascular endothelial cell membranes enriched for caveolae and include APP2, CD34, OX-45, AnnA1, vascular endothelial growth factor (VEGF) receptors-1 and -2, Tie2, aminopeptidase-N, endoglin, carcino-embryonic antigen-related cell adhesion molecule 1 (CD66), (C-CAM-1) and neuropilin-1, annexin A8, ephrin A5, ephrin A7, myeloperoxidase, nucleolin, transferrin receptor and vitamin D-binding protein.

The term “therapeutic index” (or “TI” or “therapeutic ratio”) refers to the dose ratio between a drug's toxic and therapeutic (or prophylactic) effects. TI generally refers to the dose of a drug (or lead compound, drug candidate, or the like) that causes adverse effects at an incidence or of a severity that is incompatible with the desired effect(s) (e.g., the toxic dose in 50% of cells, study animals, or other subjects or patients (TD₅₀)) compared to the minimum effective dose (the minimum effective dose in an assay, in 50% of a patients or a population of study subjects (human or non-human) (ED₅₀). As will be appreciated, a higher TI is preferable to a lower TI, as a higher TI means that a larger amount of the compound would be required to observe toxic effects as compared to the amount needed to efficacious. The invention provides for increasing a particular therapeutic agent's TI, preferably by at least a factor of about 10, even more preferably by a factor of 20-10,000 or more. For example, if drug A when untargeted has a TI of X, in the context of the invention the targeted form of drug A will have a TI of at least about 10×, preferably 100× to 10,000× or more. The related term “therapeutic window” refers to the range of dosages between efficacy and toxicity. As is known, drugs with small therapeutic indices or therapeutic windows must be administered with care and control in order to avoid toxicity.

The term “therapeutic potency” refers to the amount of a compound (e.g., an drug, a lead compound, etc.) required to produce an effect of a given intensity. Highly potent compounds produce a particular effect at low concentrations. As with therapeutic indices, the invention provides for increasing a particular therapeutic agent's therapeutic potency, preferably by at least a factor of about 10, even more preferably by a factor of 20-10,000 or more. For example, if drug A when untargeted has a potency of X, in the context of the invention the targeted form of drug A will have a potency of at least about 10×, preferably 100× to 10,000× or more.

By “consisting essentially of” is meant that additional component(s), composition(s), or method step(s) that do not materially change the basic and novel characteristics of the present invention may be included in the compositions or kits or methods of the present invention. Such characteristics include the ability to selectively detect target nucleic acids in biological samples (e.g., whole blood or plasma). Any component(s), composition(s), or method step(s) that have a material effect on the basic and novel characteristics of the present invention would fall outside of this term.

Where a range of values is provided in this specification, it is understood that each intervening value, to the tenth of the unit of the lower limit unless the context clearly indicates otherwise, between the upper and lower limit of that range, and any other stated or unstated intervening value in, or smaller range of values within, that stated range is encompassed within the invention. The upper and lower limits of any such smaller range (within a more broadly recited range) may independently be included in the smaller ranges, or as particular values themselves, and are also encompassed within the invention, subject to any specifically excluded limit in the stated range. Where the stated range includes one or both of the limits, ranges excluding either or both of those included limits are also included in the invention.

As used herein, the singular forms “a”, “an”, and “the” include plural references unless the context clearly dictates otherwise.

The term “about” refers to approximately a +/−10% variation from the stated value. It is to be understood that such a variation is always included in any given value provided herein, whether or not it is specifically referred to.

### SUMMARY OF THE INVENTION

The object of this invention is to provide patentable targeted drug conjugates, compositions containing such conjugates, kits containing such conjugates and compositions, and methods for making and using the same.

Thus, in one aspect, the invention concerns targeted drug conjugates that comprise an active ingredient (i.e., a therapeutic agent) and a targeting agent. In some embodiments, the targeted drug conjugate comprises a therapeutic agent conjugated directly or indirectly (i.e., via an intermediate chemical moiety such as a linker, dendrimers, etc.) to a targeting agent, whereas in other embodiments, the therapeutic agent and targeting agent of a targeted drug conjugate are merely associated in a composition, e.g., as part of a nanoparticle (e.g., a liposome encapsulating the therapeutic agent and having the targeting agent displayed on the liposome's outer surface). In general, the active ingredient is a therapeutic agent. When part of a targeted drug conjugate of the invention, the potency of the therapeutic agent is enhanced as compared to untargeted forms of the same therapeutic agent such that when formulated into a suitable composition, for example, a pharmaceutical composition comprising the targeted drug conjugate and a pharmaceutically acceptable carrier, at least about 10-fold less (e.g., 10-10,000-fold less, e.g., 20-, 50-, 100-, 500-, 1,000-fold less) of the therapeutic agent is required to exert the desired effect (e.g., a prophylactic or therapeutic effect) than when an effective amount of the therapeutic agent present in an untargeted form is administered to a subject having a disease or condition amenable to treatment thereby. Any suitable assessment can be used to determine whether the amount of the therapeutic agent administered as part of a targeted drug conjugate of the invention is at least about 10-fold less as compared to an untargeted form (i.e., a composition or formulation that does include a targeting agent) of the same therapeutic agent in order to achieve substantially the same therapeutic benefit, including vitro assays, non-human animal models, and treatment of subjects.

In some embodiments, the therapeutic agent is a small molecule, a peptide, a protein, a nucleic acid, a radionuclide, or a gene delivery vehicle (e.g., a virus, preferably an engineered virus).

Useful therapeutic agents include those that are selected from among chemotherapeutic agents, immune stimulatory agents, anti-neoplastic agents, pro-coagulants, toxins, antibiotics, hormone, enzymes, and lytic agents.

In a targeted drug conjugates of the invention, the targeting agent specifically binds to an extracellular domain of a protein displayed on an outer surface of a cell membrane of a cell. Typically, the targeting agent is a member of a high affinity binding pair. Antibodies and antigen-binding antibody fragments (e.g., Fab fragments) that target an extracellular domain of a protein species expressed predominantly on the extracellular surface of endothelial cells, for example, caveolae of tumor endothelium (e.g., Annexin A1 (AnnA1)) are representative examples of suitable targeting agents in the context of the invention. In other embodiments, the targeting agent is a receptor, a ligand-binding receptor fragment, a receptor ligand, a small molecule, or an aptamer.

In some particularly preferred embodiments of this aspect, the targeting agent of the targeted drug conjugate of the composition specifically binds to an extracellular domain of a protein displayed on the outer surface of a cell membrane of a vascular endothelial cell, which protein is capable of mediating active transvascular pumping of the targeted drug conjugate across the cell into underlying diseased tissue. Preferably, the extracellular domain targeted by the targeting agent is displayed on the surface of the vascular endothelial cell is predominantly located in or is translocated to caveolae.

A related aspect of the invention concerns pharmaceutical compositions, which include a targeted drug conjugate composition of the invention wherein the carrier is a pharmaceutically acceptable carrier.

A further related aspect of the invention concerns kits. Such kits typically contain a composition of the invention packaged in a suitable container. In many preferred embodiments, the kits, or packages, also include instructions for use. In the context of pharmaceutical compositions, such kit instructions are usually a package insert, which contains not only instructions for use but also information about the pharmaceutically active ingredient of the targeted drug conjugate of the composition packaged in the particular kit.

Another aspect of the invention relates to methods of decreasing the amount of a therapeutic agent needed to effect therapy. Such methods comprise administering a targeted drug conjugate composition of the invention to a subject having a disease or condition amenable to treatment by the therapeutic agent deployed therein, thereby decreasing the amount of the particular therapeutic agent needed to treat the disease or condition.

A related aspect of the invention involves methods of treating a disease or condition afflicting (or which may later afflict, either for the first time or as a result of recurrence) a subject. Such methods also typically include administering to a subject suspected of or having a disease or condition a targeted drug conjugate composition according to the invention, thereby treating the disease or condition.

Subjects that can be treated in accordance with invention include humans or other mammals, for example, bovine, canine, equine, feline, ovine, or porcine animals.

In some embodiments that concern methods of the invention, the disease or condition to be treated is a non-hematologic cancer, an infection, inflammation, fibrosis, acute injury, infarction, or a pathological malfunction that is none of the foregoing. Representative examples of non-hematologic cancers that can be treated in accordance with the invention include solid cancers, for example, sarcomas, carcinomas, lymphomas, and metastatic lesions.

The foregoing and other objects, features and advantages of the invention will be apparent from the following more particular descriptions of preferred embodiments of the invention as illustrated in the accompanying drawings and figures.

### BRIEF DESCRIPTION OF THE FIGURES

A brief summary of each of the figures and tables described in this specification are provided below. For U.S. applications: this application contains at least one figure executed in color; copies hereof this application with color drawings may be provided upon request and payment of the necessary fee.

FIG. 1 provides several photos showing the destruction of mammary tumors using a mAnnA1-docetaxel ADC. Mammary tumor spheroids (H2B-GFP) were co-implanted into window chambers with mammary fat pad and allowed to vascularize and grow for 10 days. The mice were then injected with the indicated dose and imaged by IVM for 14 days thereafter, as indicated. The dosages are expressed as total docetaxel injected (either alone or immunoconjugated as indicated) per kg body weight.

FIG. 2 is a graph that provides growth curves from experiments described in connection with the photos shown in FIG. 1, and are based on average relative tumor size on day of treatment with standard deviation (n=4 mice per dose).

FIGS. 3A-3F relate to the same sets of experiments. FIG. 3A provides several photos showing that a conjugated mAnnA1-CMD-cisplatin ADC (21 μg/kg) causes tumor ablation in a dose dependent manner in mammary tumors. Cav1KO and AnnA1KO mice with H2B-GFP expressing mammary tumors (N202) were injected IV with the indicated dosage of mAnnA1 or mIgG conjugated to cisplatin and imaged with fluorescence video-microscopy. Representative static frames were captured at the indicated days after treatment.

FIG. 3B is a graph that shows relative tumor size in animals treated in the experiments described in connection with the data shown in FIG. 3A.

FIG. 3C are photos that show representative static frames Cav1KO mice with H2B-GFP expressing mammary tumors injected i.v. with the mAnnA1 conjugated to cisplatin (21 μg/kg) and imaged with fluorescence video-microscopy that were captured at the indicated days after treatment.

FIG. 3D is a graph that shows relative tumor size in animals treated in the experiments described in connection with the data shown in FIG. 3E.

FIG. 3E are photos that show representative static frames of AnnA1KO mice with H2B-GFP expressing mammary tumors injected i.v. with the mAnnA1 conjugated to cisplatin (21 μg/kg) and imaged with fluorescence video-microscopy that were captured at the indicated days after treatment.

FIG. 3F is a graph that shows relative tumor size in animals treated in the experiments described in connection with the data shown in FIG. 3C.

FIG. 4 provides several photos showing the destruction of prostate tumors using an mAnnA1 ADC. TRAMP C2 transgenic tumors were treated with mAnnA1 conjugated to docetaxel, doxorubicin or cisplatin at the indicated doses. Tumors were imaged using IVM for 14 days thereafter, as indicated. The graph below the photos provides growth curves based on average relative tumor size on the day of treatment with standard deviation (n=4 mice per dose).

FIG. 5 graphically represents data regarding the destruction of breast metastatic lesions in rats mediated by mAnnA1-DM1 antibody drug conjugates (ADCs). In these experiments, female rats were intravenously inoculated with 13762 MAT B III mammary adenocarcinoma cells to generate ample, well-circumscribed tumors in the lungs. Body weight curves show data from animals treated on day 21 post-cell inoculation with hAnnA1-MCC-DM1 or hAnnA1-CMD-Pt(II). One set of rats (n=3) received treatment of DM1 at 15 μg per animal (equivalent to 1.5 μg/kg of DM1) while another received two consecutive injections of the same dose on days 23 and 30. Rats receiving hAnnA1-CMD-Pt(II) were dosed at 100 μg/kg. All controls (untreated) died by days 8-14 post-treatment, while the ADCs prolonged rat lifespan for >60 days.

FIG. 6 graphically represents data showing body weight curves of the Pt(II) treated and control rats bearing metastatic tumors following injection of 13763 MAT B III cells as measured throughout the study. Treatment was initiated on day 21.

FIG. 7 shows Kaplan-Meier survival graphs of treated animals show average extension of survival by 15-30 days. Mice treated with hAnnA1-CMD-Pt(II) showed significantly less toxicity. Treatment with hAnnA1-CMD-Pt(II) conjugate extends survival in the HER2/neu spontaneous tumor model at ultra-low injected doses of Pt(II). Tumor-bearing mice were treated with hAnnA1-CMD-Pt(II) (7.5 μg, 1.5 μg/kg cisplatin) or cisplatin control (5 mg/kg).

FIG. 8A provides several photos showing the destruction of mammary tumors using AnnA1 radioimmunotherapy. H2B-GFP mammary tumor spheroids were co-implanted into window chambers with mammary fat pad and allowed to vascularize and grow for 10 days. Then the mice were imaged by IVM for 14 days after being injected via tail vein with 0.3, 1.0 or 3.0 μg of ¹²⁵I-mAnnA1, 3 μg of control 125I-IgG (Cont IgG), or untreated control (0.0 μg).

FIG. 8B shows several photos taken at the indicated times of tissue sections from control, wild-type, and CavKO animals treated using AnnA1 radioimmunotherapy.

FIG. 8C graphically represents data showing tumor fluorescence over time in animals implanted with H2B-GFP mammary tumors and treated with AnnA1 radioimmunotherapy used to generate the data represented in FIGS. 8A and 8B.

FIG. 9 is a plot of the body weights of 13762 tumor-bearing rats treated with ¹⁷⁷Lu-hAnnA1 radioimmunotherapy. Female Fisher rats were injected with 13762 MAT B III cells. 20 d after tumor cell inoculation, rats received ¹⁷⁷Lu-hAnnA1 (4 μCi/μg) or a control vehicle. This graph depicts body weight over time for individual rats treated with a vehicle or the indicated doses of ¹⁷⁷Lu-hAnnA1. While all controls died 12 days post-treatment initiation, a single dose of 15 μg ¹⁷⁷Lu-hAnnA1 prolonged rat lifespans by at least 190 days.

FIG. 10 is a graph of data from therapeutic efficacy studies in an animal model using ¹⁷⁷Lu-labeled AnnA1 antibody. GFP-labeled N202 tumor spheroids were co-implanted with mammary fat pad into window chambers. Tumors were imaged by IVM before and after injection via tail vein with 0, 0.3, 1 or 3 μg of ¹⁷⁷Lu-mAnnA1 (7 mCi/mg). Quantification of tumor signal over time in response to ¹⁷⁷Lu-mAnnA1 is plotted.

FIGS. 11A-11H show photos of the in vivo targeting of AnnA1 antibodies. Here, Her2/neu mice with spontaneous mammary tumors were injected intravenously with ¹²⁵I-mAnnA1 antibodies (FIGS. 11A-11D) or with isotype matched ¹²⁵I-IgG (FIGS. 11E-11H). SPECT-CT images were acquired at the indicated times. Region of interest (ROI) analysis shows the tumor-specific signal persists (encircled regions demarcate tumors). ROI was measure using the program LumaGEM_P (GammaMedica) after acquiring images using LumaGEM_A (GammaMedica). FIG. 11A is a SPECT-CT image cross-section taken at the indicated time. FIG. 11B is an ROI analysis corresponding to the SPECT-CT image in FIG. 11A. FIG. 11C shows a different cross-sectional SPECT-CT image (upper panel) taken at the indicated time with a corresponding ROI analysis (lower panel). FIG. 11D shows cross-sectional images in approximately the same plane as represented in FIG. 11C, with the upper left and upper right panels showing SPECT-CT images and the lower left and lower right panels showing corresponding ROI analyses.

FIG. 11E is a SPECT-CT image cross-section taken at the indicated time. FIG. 11F is an ROI analysis corresponding to the SPECT-CT image in FIG. 11E. FIG. 11G shows a different cross-sectional SPECT-CT image (upper panel) taken at the indicated time with a corresponding ROI analysis (lower panel). FIG. 11H shows cross-sectional images in approximately the same plane as represented in FIG. 11G, with the upper left and upper right panels showing SPECT-CT images and the lower left and lower right panels showing corresponding ROI analyses.

FIG. 11I is a plot of the uptake signal (ROI) versus time after injection for data generated in the experiments represented in FIGS. 11A-11H.

FIG. 12A concerns experiments involving treatment with hAnnA1 conjugates to extend survival of immunocompromised mice with the human NCI H209 metastatic tumor model. To generate the data shown in the Kaplan-Meier survival plot, R2G2 mice (n=3 per group) were intravenously injected with NCI H209 cells (1×10⁶) and treated with hAnnA1-CMD-cisplatin (7.5 μg, 1.5 μg/kg equivalent of cisplatin) or hAnnA1-I¹²⁵ (8 μg, specific activity 6.5-7.4 μCi/kg) on days 41, 48, 55, 62, 77, 90, and 98 Animals treated with either immunoconjugate showed no side effects. The arrow denotes start of the treatment. (B)

FIG. 12B includes 8 photos (labeled “a”-“g”). These photos show the results of radioimmunotherapy with ¹²⁵I-mAnnA in metastatic mammary tumors in rat lungs. Hematoxylin and eosin staining of tumor sections at (a) 1, (b) 3, (c) 5, and (d-g) 7 days after i.v. injection of ¹²⁵I-mAnnA1 (10 μg). (h) High power image of tumor 5 days after control rats injected iv with ¹²⁵I-IgG (10 μg). Scale bar=10 μm for a-d and h, =100 μm for e-g. Arrowheads indicate mitotic figures. L: adjacent normal lung tissue.

FIGS. 13A-13D represent data from experiments demonstrating that transvascular pumping enhances the potency of VEGF antibodies. In these experiments, antibodies to VEGF (mVEGF) were conjugated to mAnnA1 antibodies and injected into mice implanted mammary tumor spheroids in an IVM EO tumor model system. The photos in FIG. 13A shows antibody uptake was assessed via image capture at indicated times. FIG. 13B is a histogram showing the fluorescent intensity measured for each conjugate. FIG. 13C contains photos showing the response of the tumor to treatment with the bifunctional dual antibody conjugate compared to animals injected intravenously with unconjugated mVEGF or untreated (control); tumor size was assessed over a course of 14 days. FIG. 13D is a rraph of relative tumor size for each dose of conjugate, including control versus days post-treatment.

FIGS. 14A and 14B show data demonstrating that conjugating Herceptin to mAnnA1 boosts therapeutic potency. FIG. 14A shows several photos of GFP-tagged human BT474 breast tumors grown in IVM model in mice that were treated as indicated and observed over 14 days.

FIG. 14B is a graph of relative tumor size for each dose of conjugate, including control, versus days post-treatment.

FIGS. 15A-15C show that low dose precision delivery enables acute inhibition of fibrotic signaling pathway in lung and prophylactic inhibition of lung fibrosis. To generate the data presented in these figures, rats received intratracheal injections of bleomycin and, concurrently, i.v. injections of either mTGF-β or mAPP2:mTGF-β at the indicated doses. FIG. 15A shows that targeted lung delivery of mAPP2-mTGF-β inhibits pSMAD signaling. Specifically, this immunoblot shows pSmad2 and Smad2 from whole mouse lung lysates 6 hr following acute lung injury concurrent with i.v. injection of mTBG-β alone or conjugated to mAPP2. FIG. 15B shows six photos of lungs that were removed and processed with Trichrome stain to examine collagen deposition 2 weeks later. FIG. 15C is a morphometric analysis of collagen signals from the lungs of rats treated with bleomycin, showing enhanced attenuation of fibrosis with lung-specific targeting of TGF-β antibody. * p<0.05; ** p<0.005; ***p<0.001.

FIGS. 16A-16C show that low dose precision delivery and therapy is effective in a lung fibrosis model. FIG. 16A presents data for rats that received IT bleomycin and 12 days later, i.v. injections of either TGF-β blocking antibodies alone or conjugated to mAPP2 (both at 0.1 mg/kg). Lungs were removed 1 week later and processed for H&E and Trichrome staining. Trichrome blue shows collagen. All micrographs are at the same magnification. FIG. 16B is a histogram of whole lung collagen levels determined by Sircol biochemical assay in experiments where rats were treated as described above. (*)=p<0.05; by ranked ANOVAs with the Tukey post hoc test. FIG. 16C is a photo of an immunoblot of lung tissue from rats treated as described above that were then processed for blotting with antibodies specific for pSmad2, Smad, smooth muscle actin (SMA), and beta actin (loading control).

FIG. 17A-17D represent data from experiments to test the effects of a single injection of 1D11:r833 on bleomycin-induced inflammation and lung collagen content. FIG. 17A shows the study design for this a 3-day rat model. On day 0, animals were challenged with bleomycin (2 U/kg, i.t.). Respective groups received treatment with 1D11:r833 or vehicle 1 hr prior to bleomycin challenge and were sacrificed on day 3. BALF and lungs were harvested at each timepoint. The BALF was analyzed for inflammation and the lung homogenate subjected to Sircol assays for lung collagen estimation. FIG. 17B shows the effect of 1D11:r833 on total neutrophils. FIG. 17C shows the effects on leukocytes. FIG. 17D shows the effects on lung collagen content.

FIGS. 18A-18E show the dose response of 1D11:r833 as a preventative treatment in a bleomycin-induced pulmonary injury model. FIG. 18A shows the effect of 1D11:r833 pretreatment on total neutrophils, FIG. 18B shows the effects of this treatment on leukocytes from BALF samples, and FIG. 18C shows the effects on collagen content analyzed from lung homogenate. FIG. 18D is a Western analysis of TGF-β downstream signaling from lung homogenate samples harvested on day 3 following bleomycin challenge. FIG. 18E is an analysis of the indicated cytokines present in BALF samples.

FIGS. 19A-19G show lung-specific retargeting of PAMAM-based dendritic nanoparticles conjugated with antibodies specific to aminopeptidase 2 (mAPP2), a protein concentrated in lung vascular EC caveolae. FIG. 19A shows SPECT-CT imaging of ¹²⁵I-labeled but untargeted dendrimers G5 PAMAM. FIG. 19B shows SPECT-CT imaging of ¹²⁵I-labeled, mAPP2-targeted dendrimers G5 PAMAM. FIG. 19C shows SPECT-CT imaging of ¹²⁵I-labeled but untargeted dendrimers G5 PAMAM to various tissues. FIG. 19D shows SPECT-CT imaging of ¹²⁵I-labeled, mAPP2-targeted dendrimers G5 PAMAM to lung tissue. FIG. 19E shows SPECT-CT imaging of ¹²⁵I-labeled, mAPP2-targeted dendrimers G4 PAMAM at indicated times after i.v. injection. FIG. 19F shows SPECT-CT imaging of ¹²⁵I-labeled, untargeted dendrimers G4 PAMAM at indicated times after i.v. injection. Note these were not cloaked (not PEGylated). FIG. 19G is a schematic representation of generation-dependent physical and structural properties of PAMAM dendrimers. Adapted from Klajnert, B. and Bryszewska. M. Acta Biochim Pol 48:199-208 (2002).

FIGS. 20A-20C show IVM rapid tumor uptake and enhanced radiotherapeutic efficacy of mAnnA1-targeted dendrimers. FIG. 20A provides several photos take from nude mice with an EO model expressing H2B-CFP (red tumor cells; left column). These animals were injected iv with 3 μg of AlexaFluor488 (green) labeled PAMAM G5 dendrimers either conjugated to mAnnA1 or control isotype matched IgG (mIgG). The IVM images (size bar 200 μm) taken before injection and 1 hr after clearly show rapid tumor uptake of only the mAnnA1-dendrimers. They targeted and crossed the tumor endothelium to flood the tumor as indicated by the accumulation of green signal (see FIGS. 20B and 20C). Mice with an EO model expressing H2B-GFP (green tumor cells) were injected iv with 3 μg of ¹²⁵I-dendrimers conjugated to mIgG or linked to mAnnA1. FIG. 20B shows several photos of fluorescent IVM at low magnification on the days indicated post-treatment, which show the radioimmunotherapy was effective only with linkage to caveolae-targeting mAnnA1. Size bar, 50 μm. FIG. 20C show growth curves of tumors, demonstrating significantly enhanced radiotherapy with mAnnA1 (n=3 per data point) conjugation. Note tumor growth in the mice treated with control IgG radiodendrimer was not that different from the untreated control mice whereas the mAnnA1 radiodendrimer caused significant tumor regression.

### DETAILED DESCRIPTION OF THE INVENTION

A description of preferred embodiments of the invention follows. The invention is based on precision delivery mediated by the caveolae pumping system as a means to enhance therapeutic efficacy at ultra low doses.

### Vascular Endothelium and Tissue Accessibility

As described previously, plasmalemmal vesicles called caveolae are abundant on the endothelial cell surface, function in selective endocytosis and transcytosis of nutrients, and provide a means to enter endothelial cells (endocytosis) and/or to penetrate the endothelial cell barrier (transcytosis) for delivery to underlying tissue cells. To overcome the poor delivery efficiency that hampers the effectiveness of targeted medicines engineered to selectively seek and destroy diseased cells, here we focus on the vascular endothelial cell surface that is in immediate and intimate contact with the circulating blood. This vascular endothelial cell surface provides an inherently accessible, and thus targetable, surface on diseased tissues and organs. Targeting the caveolae pumping system is a viable means to breach the vascular endothelial barrier to improve precision drug delivery and enhance the potency of systemically administered drugs to treat diseases, including for example but not limited to solid cancers and pulmonary fibrosis.

### Delivery of Agents

In certain embodiments of the invention, a targeted drug conjugate composition comprising a carrier and targeted drug conjugate further comprising an active ingredient conjugated to a targeting agent is precisely delivered to target disease tissue as a result of the targeting agent specifically binding to an extracellular domain of a protein displayed on the outer surface of a cell membrane of a vascular endothelial cell. It is believed that the therapeutic activity of active agents will be enhanced because of precision delivery directly into targeted diseased tissue. It is further believed that using a highly efficient and rapid transvascular pathway to deliver agents directly into target tissue will concentrate such agents and thereby boost activity even at ultra low doses as a result of precision delivery.

In certain embodiments, the targeted drug conjugate composition comprising an active ingredient that is present in an amount at least about 10-fold less than an effective amount of the active ingredient present in an untargeted drug composition.

In certain embodiments, the targeted drug conjugate composition is used to treat a disease or condition tissue arising from non-hematologic cancers, an infection, inflammation, fibrosis, acute injury, infarction or other pathological malfunction. The term ‘non-hematologic cancers’ as used herein refers to solid cancers an includes sarcomas, carcinomas, lymphomas and metastatic lesions. In representative embodiments, non-hematologic cancers that can be targeted include brain, breast, lung, kidney, prostate, ovarian, head and heck, and liver tumors and lesions arising from metastases.

An agent that specifically binds to a targeted protein, as the term is used herein, is an agent that preferentially or selectively binds to that targeted protein. While certain degree of non-specific interaction may occur between the agent that specifically binds and the targeted protein, nevertheless, specific binding, may be distinguished as mediated through specific recognition of the targeted protein, in whole or part. Typically, specific binding results in a much stronger association between the agent and the targeted protein than between the agent and other proteins, e.g., other vascular proteins. The affinity constant (Ka, as opposed to Kd) of the agent for its cognate is at least 10⁶ or 10⁷, usually at least 10⁸, alternatively at least 10⁹, alternatively at least 10¹⁰, or alternatively at least 10¹¹M. It should be noted, also, that “specific” binding may be binding that is sufficiently site-specific to effectively be “specific”: for example, when the degree of binding is greater by a higher degree (e.g., equal to or greater than 10-fold, equal to or greater than 20-fold, or even equal to or greater than 100-fold), the binding may become functionally equivalent to binding solely to the targeted protein at a particular location: directed and effective binding occurs with minimal or no delivery to other tissues. Thus, the amount that is functionally equivalent to specific binding can be determined by assessing whether the goal of effective delivery of agents is met with minimal or no binding to other tissues.

In a particular embodiment, the agent that specifically binds the targeted protein is or comprises an antibody or fragment of an antibody (e.g., Fab=fragments). Representative antibodies include commercially available antibodies (as listed in Linscotts Directory). Alternatively, the agent is or comprises another agent that specifically binds to a targeted protein (a specific binding partner). Representative specific binding partners include an antigen-binding fragment, a receptor, a ligand-binding receptor fragment, a receptor ligand, natural ligands, peptides, small molecules (e.g., inorganic small molecules, organic small molecules, derivatives of small molecules, composite small molecules); aptamers; cells, including modified cells; vaccine-induced or other immune cells; nanoparticles (e.g., lipid or non-lipid based formulations); lipids; lipoproteins; lipopeptides; lipid derivatives; liposomes; modified endogenous blood proteins used to carry chemotherapeutics; a protein (e.g., a recombinant protein or a recombinant modified protein) a carrier protein (e.g., albumin, modified albumin); a lytic agent; a small molecule; other nanoparticles (e.g., albumin-based nanoparticles); transferrins; immunoglobulins; multivalent antibodies; lipids; lipoproteins; liposomes; an altered natural ligand; a gene or nucleic acid; RNA or siRNA; a viral or non-viral gene delivery vector; a prodrug; or a promolecule.

The agent can also comprise a first component that binds to the targeted protein, as described above, and a second component, that is an active component (e.g., a therapeutic agent or imaging agent, as described in detail below). The agent can be administered by itself, or in a composition (e.g., a pharmaceutical or physiological composition) comprising the agent. It can be administered either in vivo (e.g., to an individual) or in vitro (e.g., to a tissue sample). The methods of the invention can be used not only for human individuals, but also are applicable for veterinary uses (e.g., for other mammals, including domesticated animals (e.g., horses, cattle, sheep, goats, pigs, dogs, cats, etc.) and non-domesticated animals.

The agent can be administered by itself, or in a composition (e.g., a physiological or pharmaceutical composition) comprising the agent. For example, the therapeutic targeting agent can be formulated together with a physiologically acceptable carrier or excipient to prepare a pharmaceutical composition. The carrier and composition can be sterile. The formulation should suit the mode of administration.

If desired, non-specific background and/or scavenger uptake of agents by reticulo-endothelial system (primarily liver and spleen) may be reduced by overwhelming the system by inhibition and/or competitions with various reagents, including, for example, immunoglobulins, proteins or protein fragments, starches or hydroxyethylstarches, albumins, modified albumins, or other agents. Such agents can be administered prior to, or concurrently with, the agents of the invention.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active agents. The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc.

Methods of introduction of these compositions include, but are not limited to, intradermal, intramuscular, intraperitoneal, intraocular, intravenous, subcutaneous, topical, oral and intranasal. Other suitable methods of introduction can also include rechargeable or biodegradable devices, particle acceleration devises (gene guns) and slow release polymeric devices. If desired, the compositions can be administered into a specific tissue, or into a blood vessel serving a specific tissue (e.g., the carotid artery to target brain). The pharmaceutical compositions can also be administered as part of a combinatorial therapy with other agents, either concurrently or in proximity (e.g., separated by hours, days, weeks, months). The activity of the compositions may be potentiated by other agents administered concurrently or in proximity.

The composition can be formulated in accordance with the routine procedures as a pharmaceutical composition adapted for administration to human beings or animals. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., which are, if desired, sterilized or mixed with auxiliary agents, e.g., preservatives, stabilizers, wetting agents, buffers or salts for influencing osmotic pressure, etc. The agent may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

Agents described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

### Therapy

In one embodiment of the invention, methods are available for treating a disease or condition, such as but not limited to non-hematologic cancers or other pathologies in an individual, by administering a targeted therapeutic agent. The term, treatment as used herein, can refer to ameliorating symptoms associated with the non-hematologic cancer, an infection, inflammation, fibrosis, acute injury, infarction or other pathological malfunction; to reducing, preventing or delaying metastasis of the neoplasms such as cancer; to reducing the number, volume, and/or size of one or more solid tumors; and/or to lessening the severity, duration or frequency of symptoms of a disease or pathology. In these methods, a targeted drug composition is used. A targeted drug composition, as used herein, refers to a composition comprising a carrier and targeted drug conjugate that comprises an active ingredient conjugated to a targeting agent. The active ingredient is a therapeutic agent, which is present in the composition in an amount that is at least about 10-fold or 100-fold less than when an effective amount of the therapeutic agent is present in an untargeted composition used to treat a disease or condition amenable to treatment by the therapeutic agent. Further, the targeting agent specifically binds to an extracellular domain of a protein displayed on an outer surface of a cell membrane of the cell.

In another embodiment of the invention, methods are available for treating an infection, inflammation, fibrosis, acute injury, infarction or other pathological malfunction. Representative additional conditions which can be treated using the methods described herein include angiogenesis or the development of other neovasculature, atherosclerosis, diabetes and related sequelae, macular degeneration, heart disease (e.g., from ischemia), emphysema, chronic obstructive pulmonary disease, myocarditis, pulmonary and systemic hypertension and their sequelae, infection, and other conditions relating to expression of inflammatory-, angiogenesis- or neovasculature-related proteins, such as those described herein. Expression of angiogenesis-related proteins is a contributor to a variety of malignant, ischemic, inflammatory, infectious and immune disorders. Thus, the methods are similarly applicable to such conditions, which are collectively referred to herein as “pathology”.

In certain embodiments, the targeted drug composition comprises an antibody that specifically binds a targeted protein, as described herein (e.g., Annexin A1, Aminopeptidase 2). An antibody is an immunoglobulin molecule obtained by in vitro or in vivo generation of the humoral response, and includes both polyclonal and monoclonal antibodies. The term also includes genetically engineered forms such as chimeric antibodies (e.g., humanized murine antibodies), heteroconjugate antibodies (e.g., bispecific antibodies), and recombinant single chain Fv fragments (scFv). The term “antibody” also includes multivalent antibodies as well as antigen binding fragments of antibodies, such as Fab′, F(ab′)₂, Fab, Fv, rIgG, and, inverted IgG, as well as the variable heavy and variable light chain domains. An antibody immunologically reactive with a targeted protein can be generated in vivo or by recombinant methods such as selection of libraries of recombinant antibodies in phage or similar vectors. See, e.g., Huse et al. (1989) Science 246:1275-1281; and Ward, et al. (1989) Nature 341:544-546; and Vaughan et al. (1996) Nature Biotechnology, 14:309-314. An antigen-binding fragment includes any portion of an antibody that binds to the targeted protein. An antigen-binding fragment may be, for example, a polypeptide including a CDR region, or other fragment of an immunoglobulin molecule that retains the affinity and specificity for the targeted protein.

In another embodiment, the targeting agent comprises a member of a high-affinity binding pair high-affinity binding pair, optionally a molecule selected from the group consisting of an antibody, an antigen-binding antibody fragment, a receptor, a ligand-binding receptor fragment, a receptor ligand, a small molecule, and an aptamer.

In one representative therapeutic targeting agent, a multivalent antibody is used. One moiety of the multivalent antibody can serve as the targeting agent component, and a second moiety of the multivalent antibody can serve as the active agent component.

In another embodiment, the targeting agent component is linked to the active (or therapeutic) agent component. For example, they can be covalently bonded directly to one another. Where the two are directly bonded to one another by a covalent bond, the bond may be formed by forming a suitable covalent linkage through an active group on each moiety. For instance, an acid group on one compound may be condensed with an amine, an acid or an alcohol on the other to form the corresponding amide, anhydride or ester, respectively. In addition to carboxylic acid groups, amine groups, and hydroxyl groups, other suitable active groups for forming linkages between a targeting agent component and an active agent component include sulfonyl groups, sulfhydryl groups, and the haloic acid and acid anhydride derivatives of carboxylic acids.

In other embodiments, the targeting agent component and the therapeutic agent component may be covalently linked to one another through an intermediate linker. The linker advantageously possesses two active groups, one of which is complementary to an active group on the targeting agent component, and the other of which is complementary to an active group on the active agent component. For example, where the both possess free hydroxyl groups, the linker may suitably be a diacid, which will react with both compounds to form a diether linkage between the two residues. In addition to carboxylic acid groups, amine groups, and hydroxyl groups, other suitable active groups for forming linkages between pharmaceutically active moieties include sulfonyl groups, sulfhydryl groups, and the haloic acid and acid anhydride derivatives of carboxylic acids.

Suitable linkers are set forth in the table below.

| FIRST ACTIVE | SECOND ACTIVE | |
|---|---|---|
| GROUP | GROUP | SUITABLE LINKER |
| Amine | Amine | Diacid |
| Amine | Hydroxy | Diacid |
| Hydroxy | Amine | Diacid |
| Hydroxy | Hydroxy | Diacid |
| Acid | Acid | Diamine |
| Acid | Hydroxy | Amino acid, hydroxyalkyl acid, |
| | | sulfhydrylalkyl acid |
| Acid | Amine | Amino acid, hydroxyalkyl acid, |
| | | sulfhydrylalkyl acid |

Suitable diacid linkers include oxalic, malonic, succinic, glutaric, adipic, pimelic, suberic, azelaic, sebacic, maleic, fumaric, tartaric, phthalic, isophthalic, and terephthalic acids. While diacids are named, the skilled artisan will recognize that in certain circumstances the corresponding acid halides or acid anhydrides (either unilateral or bilateral) are preferred as linker reprodrugs. A preferred anhydride is succinic anhydride. Another preferred anhydride is maleic anhydride. Other anhydrides and/or acid halides may be employed by the skilled artisan to good effect.

Suitable amino acids include butyric acid, 2-aminoacetic acid, 3-aminopropanoic acid, 4-aminobutanoic acid, 5-aminopentanoic acid, 6-aminohexanoic acid, alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. Again, the acid group of the suitable amino acids may be converted to the anhydride or acid halide form prior to their use as linker groups.

Suitable diamines include 1, 2-diaminoethane, 1,3-diaminopropane, 1,4-diaminobutane, 1,5-diaminopentane, 1,6-diaminohexane. Suitable aminoalcohols include 2-hydroxy-1-aminoethane, 3-hydroxy-1-aminoethane, 4-hydroxy-1-aminobutane, 5-hydroxy-1-aminopentane, 6-hydroxy-1-aminohexane.

Suitable hydroxyalkyl acids include 2-hydroxyacetic acid, 3-hydroxypropanoic acid, 4-hydroxybutanoic acid, 5-hydroxypentanoic acid, 5-hydroxyhexanoic acid.
The person having skill in the art will recognize that by selecting the components of the targeting agent component and active agent component having suitable active groups, and by matching them to suitable linkers, a broad palette of inventive compounds may be prepared within the scope of the present invention.

Moreover, the various linker groups can be designated either “weak” or “strong” based on the stability of the covalent bond which the linker functional group will form between the spacer and either the polar lipid carrier or the biologically active compound. The weak functionalities include, but are not limited to phosphoramide, phosphoester, carbonate, amide, carboxyl-phosphoryl anhydride, ester and thioester. The strong functionalities include, but are not limited to ether, thioether, amine, sterically hindered amides and esters. The use of a strong linker functional group between the spacer group and the biologically-active compound will tend to decrease the rate at which the compound will be released at the target site, whereas the use of a weak linker functional group between the spacer group and the compound may act to facilitate release of the compound at the target site.

Enzymatic release is also possible, but such enzyme-mediated modes of release will not necessarily be correlated with bond strength in such embodiments of the invention. Spacer moieties comprising enzyme active site recognition groups, such as spacer groups comprising peptides having proteolytic cleavage sites therein, are envisioned as being within the scope of the present invention. In certain embodiments, the linker moiety includes a spacer molecule that facilitated hydrolytic or enzymatic release of the active agent component from the targeting agent component. In particularly preferred embodiments, the spacer functional group is hydrolyzed by an enzymatic activity found in the target vascular tissue, preferably an esterase.

The active therapeutic agent component, which is linked to the targeting agent component, can be or comprise any agent that achieves the desired therapeutic result, including agents such as but not limited to the following, which can be used as an active agent component for a targeted therapeutic agent, as appropriate: a radionuclide (e.g., I¹²⁵, I¹²³, I¹²⁴, I¹³¹ or other radioactive agent, including but not limited to Lu¹⁷⁷, Pb²¹², Tc⁹⁹, Zr⁸⁹, Y⁹⁰, Ac²²⁵); a chemotherapeutic agent (e.g., an antibiotic, antiviral or antifungal, including but not limited to toxins such as the epipolythiodioxopiperazine (ETP) class of fungal toxins); inhibitors, such as but not limited to chemotherapeutic inhibitors (e.g. alkylating agents, anthracyclines, cytoskeletal disruptors (taxanes), epothilones, histone deacetylase inhibitors, histone methyltransferase inhibitors, inhibitors of topoisomerase I, inhibitors of topoisomerase II, kinase inhibitors, nucleotide analogs and precursor analogs, peptide antibiotics, and platinum-based agents; an immune stimulatory agent (e.g., a cytokine); an anti-neoplastic agent: an anti-inflammatory agent; a pro-inflammatory agent; a pro-apoptotic agent (e.g., peptides or other agents to attract immune cells and/or stimulate the immune system); a pro-coagulant; a toxin (e.g., ricin, enterotoxin, LPS); an antibiotic; a hormone; a protein (e.g., a recombinant protein or a recombinant modified protein); an immune stimulatory agent, a lytic agent, a carrier protein (e.g., albumin, modified albumin); an enzyme; another protein (e.g., a surfactant protein, a clotting protein); a lytic agent; a small molecule (e.g., inorganic small molecules, organic small molecules, derivatives of small molecules, composite small molecules); aptamers; cells, including modified cells; vaccine-induced or other immune cells; nanoparticles (e.g., lipid or non-lipid based formulations, albumin-based formulations, polymers); transferrins; immunoglobulins; multivalent antibodies; lipids; lipoproteins; lipopeptides; liposomes; lipid derivatives; an natural ligand; and altered protein (e.g., albumin or other blood carrier protein-based delivery system, modified to increase affinity for the targeted protein; orosomucoid); an agent that alters the extracellular matrix of the targeted cell; an agents that inhibits growth, migration or formation of vascular structures (for a therapeutic targeting agent); an agent that enhances or increases growth, migration or formation of vascular structures (for an neovasculature targeting agent); a gene or nucleic acid (e.g., an antisense oligonucleotide RNA; siRNA); viral or non-viral gene delivery vectors or systems; or a prodrug or promolecule; a microtubule-targeted agent (e.g. maytansine or derivatives and and analogs thereof); natural or synthetic toxins, including but not limited to alpha-amanitin derivatives.

A “chemotherapeutic agent” is a chemical compound useful in the treatment of cancer. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaII (see, e.g., Nicolaou et al., Angew. Chem Intl. Ed. Engl., 33: 183-186 (1994)); CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin (actinomycin D), authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCI liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, valrubicin, zinostatin, zorubicin; anti-metabolites such as methotrexate, pemetrexed, gemcitabine (GEMZAR®), tegafur (UFTORAL®), capecitabine (XELODA®), an epothilone, and 5-fluorouracil (5-FU); combretastatin; folic acid analogues such as denopterin, methotrexate, pteropterin, trimetrexate; purine analogs such as azathioprine, cladribine, fludarabine, 6-mercaptopurine, thiamiprine, thioguanine; pyrimidine analogs such as ancitabine, azacitidine, 6-azauridine, capecitabine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK®, polysaccharide complex (JHS Natural Products, Eugene, Oreg.); razoxane; rhizoxin; sizofuran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE®, FILDESIN®); dacarbazine (Temozolomide; mannomustine; mitobronitol; mitolactol; pipobroman; gacytosine; arabinoside (“Ara-C”); thiotepa; taxoid, e.g., taxotere, paclitaxel (TAXOL®), albumin-engineered nanoparticle formulation of paclitaxel (ABRAXANE™), and docetaxel (TAXOTERE®, Rhome-Poulene Rorer, Antony, France); chloranbucil; 6-thioguanine (tioguanine); mercaptopurine; methotrexate; platinum agents such as cisplatin, oxaliplatin (e.g., ELOXATIN®), and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); tubulin inhibitors, such as thiol-containing maytansinoid, including mertansine, also called DM1 (and in some of its forms, emtansine), which in some embodiments include trastuzumab emtansine also known as ado-trastuzumab emtansine (Kadcyla®); etopside (VP-16); ifosfamide; mitoxantrone; leucovorin; novantrone; edatraxate; daunomycin; aminopterin; ibandronate; topoisomerase inhibitor RFS 2000; difluoromethylomithine (DMFO); retinoids such as retinoic acid, including alitretinoin, bexarotene (TARGRETIN®), tretinoin; bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R) (e.g., erlotinib (Tarceva™)); and VEGF-A that reduce cell proliferation; vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); BAY439006 (sorafenib; Bayer); SU-11248 (sunitinib, SUTENT®, Pfizer); perifosine, COX-2 inhibitor (e.g. celecoxib or etoricoxib), proteosome inhibitor (e.g. PS341); bortezomib (VELCADE®); CCI-779; tipifamib (R11577); orafenib, ABT510; Bcl-2 inhibitor such as oblimersen sodium (GENASENSE®); pixantrone; EGFR inhibitors; tyrosine kinase inhibitors such as gefitinib, imatinib, vemurafenib, and vismodegib; serine-threonine kinase inhibitors such as rapamycin (sirolimus, RAPAMUNE®); anthracyclines; famesyltransferase inhibitors such as lonafamib (SCH 6636, SARASAR®); histone deacetylase inhibitors such as vorinostat and romidepsin; hydrazines such as temozolomide; topoisomerase I inhibitors such as irinotecan and topotecan; topoisomerase II inhibitors such as tafluposide and teniposide; and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin, and pharmaceutically acceptable salts, acids or derivatives of any of the above; as well as combinations of two Or more of the above. Chemotherapeutic agents as defined herein include “anti-hormonal agents” or “endocrine therapeutics” which act to regulate, reduce, block, or inhibit the effects of hormones that can promote the growth of cancer. They may be hormones themselves, including, but not limited to: anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON.cndot.toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Raf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rIL-2; LURTOTECA® topoisomerase 1 inhibitor; ABARELIX®; Vinorelbine and Esperamicins (see U.S. Pat. No. 4,675,187), and pharmaceutically acceptable salts, acids or derivatives of any of the above; as well as combinations of two or more of the above.

For example, in one embodiment, a radionuclide or other radioactive agent can be used as the active agent component. The targeting agent component delivers the radioactive agent in a tissue-specific manner, allowing local radiation damage and resulting in radiation-induced apoptosis and necrosis throughout the solid tumor or diseased tissue including in tumor cells, stromal calls, and endothelial cells of the tumor or throughout the diseased area. Alternatively, in another embodiment, an active agent that exhibits anti-fibrotic activity can be used to treat pulmonary fibrosis. In yet another embodiment, the active agent is effective to treat acute lung injury. The targeting agent component delivers the agent in a highly precise manner directly into diseased tissue and therapeutic activity is enhanced because the ultra low dose that is administered is concentrated at the desired site where it can be most effective.

In another embodiment, antisense oligonucleotides or other agents can be used as the active agent component, to alter, and particularly to inhibit, production of a gene in a targeted tissue, such as a gene that is overexpressed in a tumor tissue (e.g., an oncogene or a gene associated with neoplasm, such as c-Jun, c-Fos, HER-2, E2F-1, RAS, FAS, NF, BRCA), or a gene that is overexpressed in angiogenesis. Alternatively, oligonucleotides or genes can be used to alter, and particularly to enhance, production of a protein in the targeted tissue, such as a gene that controls apoptosis or regulates cell growth; oligonucleotides or genes can also be used to produce a protein that is under-expressed or deleted in the targeted tissue, or to express a gene product that is directly or indirectly destructive to the neoplasm.

In a particular embodiment, an anti-fibrotic agent can be used as the active agent. Representative agents include rapamycin, troglitazone, therapeutic peptides such as Thy-1, and therapeutic antibodies such as those again TGF-β. In a further embodiment, an anti-inflammatory agent can be used as the active agent. Representative agents include a non-steroidal anti-inflammatory agent; a steroidal or corticosteroidal anti-inflammatory agent; or other anti-inflammatory agent (e.g., histamine). In other embodiments, the active agent can be an agent to alter blood pressure (e.g., a diuretic, a vasopressin agonist or antagonist, angiotensin). Alternatively, pro-inflammatory agents can be used as active agents (e.g., to enhance angiogenesis or increase development of neovasculature, as described herein).

In another particular embodiment, chemotherapeutic agents for neoplastic diseases can be used as the active agent component. Representative agents include alkylating agents (nitrogen mustards, ethylenimines, alkyl sulfonates, nitrosoureas, and triazenes), antimetabolites (folic acid analogs such as methotrexate, pyrimidine analogs, and purine analogs), natural products and their derivatives (antibiotics, alkaloids, enzymes), hormones and antagonists (corticosteroids; adrenocorticosteroids, progestins, estrogens), and other similar agents. For example, in certain embodiments, the chemotherapeutic agent can be acytotoxic or cytostatic drugs. Chemotherapeutics may also include those that have other effects on cells such as reversal of the transformed state to a differentiated state or those which inhibit cell replication. Examples of known cytotoxic agents useful in the present invention are listed, for example, in Goodman et al., “The Pharmacological Basis of Therapeutics,” Sixth Edition, A. G. Gilman et a.l, eds./Macmillan Publishing Co. New York, 1980. These include taxol, nitrogen mustards, such as mechlorethamine, cyclophosphamide, melphalan, uracil mustard and chlorambucil; ethylenimine derivatives, such as thiotepa; alkyl sulfonates, such as busulfan; nitrosoureas, such as carmustine, lomustine, semustine and streptozocin; triazenes, such as dacarbazine; folic acid analogs, such as methotrexate; pyrimidine analogs, such as fluorouracil, cytarabine and azaribine; purine analogs, such as mercaptopurine and thioguanine; vinca alkaloids, such as vinblastine and vincristine; antibiotics, such as dactinomycin, daunorubicin, doxorubicin, bleomycin, mithramycin and mitomycin; enzymes, such as L-asparaginase; platinum coordination complexes, such as cisplatin; substituted urea, such as hydroxyurea; methyl hydrazine derivatives, such as procarbazine; adrenocortical suppressants, such as mitotane; hormones and antagonists, such as adrenocortisteroids (prednisone), progestins (hydroxyprogesterone caproate, medroprogesterone acetate and megestrol acetate), estrogens (diethylstilbestrol and ethinyl estradiol), antiestrogens (tamoxifen), and androgens (testosterone propionate and fluoxymesterone).

Drugs that interfere with intracellular protein synthesis can also be used; such drugs are known to these skilled in the art and include puromycin, cycloheximide, and ribonuclease.

Most of the chemotherapeutic agents currently in use in treating cancer possess functional groups that are amenable to chemical crosslinking directly with an amine or carboxyl group of a targeting agent component. For example, free amino groups are available on methotrexate, doxorubicin, daunorubicin, cytosinarabinoside, cis-platin, vindesine, mitomycin and bleomycin while free carboxylic acid groups are available on methotrexate, melphalan, and chlorambucil. These functional groups, that is free amino and carboxylic acids, are targets for a variety of homobifunctional and heterobifunctional chemical crosslinking agents which can crosslink these drugs directly to a free amino group.

Peptide and polypeptide toxins are also useful as active agent components, and the present invention specifically contemplates embodiments wherein the active agent component is a toxin. Toxins are generally complex toxic products of various organisms including bacteria, plants, etc. Examples of toxins include but are not limited to: ricin, ricin A chain (ricin toxin), Pseudomonas exotoxin (PE), diphtheria toxin (DT), Clostridium perfringens phospholipase C (PLC), bovine pancreatic ribonuclease (BPR), pokeweed antiviral protein (PAP), abrin, abrin A chain (abrin toxin), cobra venom factor (CVF), gelonin (GEL), saporin (SAP), modeccin, viscumin and volkensin.

In a further particular embodiment, an anti-inflammatory agent can be used as the active agent. Representative agents include a non-steroidal anti-inflammatory agent; a steroidal or corticosteroidal anti-inflammatory agent; or other anti-inflammatory agent (e.g., histamine). Alternatively, pro-inflammatory agents can be used as active agents (e.g., to enhance angiogenesis or increase development of neovasculature, as described herein).

Prodrugs or promolecules can also be used as the active agent. For example, a prodrug that is used as an active agent can subsequently be activated (converted) by administration of an appropriate enzyme, or by endogenous enzyme in the targeted tissue. Alternatively, the activating enzyme can be co-administered or subsequently administered as another active agent as part of a therapeutic agent as described herein; or the prodrug or promolecule can be activated by a change in pH to a physiological pH upon administration. Representative prodrugs include Herpes simplex virus thymidine kinase (HSV TK) with the nucleotide analog GCV; cytosine deaminase ans t-fluorocytosine; alkaline phosphatase/etoposidephosphate; and other prodrugs (e.g., those described in Greco et al., J. Cell. Phys. 187:22-36, 2001; and Konstantinos et al., Anticancer Research 19:605-614, 1999; see also Connors, T. A., Stem Cells 13(5): 501-511, 1995; Knox, R. J., Baldwin, A. et al., Arch. Biochem. Biophys. 409(1):197-206, 2003; Syrigos, K. N. and Epenetos, A. A., Anticancer Res. 19(1A): 605-613, 1999; Denny, W. A., JBB 1:48-70, 2003).

In another embodiment of the invention, the targeting agent component and/or the active agent component comprises a chelate moiety for chelating a metal, e.g., a chelator for a radiometal or paramagnetic ion. In preferred embodiments, the a chelator is a chelator for a radionuclide. Radionuclides useful within the present invention include gamma-emitters, positron-emitters, Auger electron-emitters, X-ray emitters and fluorescence-emitters, with beta- or alpha-emitters preferred for therapeutic use. Examples of radionuclides useful as toxins in radiation therapy include: ²²⁵Ac, ⁸⁹Zr, ³²P, ³³P, ⁴³K, ⁴⁷Sc, ⁵²Fe, ⁵⁷Co, ⁶⁴Cu, ⁶⁷Ga, ⁶⁷Cu, ⁶⁸Ga, ⁷¹Ge, ⁷⁵Br, ⁷⁶Br, ⁷⁷Br, ⁷⁷As, ⁷⁷Br, ⁸¹Rb/^{81M}Kr, ^{87M}Sr, ⁹⁰Y, ⁹⁷Ru, ⁹⁹Tc, ¹⁰⁰Pd, ¹⁰¹Rb, ¹⁰³Pb, ¹⁰⁵Rh, ¹⁰⁹Pd, ¹¹¹Ag, ¹¹¹In, ¹¹³In, ¹¹⁹Sb, ¹²¹Sn, ¹²³I, ¹²⁵I, ¹²⁷Cs, ¹²⁸Ba, ¹²⁹Cs, ¹³¹I, ¹³¹Cs, ¹⁴³Pr, ¹⁵³Sm, ¹⁶¹Tb, ¹⁶⁶Ho, ¹⁶⁹Eu, ¹⁷⁷Lu, ¹⁸⁶Re, ¹⁸⁸Re, ¹⁸⁹Re, ¹⁹¹Os, ¹⁹³Pt, ¹⁹⁴Ir, ¹⁹⁷Hg, ¹⁹⁹Au, ²⁰³Pb, ²¹¹At, ²¹²Pb, ²¹²Bi and ²¹³Bi. Preferred therapeutic radionuclides include ¹⁸⁸Re, ¹⁸⁶Re, ²⁰³Pb, ²¹²Pb, ²¹²Bi, ¹⁰⁹Pd, ⁶⁴Cu, ⁶⁷Cu, ⁹⁰Y, ¹²⁵I, ¹³¹I, ⁷⁷Br, ²¹¹At, ⁹⁷Ru, ¹⁰⁵Rh, ¹⁹⁸Au and ¹⁹⁹Ag, ¹⁶⁶Ho, ²²⁵Ac, ⁸⁹Zr, or ¹⁷⁷Lu. Conditions under which a chelator will coordinate a metal are described, for example, by Gansow et al., U.S. Pat. Nos. 4,831,175, 4,454,106 and 4,472,509.

In one embodiment, for example, ^{99m}Tc can be used as a radioisotope for therapeutic and diagnostic applications (as described below), as it is readily available to all nuclear medicine departments, is inexpensive, gives minimal patient radiation doses, and has ideal nuclear imaging properties. It has a half-life of six hours which means that rapid targeting of a technetium-labeled antibody is desirable. Accordingly, in certain preferred embodiments, the therapeutic targeting agent includes a chelating agents for technium.

The therapeutic targeting agent can also comprise radiosensitizing agents, e.g., a moiety that increase the sensitivity of cells to radiation. Examples of radiosensitizing agents include nitroimidazoles, metronidazole and misonidazole (see: DeVita, V. T. Jr. in Harrison's Principles of Internal Medicine, p. 68, McGraw-Hill Book Co., N.Y. 1983, which is incorporated herein by reference). The therapeutic targeting agent that comprises a radiosensitizing agent as the active moiety is administered and localizes in the endothelial call and/or in any other cells of the neoplasm. Upon exposure of the individual to radiation, the radiosensitizing agent is “excited” and causes the death of the cell.

There are a wide range of moieties which can serve as chelating ligands and which can be derivatized as part of the therapeutic targeting agent. For instance, the chelating ligand can be a derivative of 1,4,7,10-tetraazacyclododecanetetraacetic acid (DOTA), ethylenediaminetetraacetic acid (EDTA), diethylenetriaminepentaacetic acid (DTPA) and 1-p-Isothiocyanato-benzyl-methyl-diethylenetriaminepentaacetic acid (ITC-MX). These chelators typically have groups on the side chain by which the chelator can be used for attachment to a targeting agent component. Such groups include, e.g., benzylisothiocyanate, by which the DOTA, DTPA or EDTA can be coupled to, e.g., an amine group of the inhibitor.

In one embodiment, the agent is an “NₓS_{y}” chelate moiety. As defined herein, the term “NₓS_{y} chelates” includes bifunctional chelators that are capable of coordinately binding a metal or radiometal and, preferably, have N₂S₂ or N₃S cores. Exemplary NₓS_{y} chelates are described, e.g., in Fritzberg et al. (1988) PNAS 85:4024-29; and Weber et al. (1990) Bioconjugate Chem. 1:431-37; and in the references cited therein. The Jacobsen et al. PCT application WO 98/12156 provides methods and compositions, i.e. synthetic libraries of binding moieties, for identifying compounds which bind to a metal atom. The approach described in that publication can be used to identify binding moieties that can subsequently be incorporated into therapeutic targeting agents.

A problem frequently encountered with the use of conjugated proteins in radiotherapeutic and radiodiagnostic applications is a potentially dangerous accumulation of the radiolabeled moiety fragments in the kidney. When the conjugate is formed using a acid- or base-labile linker, cleavage of the radioactive chelate from the protein can advantageously occur. If the chelate is of relatively low molecular weight, it is not retained in the kidney and is excreted in the urine, thereby reducing the exposure of the kidney to radioactivity. However, in certain instances, it may be advantageous to utilize acid- or base-labile linkers in the subject ligands for the same reasons they have been used in labeled proteins.

Other appropriate active agents include agents that induce intravascular coagulation, or which damage the endothelium, thereby causing coagulation and effectively infracting a neoplasm or other targeted pathology. In addition, if desired, enzymes activated by other agents (e.g., biotin, activated by avidin) can be used as active agents or as part of the therapeutic targeting agent.

The therapeutic targeting agents can be synthesized, by standard methods known in the art (e.g., by recombinant DNA technology or other means), to provide reactive functional groups that can form acid-labile linkages with, e.g., a carbonyl group of the ligand. Examples of suitable acid-labile linkages include hydrazone and thiosemicarbazone functions. These are formed by reacting the oxidized carbohydrate with chelates bearing hydrazide, thiosemicarbazide, and thiocarbazide functions, respectively. Alternatively, base-cleavable linkers, that have been used for the enhanced clearance of the radiolabel from the kidneys, can be used. See, for example, Weber et al. 1990 Bioconjug. Chem. 1:431. The coupling of a bifunctional chelate via a hydrazide linkage can incorporate base-sensitive ester moieties in a linker spacer arm. Such an ester-containing linker unit is exemplified by ethylene glycolbis(succinimidyl succinate), (EGS, available from Pierce Chemical Co., Rockford, Ill.), which has two terminal N-hydroxysuccinimide (NHS) ester derivatives of two 1,4-dibutyric acid units, each of which are linked to a single ethylene glycol moiety by two alkyl esters. One NHS ester may be replaced with a suitable amine-containing BFC (for example 2-aminobenzyl DTPA), while the other NHS ester is reacted with a limiting amount of hydrazine. The resulting hyrazide is used for coupling to the targeting agent component, forming an ligand-BFC linkage containing two alkyl ester functions. Such a conjugate is stable at physiological pH, but readily cleaved at basic pH.

Therapeutic targeting agents labeled by chelation are subject to radiation-induced scission of the chelator and to loss of radioisotope by dissociation of the coordination complex. In some instances, metal dissociated from the complex can be re-complexed, providing more rapid clearance of non-specifically localized isotope and therefore less toxicity to non-target tissues. For example, chelator compounds such as EDTA or DTPA can be infused into patients to provide a pool of chelator to bind released radiometal and facilitate excretion of free radioisotope in the urine.

In still other embodiments, a Boron addend, such as a carborane, can be used. For example, carboranes can be prepared with carboxyl functions on pendant side chains, as is well known in the art. Attachment of such carboranes to an amine functionality, e.g., as may be provided on the targeting agent component can be achieved by activation of the carboxyl groups of the carboranes and condensation with the amine group to produce the conjugate. Such therapeutic agents can be used for neutron capture therapy.

In a further embodiment, RNAi is used. “RNAi construct” is a generic term used throughout the specification to include small interfering RNAs (siRNAs), hairpin RNAs, and other RNA species which can be delivered ectopically to a cell, cleaved by the enzyme dicer and cause gene silencing in the cell. The term “small interfering RNAs” or “siRNAs” refers to nucleic acids around 19-30 nucleotides in length, and more preferably 21-23 nucleotides in length. The siRNAs are double-stranded, and may include short overhangs at each end. Preferably, the overhangs are 1-6 nucleotides in length at the 3=end. It is known in the art that the siRNAs can be chemically synthesized, or derive by enzymatic digestion from a longer double-stranded RNA or hairpin RNA molecule. For efficiency, an siRNA will generally have significant sequence similarity to a target gene sequence. Optionally, the siRNA molecules includes a 3′ hydroxyl group, though that group may be modified with a fatty acid moiety as described herein. The phrase “mediates RNAi” refers to (indicates) the ability of an RNA molecule capable of directing sequence-specific gene silencing, e.g., rather than a consequence of induction of a sequence-independent double stranded RNA response, e.g., a PKR response.

In certain embodiments, the RNAi construct used for the active agent component is a small-interfering RNA (siRNA), preferably being 19-30 base pairs in length. Alternatively, the RNAi construct is a hairpin RNA which can be processed by cells (e.g., is a dicer substrate) to produce metabolic products in vivo in common with siRNA treated cells, e.g., a processed to short (19-22 mer) guide sequences that induce sequence specific gene silencing. In a preferred embodiment, the treated animal is a human.

The RNAi constructs contain a nucleotide sequence that hybridizes under physiologic conditions of the cell to the nucleotide sequence of at least a portion of the mRNA transcript for the gene to be inhibited (i.e., the “target” gene). The double-stranded RNA need only be sufficiently similar to natural RNA that it has the ability to mediate RNAi. Thus, the invention has the advantage of being able to tolerate sequence variations that might be expected due to genetic mutation, strain polymorphism or evolutionary divergence. The number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 501C or 701C hybridization for 12-16 hours; followed by washing).

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro.

The RNAi constructs may include other modifications, such as to the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of a nitrogen or sulfur heteroatom. Modifications in RNA structure may be tailored to allow specific genetic inhibition while avoiding a general cellular response to dsRNA (a APKR-mediated response @). Likewise, bases may be modified to block the activity of adenosine deaminase. The RNAi construct may be produced enzymatically or by partial/total organic synthesis, any modified ribonucleotide can be introduced by in vitro enzymatic or organic synthesis.

Methods of chemically modifying other RNA molecules can be adapted for modifying RNAi constructs (see, for example, Heidenreich et al. (1997) Nucleic Acids Res, 25:776-780; Wilson et al. (1994) J Mol Recog 7:89-98; Chen et al. (1995) Nucleic Acids Res 23:2661-2668; Hirschbein et al. (1997) Antisense Nucleic Acid Drug Dev 7:55-61). Merely to illustrate, the backbone of an RNAi construct can be modified with phosphorothioate, phosphorodithioate, methylphosphonate, chimeric methylphosphonate-phosphodiesters, phosphoramidate, boranophosphate, phosphotriester, formacetal, 3′-thioformacetal, 5′-thioformacetal, 5′-thioether, carbonate, 5′-N-carbamate, sulfate, sulfonate, sulfamate, sulfonamide, sulfone, sulfite, sulfoxide, sulfide, hydroxylamine, methylene(methylimino) (MMI), methyleneoxy(methylimino) (MOMI) linkages, peptide nucleic acids, 5-propynyl-pyrimidine containing oligomers or sugar modifications (e.g., 2=-substituted ribonucleosides, a-configuration).

The double-stranded structure may be formed by a single self-complementary RNA strand or two complementary RNA strands. RNA duplex formation may be initiated either inside or outside the cell.

In certain embodiments, to reduce unwanted immune stimulation, the RNAi construct is designed so as not to include unmodified cytosines occurring 5′ to guanines, e.g., to avoid stimulation of B cell mediated immunosurveillance.

In certain embodiments in which the RNAi is to be delivered for local therapeutic effect, the backbone linkages can be chosen so as titrate the nuclease sensitivity to make the RNAi sufficiently nuclease resistant to be effective in the tissue of interest (e.g., the neoplasm), but not so nuclease resistant that significant amounts of the construct could escape the tissue undegraded. With the use of this strategy, RNAi constructs are available for gene silencing in the tissue of interest, but are degraded before they can enter the wider circulation.

The RNA may be introduced in an amount that allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

In certain embodiments, the subject RNAi constructs are siRNAs. These nucleic acids are around 19-30 nucleotides in length, and even more preferably 21-23 nucleotides in length, e.g., corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group.

The siRNA molecules of the present invention can be obtained using a number of techniques known to those of skill in the art. For example, the siRNA can be chemically synthesized or recombinantly produced using methods known in the art. For example, short sense and antisense RNA oligomers can be synthesized and annealed to form double-stranded RNA structures with 2-nucleotide overhangs at each end (Caplen, et al. (2001) Proc Natl Acad Sci USA, 98:9742-9747; Elbashir, et al. (2001) EMBO J, 20:6877-88). These double-stranded siRNA structures can then be directly introduced to cells, either by passive uptake or a delivery system of choice, such as described below.

In certain embodiments, the siRNA constructs can be generated by processing of longer double-stranded RNAs, for example, in the presence of the enzyme dicer. In one embodiment, the Drosophila in vitro system is used. In this embodiment, dsRNA is combined with a soluble extract derived from Drosophila embryo, thereby producing a combination. The combination is maintained under conditions in which the dsRNA is processed to RNA molecules of about 21 to about 23 nucleotides.

The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

Modification of siRNA molecules with fatty acids can be carried out at the level of the precursors, or, perhaps more practically, after the RNA has been synthesized. The latter may be accomplished in certain instances using nucleoside precursors in the synthesis of the polymer that include functional groups for formation of the linker-fatty acid moiety.

In certain preferred embodiments, at least one strand of the siRNA molecules has a 3′ overhang from about 1 to about 6 nucleotides in length, though may be from 2 to 4 nucleotides in length. More preferably, the 3′ overhangs are 1-3 nucleotides in length. In certain embodiments, one strand having a 3′ overhang and the other strand being blunt-ended or also having an overhang. The length of the overhangs may be the same or different for each strand. In order to further enhance the stability of the siRNA, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythyinidine is tolerated and does not affect the efficiency of RNAi. The absence of a 2′ hydroxyl significantly enhances the nuclease resistance of the overhang in tissue culture medium and may be beneficial in vivo.

In other embodiments, the RNAi construct is in the form of a long double-stranded RNA. In certain embodiments, the RNAi construct is at least 25, 50, 100, 200, 300 or 400 bases. In certain embodiments, the RNAi construct is 400-800 bases in length. The double-stranded RNAs are digested intracellularly, e.g., to produce siRNA sequences in the cell. However, use of long double-stranded RNAs in vivo is not always practical, presumably because of deleterious effects which may be caused by the sequence-independent dsRNA response. In such embodiments, the use of local delivery systems and/or agents which reduce the effects of interferon or PKR are preferred.

In certain embodiments, the RNAi construct is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci USA, 2002, 99:6047-52). Preferably, such hairpin RNAs are engineered in cells or in an animal to ensure continuous and stable suppression of a desired gene. It is known in the art that siRNAs can be produced by processing a hairpin RNA in the cell.

The therapeutic targeting agent, alone or in a composition, is administered in a therapeutically effective amount, which is the amount used to treat the neoplasm or to treat angiogenesis or unwanted development of neovasculature. The amount that will be therapeutically effective will depend on the nature of the neoplasm, neovasculature or angiogenesis, the extent of disease and/or metastasis, and other factors, and can be determined by standard clinical techniques. In addition, in vitro or in vivo assays may optionally be employed to help identify optimal dosage ranges. The precise dose to be employed in the formulation will also depend on the route of administration, and the seriousness of the symptoms, and should be decided according to the judgment of a practitioner and each patient=s circumstances. Effective doses may be extrapolated from dose-response curves derived from in vitro or animal model test systems.

### Tissue Engineering

Because certain proteins have been identified as being prevalent on tumor endothelium, as described herein, methods are now available to create cell types in culture that are more similar to those in vivo. (See, e.g., Engelmann, K. Et al., Exp Ehye Res (2004) 78(3):573-8; Kirkpatrick, C. J. et al., biomol. Eng. (2002): 19(2-6):211-7; Nugent, H. M. and Edelman, E. R., Circ. Res. (2003) 92(10):1068-780). Tumor cells in vitro that are more similar to those in vivo, by virtue of producing similar panels of proteins on the endothelial surface, provide a better tool for assessing agents that may be useful in therapies such as the therapies described herein. Cells can be modified, for example, by incorporation of nucleic acids or vectors expressing proteins that are produced in excess in neoplasms, compared to expression in normal cells. Such modified cells allow more accurate assessment of effects of a potential therapeutic agent on neoplasm cells.

### Antibodies of the Invention

In another aspect, the invention provides antibodies to certain targeted proteins, that can be used, for example, in the methods of the invention. The term, Aantibody, @ is described above. The invention provides polyclonal and monoclonal antibodies that bind to a targeted protein. The term “monoclonal antibody” or “monoclonal antibody composition”, as used herein, refers to a population of antibody molecules that contain only one species of an antigen binding site capable of immunoreacting with a particular epitope of the targeted protein. Polyclonal antibodies can be prepared as described above by immunizing a suitable subject with a desired immunogen, e.g., the targeted protein or a fragment or derivative thereof. The antibody titer in the immunized subject can be monitored over time by standard techniques, such as with an enzyme linked immunosorbent assay (ELISA) using immobilized polypeptide. If desired, the antibody molecules directed against the targeted protein can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein (1975) Nature, 256:495-497, the human B cell hybridoma technique (Kozbor et al. (1983) Immunol. Today, 4:72), the EBV-hybridoma technique (Cole et al. (1985), Monoclonal Antibodies and Cancer Therapy, Alan R. Liss, Inc., pp. 77-96) or trioma techniques. The technology for producing hybridomas is well known (see generally Current Protocols in Immunology (1994) Coligan et al. (eds.) John Wiley & Sons, Inc., New York, N.Y.). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well-known protocols used for fusing lymphocytes and immortalized cell lines can be applied for the purpose of generating a monoclonal antibody to a targeted protein (see, e.g., Current Protocols in Immunology, supra; Galfre et al. (1977) Nature, 266:55052; R. H. Kenneth, in Monoclonal Antibodies: A New Dimension In Biological Analyses, Plenum Publishing Corp., New York, N.Y. (1980); and Lerner (1981) Yale J. Biol. Med., 54:387-402. Moreover, the ordinarily skilled worker will appreciate that there are many variations of such methods that also would be useful.

Alternative to preparing monoclonal antibody-secreting hybridomas, a monoclonal antibody to a targeted protein can be identified and isolated by screening a recombinant combinatorial immunoglobulin library (e. g., an antibody phage display library) with the targeted protein, to thereby isolate immunoglobulin library members that bind to the targeted protein. Kits for generating and screening phage display libraries are commercially available (e.g., the Pharmacia Recombinant Phage Antibody System, Catalog No. 27-9400-01; and the Stratagene SurfZAPJ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology, 9:1370-1372; Hay et al. (1992) Hum. Antibod. Hybridomas, 3:81-85; Huse et al. (1989) Science, 246:1275-1281; Griffiths et al. (1993) EMBO J., 12:725-734.

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

In general, antibodies of the invention (e.g., a monoclonal antibody) can be used in the methods of the invention. For example, an antibody specific for a targeted protein can be used in the methods of the invention to image a neoplasm, in order to evaluate the abundance and location of the neoplasm. Antibodies can thus be used diagnostically to, for example, determine the efficacy of a given treatment regimen, by imaging before and after the treatment regimen.

The invention will be further described by reference to the following detailed examples. These Examples are in no way to be considered to limit the scope of the invention in any manner.

### EXAMPLES

The proteomic mapping of vascular endothelium and its caveolae, reveals tissue-specific delivery targets that enable precision drug delivery to the site of desired pharmacologic activity. Specific targeted agents can now penetrate rapidly and actively into a single diseased tissue to increase therapeutic potency at ultra-low doses as the direct result of improved precision drug delivery. Targeting vascular endothelial cell proteins expressed on the outer surface of the cellular membrane permits precision delivery to, treatment of, and imaging of diseased tissue in vivo.

### Example 1

### Materials and Methods

Materials: Monoclonal antibodies to mouse VEGF (B20-4.1.1, G6-31) were provided by Dr. Napoleone Ferrara (Genentech, San Francisco, Calif.). Herceptin (trastuzumab), doxorubicin, and Taxotere (doxetaxel) were obtained via UCSD pharmacy. Mouse IgGs were obtained from Southern Biotech (Birmingham, Ala.). VEGF antibodies (B20-4.1.1 and G6-31) were a gift from Genentech (South San Francisco, Calif.). mAnnA1 and mAPP2 antibodies were made as described previously (Oh et al., 2007; Oh et al., 2004; Oh et al., 2014). Cisplatin was obtained from Sigma-Aldrich (St. Louis, Mo.), as were other chemicals and reagents unless otherwise noted.

Animals. All animal experiments were performed in accordance with IACUC guidelines at SKCC and PRISM. Female nu/nu athymic nude, C57b6 and FVB mice from either Charles River Laboratories (Wilmington, Mass.) or Jackson Laboratories (Bar Harbor, Me.) were used for the dorsal skinfold implantations and for donor tissues. We used mice that were >25 g for both the chambered mice and the donor tissues.

Fluorescent Tumor Cell Lines. All cell lines were grown at 37° C. in 5% CO₂ in air. N202 (gift from Joseph Lustgarten, Mayo Clinic, Scottsdale, Ariz.) and Lewis Lung Carcinoma (LLC1, Cat # CRL-1642—ATCC, Manassas, Va.) cells were maintained in DMEM High Glucose supplemented with L-Glutamine (2 mM), Penicillin (100 U/ml), Streptomycin (100 U/ml), Sodium Pyruvate (1 mM) (Invitrogen, Carlsbad, Calif.) and 10% heat inactivated FBS (Omega Scientific, Tarzana, Calif.). BT-474 (Cat # HTB-20-ATCC, Manassas, Va.) were maintained in Hybridoma-SFM supplemented with L-Glutamine (2 mM), Penicillin (100 U/ml), Streptomycin (100 U/ml), Sodium Pyruvate (1 mM) (Invitrogen, Carlsbad, Calif.) and 10% heat inactivated FBS (Omega Scientific, Tarzana, Calif.). TRAMPC2 (Cat # CRL-2731-ATCC, Manassas, Va.) cells were maintained in Dulbecco's modified Eagle's medium with 4 mM L-glutamine adjusted to contain 1.5 g/L sodium bicarbonate and 4.5 g/L glucose supplemented with 0.005 mg/ml bovine insulin and 10 nM dehydroisoandrosterone, 90%; fetal bovine serum, 5%; Nu-Serum IV, 5%. The histone H2B-GFP was subcloned into the SalI/HpaI sites in the LXRN vector (Clontech, Palo Alto, Calif.) using SalI and blunted NotI sites from the BOSH2BGFPN1 vector (Kanda et al., 1998). The monovalent cherry (mCherry) vector was created from the H2B-GFP vector by cloning the mCherry gene (a kind gift from Dr. Roger Tsien, UCSD) to replace the GFP gene. GP2-293 cells were infected with VSV and the H2B-GFP or H2B-mCherry containing virus to produce viable virus. N202, BT-474, TRAMPC2 and LLC1 cells were transduced with the viable virus to stably incorporate the H2B-GFP or H2B-mCherry gene. The transduced cells were FACs sorted twice to ensure 100% of the cells stably expressed the H2B-GFP or H2B-mCherry protein.

Tumor Models. We used the classic IVM tumor model with modifications (Asaishi et al., 1981; Endrich et al., 1980; Vajkoczy et al., 2000). The mice, usually athymic nude mice (25-30 g body weight), were anesthetized (7.3 mg ketamine hydrochloride and 2.3 mg xylazine per 100 g body weight, intraperitoneal injection) and placed on a heating pad. A titanium frame was placed onto the dorsal skinfold of mice to sandwich the extended double layer of skin. A 15 mm diameter full-thickness circular layer of skin was then excised. The superficial fascia on top of the remaining skin was carefully removed to expose the underlying muscle and subcutaneous tissue which was then covered with another titanium frame with a glass coverslip to form the window chamber. After a recovery period of 1-2 days, tumor spheroids were implanted.

Tumor spheroids were formed by plating 50,000 cells onto 1% agar-coated 96-well non-tissue culture treated flat bottom dishes (20 μl cells in 100 μl medium) and centrifuging 4 times at 2000 rpm for 15 min, rotating the dish after every centrifugation. The cells were incubated an additional 3-7 days (depending on cell type) at 37° C. in 5% CO₂ in air to form tight spheroids. BT-474 cells required 500,000 cells in the presence of Matrigel (BD Bioscience, San Diego) (1:1 cell volume dilution) to form spheroids in culture.

The tumor spheroids were implanted in the window chamber directly onto the exposed dorsal skin either alone or with lung (for LLC1) or mammary (lactating female mammary fat pad for N202 and BT-474) tissue which was excised from a donor mouse and minced into small pieces in Penicillin (10,000 μg/ml—Streptomycin (10,000 μg/ml) solution. Tumors were allowed to vascularize over 7-14 days depending on model before being tested for vascular leakiness and response to therapy. Mice with BT-474 tumors were supplemented with intramuscular injection of estrogen (20 μg, twice weekly).

For the rat 13762 metastatic breast cancer model survival studies, female Fisher rats were injected intravenously via tail vein with 1×10⁴ 13762 MAT B III mammary adenocarcinoma cells. On days 20-22 after tumor cell injection, tumor-bearing rats received either a control vehicle or an intravenous radioimmunotherapy dose of 4.5 or 15 μg ¹²⁵I-mAnnA1 (specific activity=4 μCi/n). Rat body weights were monitored over time. Body weights of healthy non-tumor bearing rats were also monitored for comparison. For biodistribution studies, female Fisher rats were injected intravenously via tail vein with 1×10⁶13762 MAT B III mammary adenocarcinoma cells. Rats received an intravenous dose of 3 μg 125I mAnnA1 (20-21 μCi) at day 22 after tumor cell injection. Organs and tissues were excised, weighed, and subjected to dosimetry analysis at select timepoints after radioimmunoconjugate administration.

Tumor Growth. Tumors were imaged using intravital fluorescence microscopy, as described (Borgstrom et al., 2013; Oh et al., 2007; Oh et al., 2014). Tumor growth was analyzed off-line from the recorded, digital, grayscale 0-to-256 images using Image-Pro Plus (Media Cybernetics, Bethesda, Md.). Tumor growth was determined in 2 ways, by measuring the area with fluorescence signal from the GFP or CFP expressing tumor cells or by quantifying the cumulative fluorescence signal for the tumor over time. Tumor area is measured by counting the number of pixels with a grayscale intensity above 75, thereby making it easier to reliably follow irregularly shaped tumors. The cumulative tumor fluorescence signal was measured by signal summation of all pixels over 75. All growth curves are normalized to the tumor on day 0. In all cases, growth measured by area or aggregate fluorescence signal were found to be very similar so only one of the results is usually shown.

Fluorescent labelling of antibodies. VEGF antibody and Herceptin were fluorescently labelled using AlexaFluor 488 or 568 as per manufacturer's instruction (Invitrogen, Carlsbad, Calif.). mAnnA1 and mAPP2 antibodies were labelled as previously described (Oh et al., 2007; Oh et al., 2004; Oh et al., 2014). Size exclusion chromatography was used to separate free dye from labelled antibody (EconoPak 10 DG, Bio Rad, Hercules, Calif.).

Conjugation and radiolabelling of antibodies and peptides. Targeting antibodies (mAnnA1 or mAPP2) were conjugated to various therapeutic agents using standard methods. Briefly, doxorubicin, docetaxel, and antibody-conjugatable maytansinoids (DM1), and cisplatin were conjugated to mAnnA1 to create antibody-drug conjugates, and both drug and antibody binding activity were maintained. Cisplatin was conjugated as in (Deng et al., 2013; Shen et al., 2005). Briefly, carboxylic acid groups have been introduced onto dextran molecules by chemical modification (Shen et al., 2005). Standard acid activation chemistry is then used to attach the modified dextran to free amines on the antibody (Shen et al., 2005). The free acid groups remaining on the dextran molecule are used to chelate and carry cisplatin, the amount of which (Wakankar et al., 2010) is determined spectroscopically as in (Deng et al., 2013). We detected ˜10 cisplatin molecules per antibody. Doxorubicin was immunoconjugated and quantified as per (Griffiths et al., 2003). We conjugated antibody to drug using an acid-labile hydrazone bond to doxorubicin and a thioether bond to the antibody (mAnnA1 or control IgG) (Griffiths et al., 2003). The antibody conjugates carried ˜4 drug molecules attached site-specifically at thiols of reduced interchain disulfide bonds. Docetaxel was immunoconjugated using standard immunotoxin conjugation techniques (see Chapter 21 in Bioconjugate Techniques 2^{nd} Edition, ed. Greg T. Hermanson, 2008). DM1 was conjugated using standard linkage chemistry (see for example (Burris et al., 2011; Chari et al., 1992; Chari et al., 2014; Shao et al., 2018; Wakankar et al., 2010). Radiolabelling of antibodies with ¹²⁵I (or other radionuclides, including ¹³¹I or ¹⁷⁷Lu) was performed as described previously (D. P. McIntosh et al., 2002). Briefly, affinity purified antibodies were conjugated to ¹²⁵I using Iodogen as described (D. P. McIntosh et al., 2002) to achieve a specific activity of approximately 10 mCi/mg. For preparation of ¹⁷⁷Lu-mAnnA1, a standard 1,4,7,10-tetraazacyclododecane-1,4,7,10-tetraacetic acid (DOTA) linkage chemistry procedure was used to conjugate mAnnA1 to ¹⁷⁷Thu. VEGF antibody, Herceptin, TGF-β antibody and soluble Thy-1 were conjugated to mAnnA1 or mAPP2 using standard antibody methods yielding bi-specific conjugates (see Chapter 20 in Bioconjugate Techniques 2^{nd} Edition, ed. Greg T. Hermanson, 2008).

Conjugation and labelling of dendrimers. Prior to antibody attachment, dendrimers were modified up to 30-40% with particular moiety/chelator for coupling of each radioisotope. Remaining primary amines on dendrimer surfaces were blocked by acetic anhydride.

PAMAM dendrimers were labeled with fluorophore (AlexaFluor-488 or AlexaFluor-568) using corresponding N-hydroxysuccinimide (NHS)-activated esters reactive toward primary amines of dendrimers. Unmodified primary amines on dendrimer surface were then shielded by reaction with acetic anhydride. Fluorophore-labeled dendrimers were purified by size exclusion chromatography before antibody functionalization.

For radioiodination, the surface of PAMAM dendrimers were modified up to 30-40% with N-succinimidyl-3-(4-hydroxyphenyl)propionate. Iodine was introduced by electrophilic substitution onto aromatic ring of p-hydroxyphenyl residues. The degree of ¹²⁵I incorporation was estimated by mass spectrometry using nonradioactive “cold” iodine. Conditions were optimized for maximum iodine incorporation of ¹²⁵I. We achieved specific activities ranging from 65 μCi/μg to 2 mCi/μg (˜30 atoms of ¹²⁵I per nanoparticle).

For antibody functionalization of dendrimers, fluorophore-labeled and/or radionuclide-loaded PAMAM dendrimers were conjugated to mAnnA1 or control IgG antibodies through maleimide/thiol chemistry. Formulations with a molecular substitution ratio (MSR) of dendrimer to antibody at 1.2:1, and 4:1 were evaluated. All formulations were characterized for immunoreactivity using saturation binding assay on recombinant AnnA1 protein, where immunoreactive fraction was determined by extrapolation of binding to infinite antigen excess. The desired stoichiometry of the conjugates was confirmed by high-performance liquid chromatography (HPLC) on a size exclusion column and subjected to purification, if necessary.

Fluorescence Confocal Microscopy. Confocal microscopy was used to acquire dual fluorescence images via a Nikon E2000 microscope (20× and 60× objective lens) equipped with a Perkin Elmer UltraView SERS confocal system with an Orca ER camera. To construct movies, dual color images were taken every second; exposures for a single fluorophore were kept under 400 msec.

Tumor uptake of antibody. IVM is a powerful imaging modality that complements gamma-scintigraphy and CT-SPECT imaging, by providing greater detail to permit live, dynamic imaging of antibody binding to the EC surface as well as direct visualization of transport across the EC barrier and accumulation in the tissue interstitium and parenchyma. IVM enables real time imaging to visualize tumor penetration of different therapies. We expanded the utility of IVM by engrafting tissue fragments of various organs into dorsal skinfold window chambers enabling long term in vivo imaging (Borgstrom et al., 2013; Oh et al., 2007; Oh et al., 2014; TestaChrastinaOh et al., 2009; Valadon et al., 2006). Cellular events including the growth and connectivity of blood vessels can be viewed by both light and fluorescence microscopy. Our IVM system includes computational algorithms developed by us to assess and quantify EC surface binding and transvascular fluxes from digital movies (Oh et al., 2007; Oh et al., 2014). We first detailed our dynamic IVM imaging system in lung tissue to visualize antibody binding and EC processing in live nude mice (Oh et al., 2007).

Statistics. SigmaStat (Systat Software, San Jose, Calif.) was used to determine statistical significance. Ranked ANOVAs with the Tukey post hoc test were used and a statistical significant difference delineated if p<0.05.

Gamma scintigraphic imaging and biodistribution analysis. Monoclonal antibodies were isolated using GammaBind Plus Sepharose (Amersham, Piscataway, N.J.) and conjugated to ¹²⁵I using Iodogen as described (D. P. McIntosh et al., 2002). Biodistribution analysis was performed as described (Chrastina et al., 2010). Imaging was performed using an X-SPECT, was fitted with a parallel-hole collimator. Normal and tumor-bearing mice and rats (e.g. Her2/neu, 13762 metastatic rat mammary adenocarcinoma, TRAMPC2 transgenic model) were anaesthetized and injected via the tail vein with ¹²⁵I-labeled monoclonal antibody (1-5 μg IgG; 10 μCi/m) before being subjected to planar gamma scintigraphic imaging and SPECT-CT. After whole body imaging, in some cases, normal organs and target tissue (i.e. tumors or lung) were excised for planar imaging ex vivo.

### Example 2

### Caveolae Targeting Enhances Chemopotency at Low Doses

To assess the ability of improving drug delivery across the vascular barrier as a means to increase therapeutic potency, we conjugated several widely-used chemotherapeutics to create caveolae-targeting antibody-drug conjugates (ADC) that are very effective at low doses. We conjugated each chemotherapy (e.g. doxorubicin, docetaxel, cisplatin) to the caveolae-targeting tumor specific mAnnA1 antibody using methods and materials as described in Example 1. Each targeted drug conjugate was tested in relevant multi-drug resistant tumor models and were found to become more therapeutically effective when compared to unconjugated drug (i.e. not targeted). Results indicate that this strategy is very effective at boosting therapeutic potency at low doses. Indeed, we observed robust therapeutic responses at administered doses in the μg/kg to ng/kg range—orders of magnitude lower than maximum tolerated doses (MTD) and the very high, almost MTD levels required to see only a slow-down in tumor growth. Frequently, ultra low doses of the therapeutic moiety are enough to eradicate the tumors when administered as a targeted drug conjugate composition. Specific examples are described below.

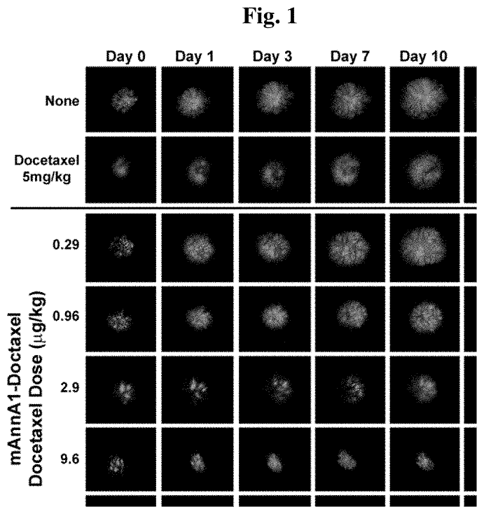

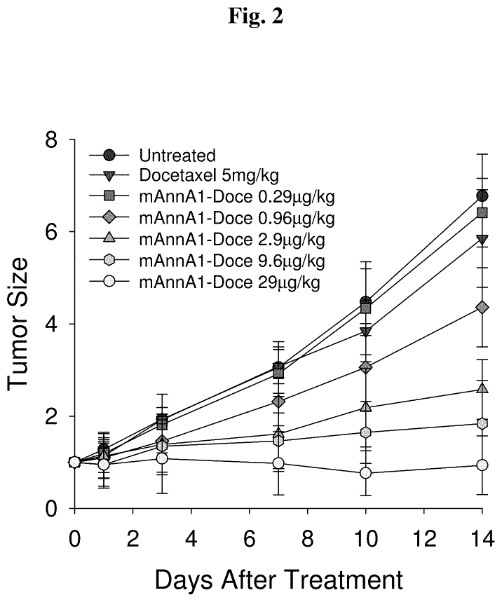

In order to estimate an effective therapeutic range for our doxorubicin ADC, we performed a 3 order-of-magnitude, dose escalation study using IVM to visualize and quantify bioefficacy in our non-permissive MDR mammary tumor model. FIG. 1 shows a dose-dependent impact with minimal benefit at 0.2 μg/kg of doxorubicin in the ADC, clear tumor regression at 2 μg/kg and eradication at 20 μg/kg. Any fluorescence signal observed after 5 days was not in intact nuclei, but as remnants of cellular debris. The control IgG-doxorubicin conjugate at 20 μg/kg of doxorubicin had no effect on tumor growth. Also, free doxorubicin at the much higher dose of 1 mg/kg had no effect at the near MTD (5 mg/kg) slowed tumor growth modestly and much less than 2 μg/kg of doxorubicin when linked to mAnnA1. Note we are comparing here the potency directly between the ADC and the drug alone based on the dose of actual doxorubicin injected. Notably, a single, ultra low dose injection of the doxorubicin conjugate killed the mammary tumor cells and caused substantial tumor regression or eradication only when conjugated to mAnnA1 (i.e. targeted drug conjugate). The data shows a clear and robust dose-dependent therapeutic response with the mAnnA1-doxorubicin conjugate. Thus, our precision delivery and therapy system derived from the caveolae pumping system and the caveolae-targeting antibody conjugate has boosted therapeutic potency of the drug by at least 2,500-fold (2 μg/kg vs. 5 mg/kg).

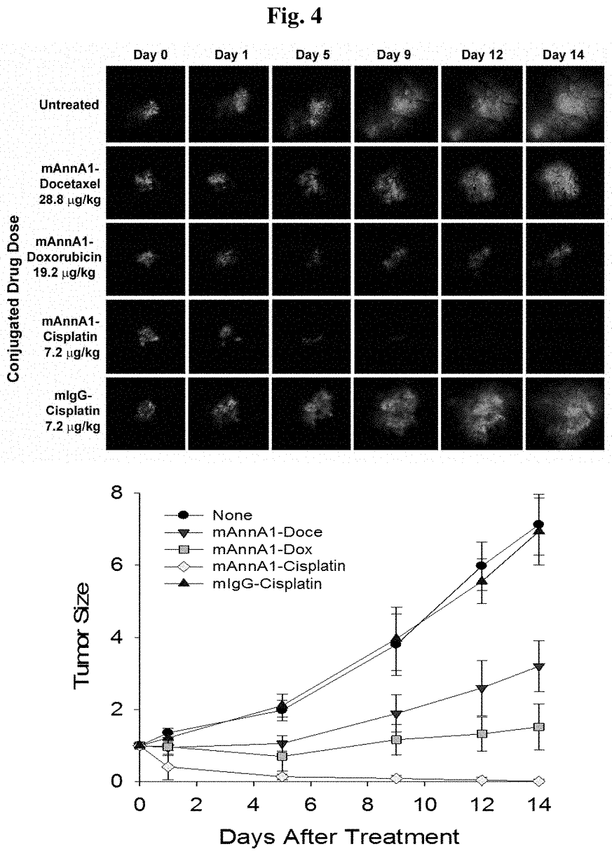

We also conjugated docetaxel directly to mAnnA1, and also observed ultra low potency of this well-known chemotherapeutic agent as a result of caveolae-pumping into solid mammary tumors. Results as shown in FIG. 4 indicate the ADC is effective at 1 μg/kg, suggesting a >1000-fold improvement in potency as compared to untargeted (i.e. unconjugated) drug that was administered at 5 mg/kg and produced less benefit. While the ADC did not completely eradicate the tumor at the highest dose tested (28.8 μg/kg), by day 14, there was a profound and significant effect on tumor size directly dependent on targeting docetaxel for improved precision transvascular delivery directly into the tumor.

Cisplatin is a widely used chemotherapy for treating solid malignancies. It has been used to treat various types of cancers, including sarcomas, some carcinomas (e g small cell lung cancer, squamous cell carcinoma of the head and neck, and ovarian cancer), lymphomas, bladder cancer, cervical cancer, germ cell tumors, prostate and breast cancer. Unfortunately, cisplatin has several serious side effects that can limit its use. We conjugated cisplatin to targeting mAnnA1 antibodies to reduce toxicity and boost therapeutic potency through improved tumor delivery and assessed the ability to eradicate tumors in IVM models.

We prepared cisplatin-carboxymethyl dextran-mAnnA1 ADC for in vivo testing. Cisplatin was conjugated as reported (SchechterPauzner et al., 1987). The free carboxylic acid groups of the dextran molecule were used to chelate cisplatin, and could also be used to conjugate other toxic metals exhibiting therapeutic activity. Using previously described methods (SchechterWilchek et al., 1987), we detected ˜10 cisplatin molecules per antibody. Affinity studies show ADC binding to its target was not impaired (data not shown). The ADC was then injected into the tail vein of mice with either prostate or mammary tumor spheroids to image and quantify therapeutic responses.

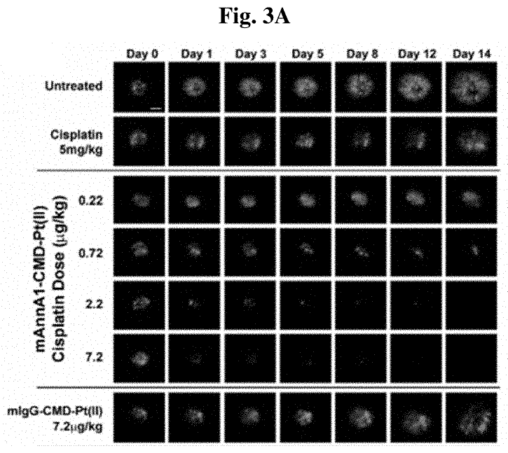

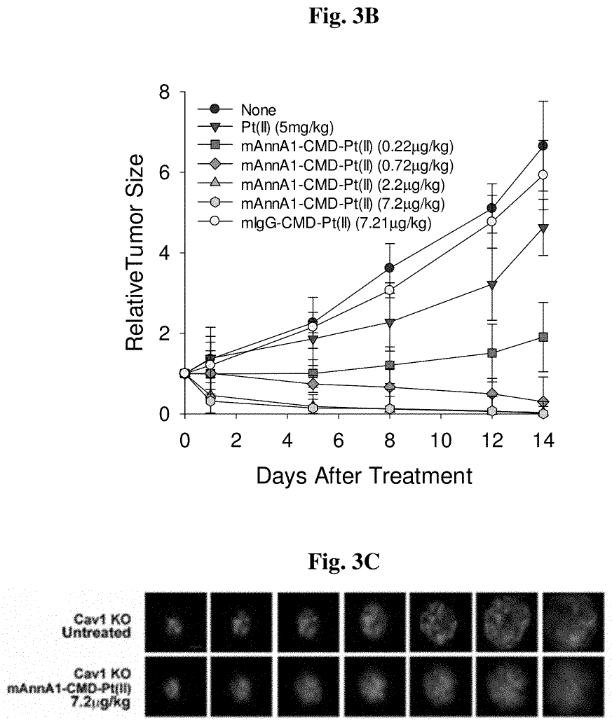

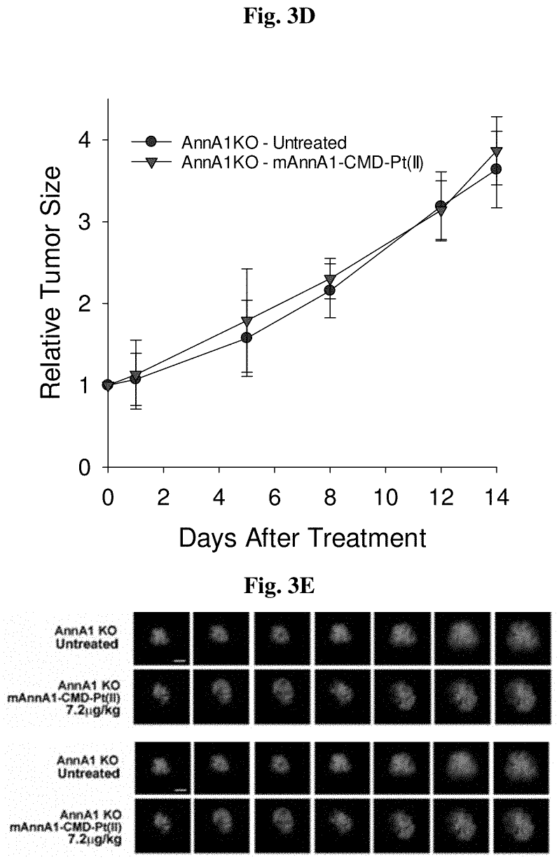

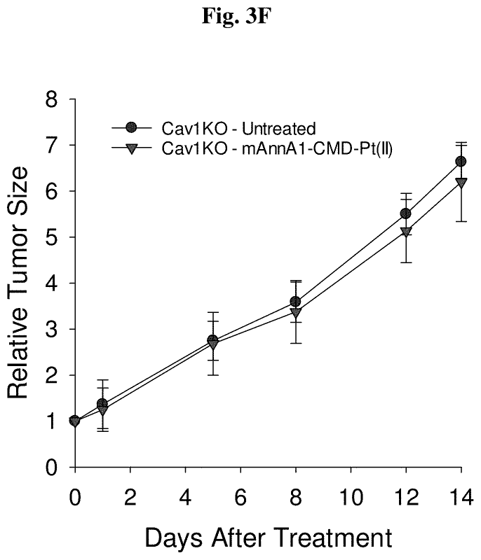

FIG. 3 shows IVM images of a dose response study of the cisplatin-CMD-mAnnA1 in a mammary tumor model system. Results show mammary tumor regression and complete destruction after a single injection at a low cisplatin dose (2.2 and 7.2 μg/kg cisplatin) in the conjugate. The control non-specific IgG-immunoconjugate at 7.2 μg/kg cisplatin had no effect. Both 10-fold lower doses (0.22 and 0.72 μg/kg) stopped tumor growth more effectively than 1000-fold greater dose of 5 mg/kg cisplatin alone. Thus, precision delivery, specifically through immunotargeting tumor EC caveolae to achieve active tumor penetration of the drug, can increase therapeutic potency >10,000-fold. Note that genetic abolition of expression of Annexin A1 and caveolin-1 eliminated therapeutic efficacy (FIGS. 3, C and E). Both the target and caveolae (the vesicular pumping mechanism) are required to get the antibody and its toxic cargo inside the tumor where the chemotherapeutic agent can reach the tumor cells and be most effective.

As shown in FIG. 4, row 4, cisplatin-mAnnA1 also eradicated TRAMP C2 prostate tumor spheroids, exhibiting a dramatic increase in therapeutic potency at low doses even when compared to docetaxel (row 2) or doxorubicin (row 3). Significant retardation of growth was observed with the mAnnA1-doxorubin conjugates (row 3) with less robust tumor eradication compared to the mAnnA1-docetaxel conjugate (row 2).

We switched to a non-IVM larger tumor burden model to assess further the efficacy of caveolae-targeted ADCs comprising mAnnA1 conjugated to the highly potent maytansinoid (DM1) toxin. We inoculated rats with 13762 metastatic rat mammary adenocarcinoma cells which colonize to the lungs after injection (Neri et al., 1982). This tumor model provides several advantages, including immunocompetency, multiple tumors, large tissue mass and tumor size, and is a model of pulmonary metastasis known to occur in breast cancer.

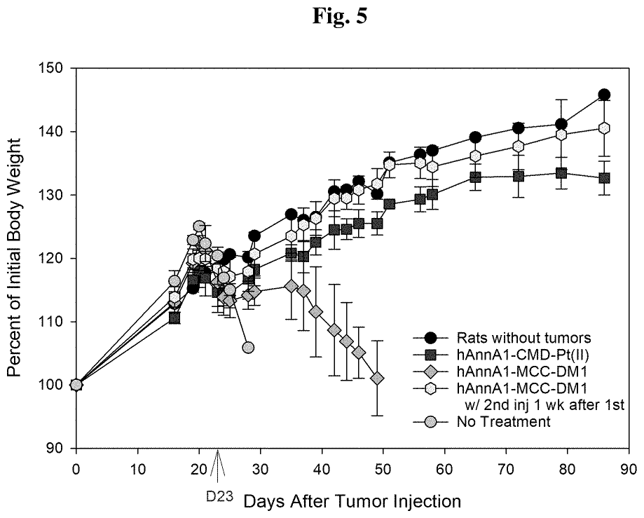

The mAnnA1-DM1 conjugate was injected on Day 21 of tumor monitoring at 15 μg per animal (equivalent to 1.5 μg/kg of DM1). The percent body weight on day of treatment for no-tumor control and tumor-bearing animals are shown in FIG. 5. The injected dose of DM1 extended survival of the animals by 25 days (FIG. 5). Two consecutive injections of the same dose on Days 23 and 30 resulted in tumor eradication and increased survival over untreated controls by >60 days, the end of the study. Note that a conjugate comprising a humanized form of the Annexin A1 targeting antibody (hAnnA1) conjugated to CMD and cisplatin (hAnnA1-CMD-Pt(II)) required one low dose to achieve an apparent cure.

To further assess the therapeutic efficacy of hAnnA1-CMD-Pt(II) in metastatic disease, the platinum containing conjugate was administered to rats injected with 13762 MAT III B adenocarcinoma cells (Neri et al., 1982). The cells (1×105) are injected via the lateral tail vein and signs of disease occur on 18-22 days post cell injection, with ample, well circumscribed, and highly vascularized tumors of 2-8 mm peppered throughout the lungs (data not shown). The rats exhibit a roughened coat and weight loss, which will be monitored for therapy initiation. The rats will be weighed daily starting on day 14 after injection of tumor cells (animals do not start showing symptoms until about day 18). The hAnnA1-CMD-Pt(II) conjugate was administered on day 20-21. Endpoint of control group typically occurs around days 30-35, within 10-15 days post-treatment.

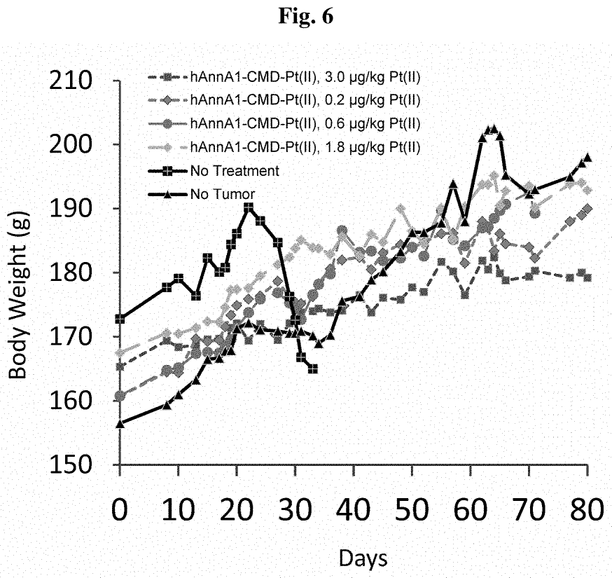

In these studies, rats with lung tumors were treated with hAnnA1-CMD-Pt(II) with Pt(II) loads varying from 0.2-3.0 μg/kg. The untreated rats died within 30-35 days, with excised lungs showing multiple tumors and hemorrhage (data not shown), while ADC-treated animals showed significant life extension to study termination (>60 days post-therapy) and an apparent lack of tumors detected in lungs. The body weight of treated animals frequently returned to normal weights of rats without tumors usually within 5 weeks (FIG. 6). Thus, in these hard to treat, resistant tumor models, the caveolae-targeting ADC produced robust tumor regression and even eradication at unprecedented low doses of drug (1.5 μg/kg cisplatin equivalents).

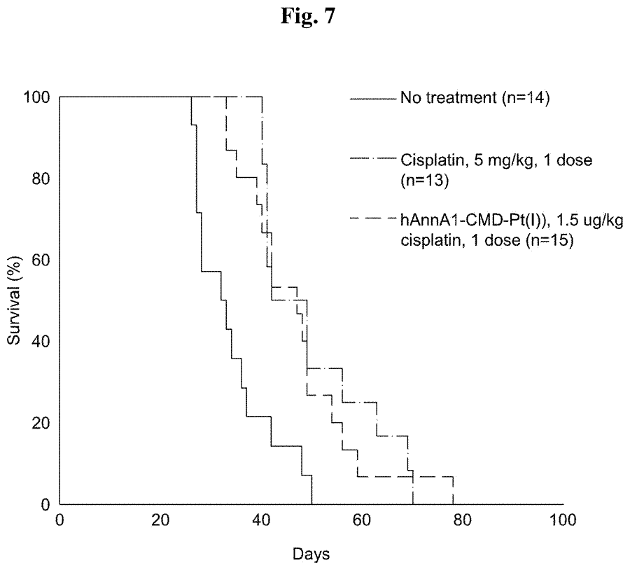

We also performed a pilot efficacy study of hAnnA1-CMD-Pt(II) in a genetically engineered mouse HER2/neu model that develops spontaneous mammary tumors (GuyWebster et al., 1992; Reilly et al., 2000). Tumor-bearing HER2/neu transgenic mice were injected with a single IV dose of either cisplatin (5 mg/kg) or hAnnA1-CMD-Pt(II) carrying 7.5 μg of conjugate, 1.5 μg/kg equivalent of cisplatin (>1000-fold less) (FIG. 7). Kaplan-Meier plots show significant mean extension in survival (30 days) with both therapies when compared to untreated controls. Importantly, no signs of toxicity were observed in mice treated with hAnnA1-CMD-Pt(II), whereas mice treated with cisplatin at 5 mg/kg always showed signs of toxicity (lethargy and body weight loss; 10 mg/kg was lethal, thus at MTD). The hAnnA1-CMD-Pt(II) immunoconjugate produced a robust therapeutic effect at ˜3,300 times lower doses (in cisplatin equivalents) than cisplatin itself.

The overall results of these studies indicate the potential utility of using the caveolae pumping system to target ultra low doses of chemotherapies to treat a variety of tumor types. Each of the above results are unprecedented; no previous delivery system has attained such results. Together, these data show that the caveolae-targeted drug immunoconjugates appear to deliver chemotherapeutics precisely into tumors, effectively to enhance therapeutic potency by 3- to 4-orders of magnitude, and in some cases, even eradicate tumors at ultra low doses.

### Example 3

### Transvacular Pumping Enhances Radioimmunotherapy

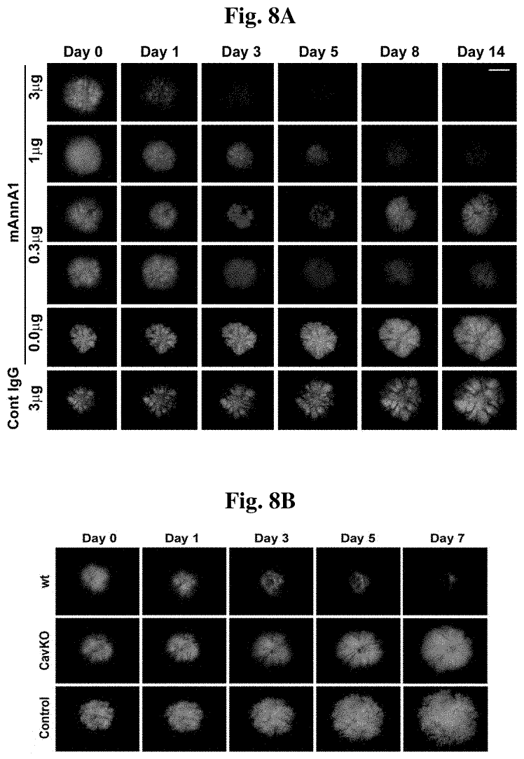

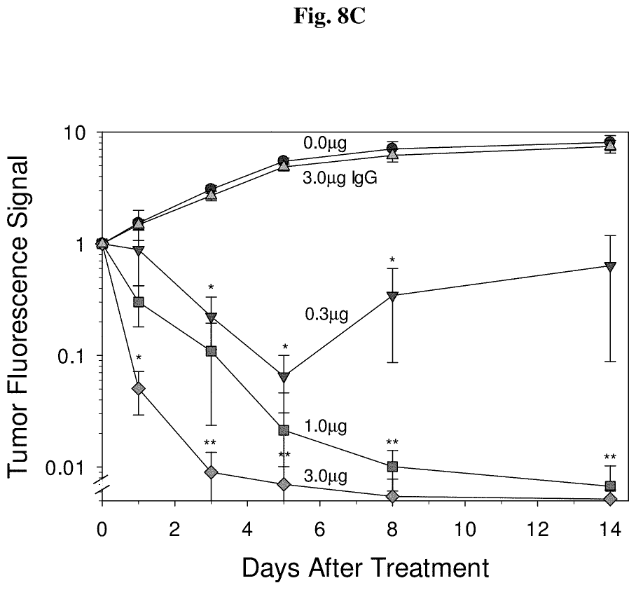

To assess the potential benefit of targeting caveolae to improve delivery and thus, enhance the therapeutic potency of radio-immunotherapy (RIT), we conjugated a well-known toxic radionuclide ¹²⁵I directly to mAnnA1 and used fluorescence IVM to visualize the effects of i.v. injected ¹²⁵I-labeled mAnnA1 (specific activity 10 mCi/mg) (FIG. 8). The highest dose (3 μg) led to obvious extensive tumor and vascular destruction within 24 hr and tumor cell eradication within 3 days. In contrast, Isotype matched control ¹²⁵I-IgG produced no effect at this dose. Contrary to our expectations, but consistent with pervasive flooding of the tumor with radioactivity delivered via targeted caveolae pumping of mAnnA1, most tumor cell damage occurred well before cessation of tumor blood flow could cause anoxia and infarction. Extensive tumor cell death and pyknotic nuclei were obvious at 12-14 hr post injection, even when ample local blood flow was still evident. Full cessation of blood flow and tumor vascular occlusion occurred at 16-30 hr. Thus, the radiation is now trapped inside the tumor where it can continue to kill until eradication is achieved. Doses as low as 0.3 μg, although less effective, did destroy tumor vessels and cells and caused significant tumor regression. Doses at or below 0.1 μg failed to slow tumor growth or damage tumor cells or vessels (data not shown). Variable tumor regrowth was apparent at 0.3 μg, but not at 1 or 3 μg where the tumors were comprehensively destroyed (FIG. 8A). A second injection into mice exhibiting regrowth produced similar dose-dependent tumor destruction. This RIT was rendered ineffective when tumors were grown in tissue from caveolin-1 knockout mice (FIG. 8B). These results demonstrate that caveolae are required to pump, to locally irradiate, and destroy tumors. Notably only a single very low μCi dose is required to cause an unexpectedly robust therapeutic response, namely complete tumor destruction.

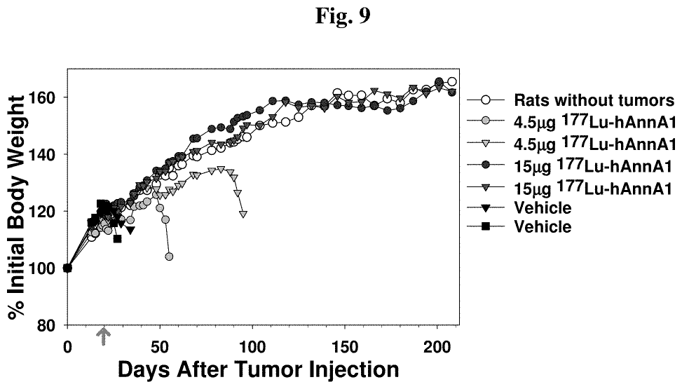

To explore the efficacies of other clinically relevant radionuclides with shorter half-lives and greater emission energies, we radiolabeled hAnnA1 with the beta emitter ¹⁷⁷Lu and intravenously injected the radioimmunoconjugate into tumor bearing rats. ¹⁷⁷Lu-hAnnA1 showed strong efficacy in the aggressive 13762 MAT IIIB rat lung tumor model where rats treated with control agents died within days of beginning therapy. The specific radioactivity of the RIC was 4 mCi/mg. Rats treated with a low dose of 15 μg ¹⁷⁷Lu-hAnnA1 survived at least 5 months after injection before the experiment ended. This was accompanied by a complete restoration of body weight to normal levels (FIG. 9). A 4.5 μg dose gave an intermediate response, extending survival beyond controls by ˜50-90 days. These micro-doses typically used for tumor imaging appears highly effective and in some cases curative for all rats treated with ¹⁷⁷Lu-hAnnA1.

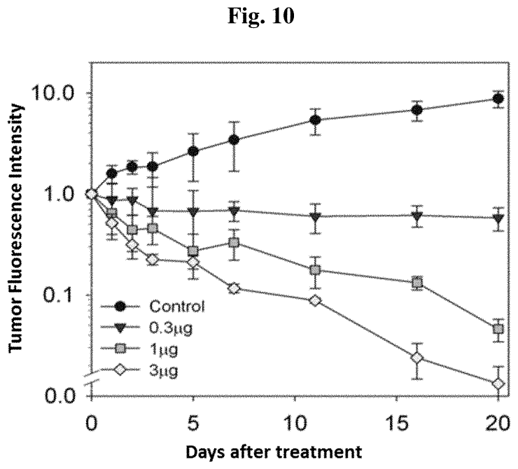

We also injected the mouse monoclonal antibody (mAnnA1) labeled with ¹⁷⁷Lu into mice bearing engrafted N202 spheroids co-implanted with mammary fat pad in window chambers as described previously. ¹⁷⁷Lu-mAnnA1 eradicated N202 mammary tumors at 3 μg in a manner similar to 125I-mAnnA1 (compare FIG. 10 to FIG. 8).

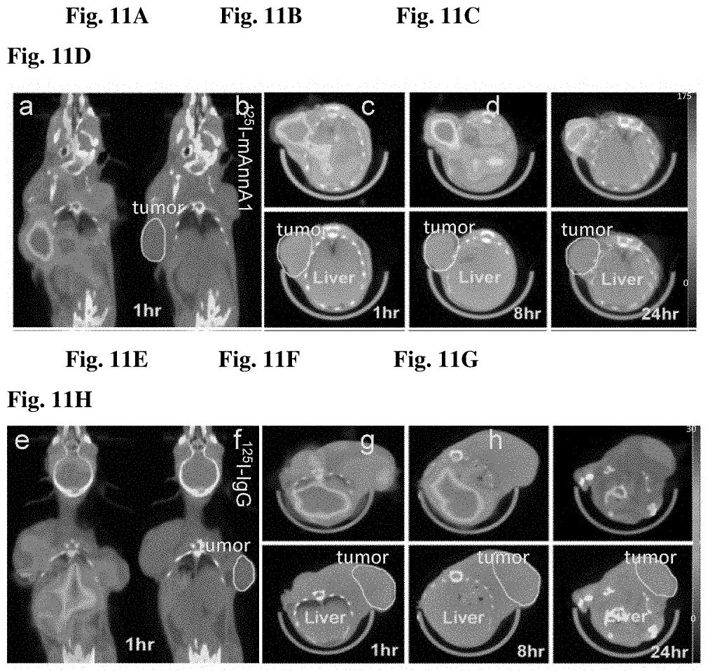

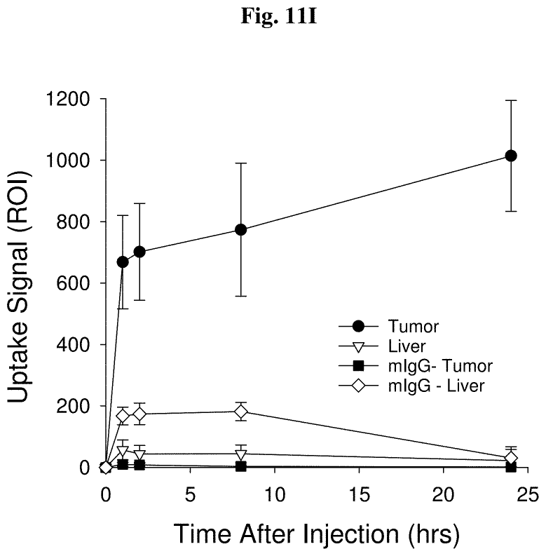

To directly assess further the radionuclide targeting capabilities of mAnnA1 in vivo, ¹²⁵I-mAnnA1 (3 μg) was injected i.v. into female Her2/Neu mice, a well-established mouse mammary tumor model (Quaglino et al., 2008). Expressing the Neu (Erbb2) gene under the transcriptional control of a mouse mammary tumor virus promoter/enhancer, these mice develop spontaneous mammary tumors (GuyCardiff et al., 1992; GuyWebster et al., 1992; Reilly et al., 2000). Tumor accumulation of ¹²⁵I-mAnnA1 produced a robust and highly localized tumor signal in SPECT/CT images captured as early as 1 h post-injection (FIG. 11). Region of interest analysis shows the tumor-specific signal persists even 24 hrs post-injection with little to no apparent accumulation in off-target organs such as the liver (FIG. 11 a-d, i), unlike as isotype matched ¹²⁵I-IgG control (FIG. 11 f-h), note the different imaging scales to enhance detection of the control IgG signal). These results clearly illustrate how caveolae-targeting improves upon passive transvascular delivery in transporting imaging agents specifically into tumors. With caveolae-targeting, we also did not detect a thyroid signal from the customary release of ¹²⁵I from dehalogenation that occurs within 6-8 hrs in blood, which is consistent with robust transvascular pumping of the radioimmunoconjugate and its rapid clearance from the blood. This data suggests caveolae pumping can achieve high intratumoral concentrations of radionuclides using low doses of antibody.

Cumulatively the data shows very specific, robust and rapid targeting of multiple solid tumors. The results from the IVM and non-IVM mammary tumor models show that caveolae pumping is useful to delivery precisely payloads across the EC barrier, effectively concentrating them in the tumor interstitium and parenchyma where a tumor cell killing agent could be most effective. It does so in small drug-resistant IVM tumors, spontaneous tumors in immunocompetent transgenic mice, and also in large tumor burden hard-to-treat metastatic models of disease.

These data indicate that the 34 kD truncated form of AnnA1 is specifically exposed to the blood only on tumor endothelium and readily accessible to i.v. injected antibodies. The Annexin A1 protein is known to be expressed intracellulary in other cell types (e.g. neuronal, endocrine, some leukocyotes, but not normal endothelium (Gerke and Moss, 2002)). Our studies with radiolabeled mAnnA1 in vivo clearly show that the 34 kD truncated AnnA1 recognized by our targeting antibodies (mAnnA1 and hAnnA1) is readily accessible in the tumor vasculature with little to no antibody binding to other cell types (Dun et al., 2004; Oh et al., 2004; Oh et al., 2014).

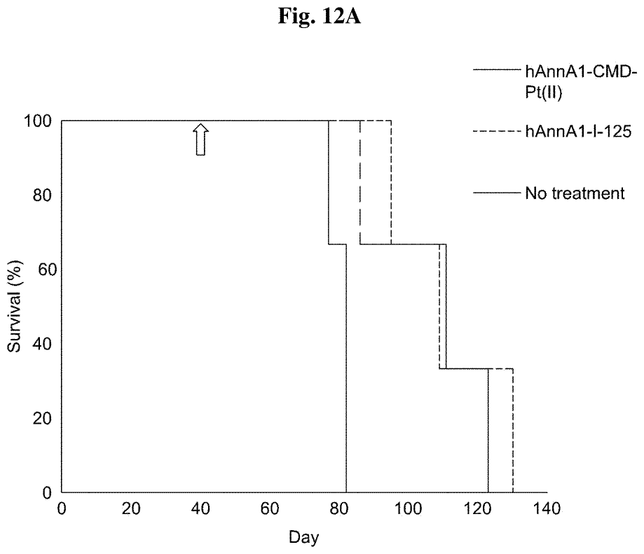

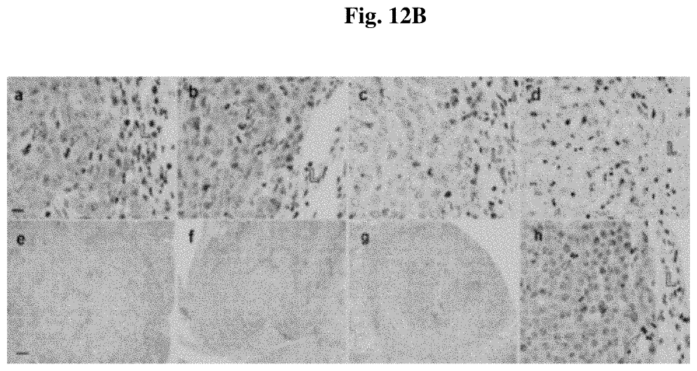

In order to test the efficacy and potency of hAnnA1 conjugated to either CMD-Pt(II) or ¹²⁵I in metastatic tumors of human origin, we established the NCI H209 tumor model in Rag2/IL2RG (R2G2) mice Animals developed sizable tumors in 40 days. Injection on the specified days (starting from the Day 41, see FIG. 12A) of either hAnnA1-CMD-Pt(II) at ultra-low dose 1.5 μg/kg equivalent of cisplatin, or 8 μg of radioimmunoconjugate ¹²⁵I hAnnA1 (specific activity of 6.5-7.4 μCi/kg) extended survival of animals by ˜50 days with no observable side effects (FIG. 12A).

Macroscopic examination from another study conducted in the 13762 rat metastatic breast cancer model using ¹²⁵I-mAnnA1 as a radioimmunotherapy revealed no tumors in lungs or elsewhere (data not shown). Tissue histology showed that the caveolae-targeted radioimmunotherapy destroyed metastatic lumg tumors (FIG. 12B). At day 1 (subpanel a) day after i.v. injection of ¹²⁵I-mAnnA1, little effect was apparent with ample tumor cell division still evident. But by day 3 (subpanel b) and 5 (subpanel c), tumor cell death was rampant. By day 7 (subpanels d-g), there was a pervasive loss of normal histological features and extensive chromatin condensation throughout the tumor. Examination at low magnification revealed destruction throughout tumors of all sizes. The control ¹²⁵I-IgG was not effective (subpanel h). Together, these results illustrate the success and feasibility of enhancing the potency of therapeutic agents at ultra low doses by directly targeting them for transvascular transport to overcome the vascular barrier and improve precision drug delivery directly into diseased tissue.

### Example 4

### Enhancing the Potency of Biologics: Antibodies and Peptides

Having demonstrated the feasibility of enhancing the therapeutic activity of RIT and conventional chemotherapies at ultra low doses, we explored enhancing the potency of various biological therapies, including therapeutic antibodies and peptides.

We assessed tumor antigen targeting and the efficacy of therapeutic antibodies conjugated directly to a caveolae targeting antibody in an IVM EO human breast cancer model. We co-implanted human breast donor tissue with human breast tumor spheroids into the dorsal skin of nude mice fitted with window chambers for IVM imaging, using methods similar as previously described (Borgstrom et al., 2013). The human blood vessels in these implanted tissue fragments readily connect with the host mouse vasculature, permitting blood flow throughout the implant and retaining their species-specific markers (human Ulex europaeus I and mouse-specific PECAM; data not shown). This ‘human-on-human’ approach offers the ability to validate accessible human EC markers and targeted probes in an in vivo IVM model with functional human blood vessels.

To assess the feasibility of enhancing the low dose potency of therapeutic antibodies using a bifunctional antibody approach, we conjugated mAnnA1 directly to antibodies against vascular endothelial growth factor (VEGF). Anti-VEGF antibodies suppresses the growth of new blood vessels by inhibiting cell signaling pathways that promote angiogenesis. Anti-angiogenic drugs, here exemplified by anti-VEGF antibodies, neutralize the function of VEGF primarily expressed by tumor and immune (macrophage) cells inside the tumors, and importantly, are located within the tumor interstitium and extracellular matrix surrounding all cells within the tumor. Notably, this bifunctional antibody is comprised of a precision delivery moiety (AnnA1 antibody) and a therapeutic moiety (anti-VEGF antibody).

The humanized form of the anti-VEGF monoclonal antibody is known as bevacizumab (marketed as Avastin). Bevacizumab was used as a first-line of therapy for metastatic colorectal cancer, thus validating the idea that VEGF is a key mediator of tumor angiogenesis and that blocking the formation of new blood vessels is an effective strategy to treat solid tumors. It is now considered a conventional chemotherapy to treat different types of cancers (brain, kidney, lung, cervix, ovary or fallopian tube), but has also been used to treat other indications, including diabetic retinopathy and age-related macular degeneration. Administration of bevacizumab frequently leads to adverse side effects because of systemic exposure. Main effects include hypertension and a heightened risk of bleeding. Bowel perforation has been reported, and fatigue and infection are also common. These adverse events are largely avoided in opthalmological use since the drug is introduced directly into the eye, thus directly accessing its target and minimizing any effects on the rest of the body.

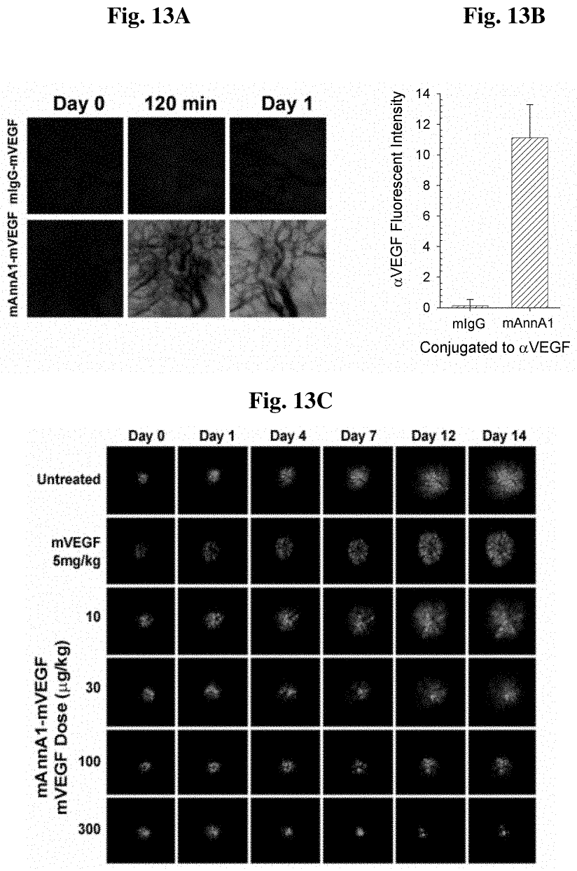

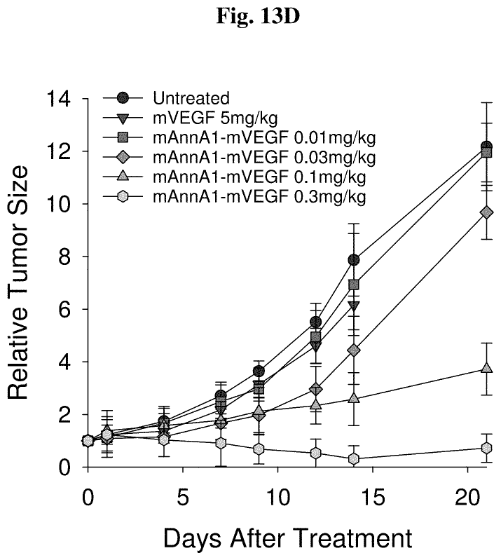

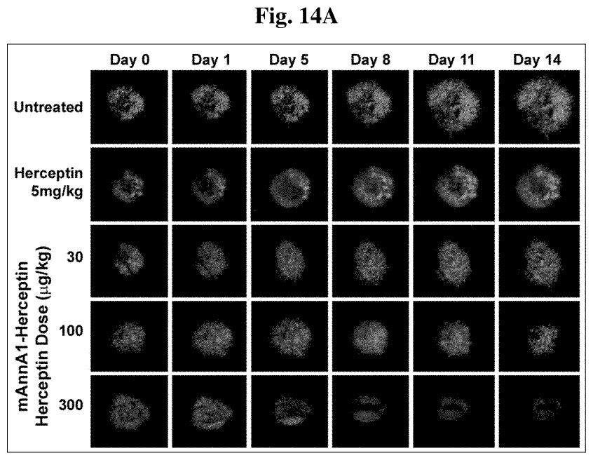

To improve the precision delivery of this anti-angiogenic agent and achieve therapeutic efficacy at ultra low doses, we conjugated VEGF antibodies (mVEGF) directly to mAnnA1. We compared mAnnA1-mVEGF antibody conjugates and mVEGF alone in an IVM EO mammary tumor model with spheroids of H2B-GFP expressing mammary tumor cells (N202) and co-implanted orthotopic tissue from normal mice. We assessed efficacy, transvascular flux and overall tumor uptake (FIGS. 13A and B). We measured the uptake of mAnnA1-mVEGF as compared to non-specific IgG-mVEGF control dual-antibody conjugate. Antibody conjugates were labeled with GFP and injected into the tail vein of mice that were then imaged over the course of hours and days to assess accumulation of fluorescent signal within the tumor (FIG. 13A). Results indicate 100-fold more mVEGF uptake by the tumor (based on fluorescent intensity) when conjugated to mAnnA1 as opposed to the non-specific IgG isotype matched control (Fib. 13B).

We then assessed therapeutic activity and followed tumor growth over 14 days. FIG. 13C shows that the mAnnA1-mVEGF conjugate had little effect on halting tumor growth at the equivalent of 10 μg/kg mVEGF, but significantly retarded growth at 30 μg/kg compared to 5 mg/kg of mVEGF alone. Indeed, tumor growth was not affected with administration of mVEGF alone, even at this high dosage, when compared to the untreated tumor control (top 2 rows). In contrast, comparison of tumor size from Day 0 to 14 indicates that administration of 0.1 or 0.3 mg/kg of mAnnA1-mVEGF essentially stops tumor growth (bottom 2 rows; FIG. 13C). The boost in potency of a therapeutic antibody with anti-angiogenic activity was significant and unexpected.

To extend our findings to another therapeutic antibody commonly used to treat solid cancers, we conjugated mAnnA1 directly to traszutumab (marketed as Herceptin). Herceptin binds to HER2 growth factor receptors on the surface of breast cancer cells effectively inhibiting their functions, including cell signaling, ultimately, causing cancer regression. Herceptin is frequently used in the clinic and considered a flagship antibody of a modern class of targeted biologics. Targeted cancer therapies designed specifically against tumor cell surface receptors, biomarkers, and functionally important surface antigens are exemplified by traszutumab, which functions through specific recognition and inhibition of the critical tumor cell surface growth factor receptor Her2/neu (also called ErbB2) which turns off one or more key signaling cascades (e.g. AKT, map kinase) that promote over-proliferation through over-expression of the HER2 growth factor receptor on tumor cells. Traszutumab, when bound at the tumor cell surface, induces immune cells to kill that cell, and thereby, initiates inherently antibody-dependent cell-mediated cytotoxicity. It also induces and suppresses specific gene expression that has affects outside the tumor cell and inhibits tumor growth by several mechanisms, including inhibiting angiogenesis.

The positive outcomes in patients treated with Herceptin belies the reality that very little drug penetrates vascular barriers and accumulates inside tumors. Better intratumoral access through caveolae pumping may allow more antibody to reach tumor cell surface receptors (i.e. Her2) and inhibit tumor growth. Therapeutic concentrations within the tumor may be attainable with caveolae-mediated precision delivery, hopefully at low doses. We therefore, tested whether precision delivery via caveolae targeting of traszutumab improves the efficacy of a therapeutic antibody that specifically recognizes an antigen expressed on the surface of tumor cells at doses significantly lower than what is typically administered. Notably, the dual antibody conjugate we generated (mAnnA1-Her2) is comprised of a therapeutic moiety and a precision delivery moiety, both of which bind tumor-specific antigens, albeit at different locations—inherently accessible EC surfaces (AnnA1) versus actual breast cancer cells (Her2 receptors).

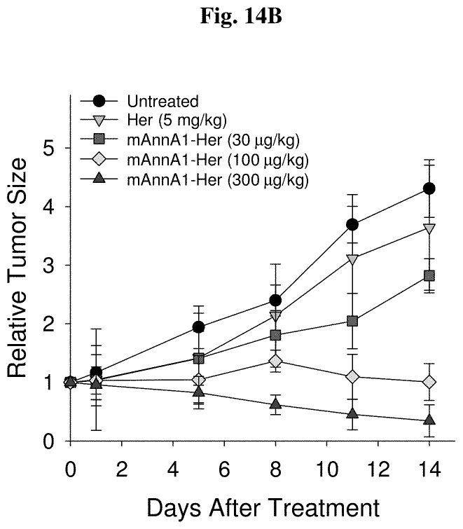

We compared mAnnA1-Herceptin conjugates and Herceptin alone in an IVM model with spheroids of GFP-tagged human BT474 (HER2 positive) cells implanted into human donor mammary tissue. FIG. 14 shows that the mAnnA1-Herceptin conjugate had little effect at 30 μg/kg equivalent dose of Herceptin, stopped BT474 tumor growth at 100 μg/kg, and induced substantial regression at 300 μg/kg. Herceptin alone at 5 mg/kg slowed growth, but much less than 100 μg/kg of the dual conjugate (mAnnA1-Herceptin). The boost in Herceptin potency was dramatic and surprising (>100-fold as per molar equivalence).

The above studies represent a direct side-by-side comparison of tumor uptake and efficacy resulting from two very different targeting strategies; namely, targeting the caveolae pumping system to improve precision delivery to enhance therapeutic potency versus targeting a cell surface tumor antigen. It thus appears that caveolae-targeting can enhance the therapeutic potency of other antibodies as a direct consequence of improved delivery that concentrates low doses inside tumors where it can be most effective. Moreover, the mAnnA1-mVEGF and mAnnA1-Herceptin conjugates represent a unique class of bifunctional antibodies optimized and targeted for transvascular transport to enhance the potency of low doses of the therapeutic antibody as a result of achieving precision delivery.

We extended our caveolae-targeting strategy to enhance the therapeutic efficacy of pharmaceutically active biologics at low doses to treat diseases affecting the lungs and pulmonary function. Previous work had identified the ability of antibodies against aminopeptidase 2 (APP2) to target lung vascular EC caveolae and facilitate rapid uptake of attached cargo (Chrastina et al., 2011; Homan et al., 2010; McIntosh et al., 2000; Oh et al., 2007; Valadon et al., 2010). Using several advanced imaging modalities, including whole body live animal imaging, we demonstrated rapid, specific and active binding and transport of labeled monoclonal APP2 antibodies (mAPP2) to EC in the lung. Pumping of mAPP2 was caveolae-dependent and did not occur in caveolin-1 knockout lung tissue that lacked functioning caveolae. Region-of-interest analysis of the imaged uptake in the lung over time (1 g wet weight), and biodistribution analysis showed robust and sustained mAPP2 accumulation in the lung (average of ˜80% of injected dose per gram of tissue (ID/g) within 30 min of injection); signal in the lung persisted for at least 2 days (Oh et al., 2007; FIG. 6m). Minimal uptake other organs (<1% ID/g) and rapid blood clearance of the labeled antibody all support very rapid and specific lung tissue uptake of caveolae-targeted mAPP2 within the first minute after injection.

We attached APP2 targeting antibodies (mAPP2) directly to target mediators with known roles in fibrogenesis and wound healing, such as occurs during acute lung injury (ALI) and the development of idiopathic pulmonary fibrosis. A staggering number of biological molecules have been implicated as therapeutic targets for pulmonary fibrosis. As the principal paradigm of our targeted delivery strategy is to enhance therapeutic efficacy at ultra low doses through tissue-specific transvascular transport, we selected transforming growth factor-beta (TGF-β), a therapeutic target with a well-known role in fibrogenesis and for which blocking antibodies have demonstrated modest anti-fibrotic efficacy (Ask et al., 2006; Brown K K, 2008; Denton et al., 2007; Khaw et al., 2007; Kim et al., 2005; Li et al., 2011; Siriwardena et al., 2002).

TGF-β is a multifunctional cytokine that has been implicated as a ‘master switch’ in induction of fibrosis in many organs including the lung (Sime and O'Reilly, 2001). The TGF-β1 isoform is thought to play the most significant role in wound healing and subsequent fibrosis (Flanders, 2004). TGF-β and TGF-β-responsive genes are upregulated in lungs of patients with IPF (Kaminski et al., 2000; Konigshoff et al., 2009; Lazenby et al., 1990; Tager et al., 2004; Vyalov et al., 1993; Zhang et al., 1996). Overexpression of active TGF-β 1 in rat lung induces a dramatic fibrotic response (Sime et al., 1997), whereas inhibiting TGF-β signaling pathways can prevent bleomycin-induced pulmonary fibrosis (Anscher et al., 2006; Ask et al., 2006; du Bois, 2010; Giri et al., 1993; Kim et al., 2005; Pittet et al., 2001; Wilson et al., 2010; Zhang et al., 1995). Importantly, inhibition of TGF-β signaling by deletion of TGF-β receptor type II specifically in the epithelium ameliorates lung fibrosis, supporting a role for epithelial TGF-β signaling in fibrogenesis (Li et al., 2011). TGF-β is chemotactic for fibroblasts and myofibroblasts (Postlethwaite et al., 1987), stimulates differentiation of fibroblasts into myofibroblasts while suppressing myofibroblast apoptosis (Zhang and Phan, 1999), stimulates production of collagen (Raghu et al., 1989) and other ECM proteins by fibroblasts (Ignotz and Massague, 1986), and inhibits matrix degradation (Edwards et al., 1987). TGF-β neutralizing antibodies (mTGF-β) have entered clinical trials for treatment of surgical scarring, sclerosis, and IPF (Ask et al., 2006; Denton et al., 2007; Khaw et al., 2007; Siriwardena et al., 2002).

Although TGF-β is clearly an excellent therapeutic candidate, inhibiting its function ubiquitously in the body present many problems. TGF-β is ubiquitously expressed, plays important roles in immune function, wound and cartilage repair (Blaney Davidson et al., 2007) and blood vessel stability (Sounni et al.), and can act as both a tumor suppressor and a tumor promoter (Yang and Moses, 2008). Indeed, knockout of any of the TGF-β genes is perinatally lethal due to severe inflammatory responses (Kulkarni et al., 1993) and a wide range of defects in normal organ development (Kaartinen et al., 1995; Sanford et al., 1997) and vasculogenesis (Martin et al., 1995). Though manipulating levels of TGF-β may have significant therapeutic value, there is much concern about the danger of off-target toxic effects (Blaney Davidson et al., 2007; Maher et al., 2007; Prud'homme, 2007). Precision delivery of TGF-β inhibitors inside lungs could be a powerful new therapeutic approach because higher concentrations inside lungs could be achieved and broad effects on normal physiological functions of TGF-β in other parts of the body would be limited.

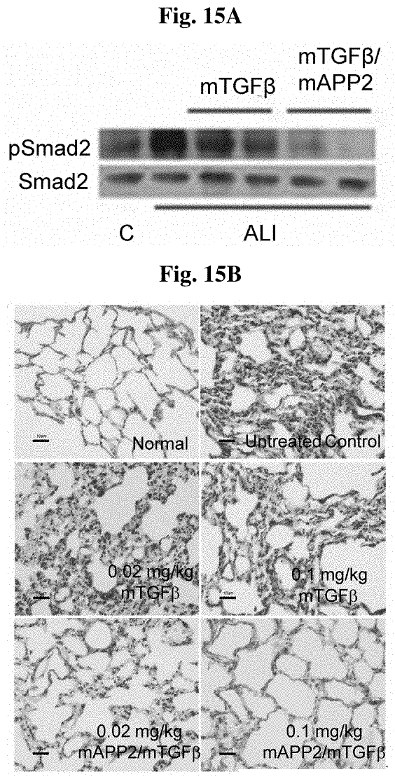

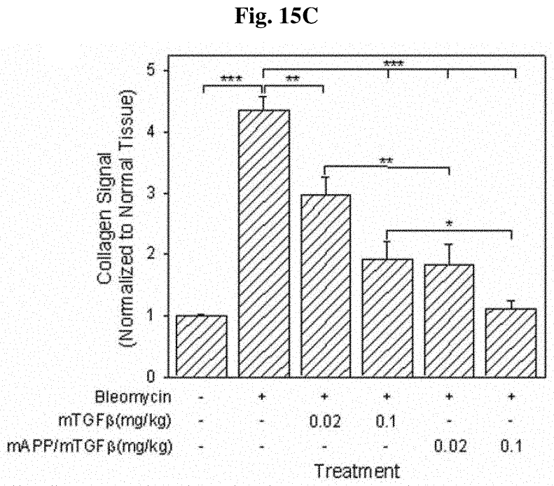

We found in agreement with published studies (Babin et al., 2011; Shaker and Sourour, 2011) that rats administered intratracheal bleomycin develop pulmonary fibrosis (histopathology, increased myofibroblasts and collagen, SMAD activation) within 8 days. First, to see if our precision delivery to the inside of the lungs could prevent TGF-β-dependent signaling and fibrosis in lung in vivo, we injected rats with mAPP2-mTGF-β bifunctional antibody conjugates, and assayed for pSMAD2 activation 6 hr following induced acute lung injury (i.e., simultaneous administration of antibody ±mAPP2 and bleomycin). Targeted delivery of mAPP2-mTGF-β prevented activation of pSMAD2 (FIG. 15A), a key mediator of TGF-β signaling. The anti-fibrotic effect of this targeted bifunctional immunoconjugate was also evident as shown through IHC analysis (FIG. 15B) and measurement of lung collagen content (FIG. 15C). Comparison of collagen deposition using Trichrome staining (FIG. 15B) and morphometric analysis (collagen signal; FIG. 15C) revealed that the response to treatment was improved for the caveolae-targeted mAPP2-mTGF-β immunoconjugate than to mTGF-β alone. Indeed, we found that even at 10-fold lower doses of mTGF-β on a molar basis inhibited fibrosis when the therapeutic antibody as conjugated to the mAPP2 targeting antibody. Notably, mAPP2 alone had no effect (data not shown). These results show that a caveolae-targeted bifunctional immunoconjugate (mAPP2-mTGF-β) can be delivered effectively to the lung in a functional form to block TGF-β signaling and fibrosis, even when administered at as a single prophylactic dose, and is superior to untargeted mTGF-β that lacks the ability to be pumped across the pulmonary vascular EC barrier. This results in a bolus delivery of therapeutic agent into the lung to achieve rapidly high local concentrations. Otherwise, the mTGF-β passively enters the lungs and many other organs, thereby achieving neither fast nor complete delivery into the lungs.

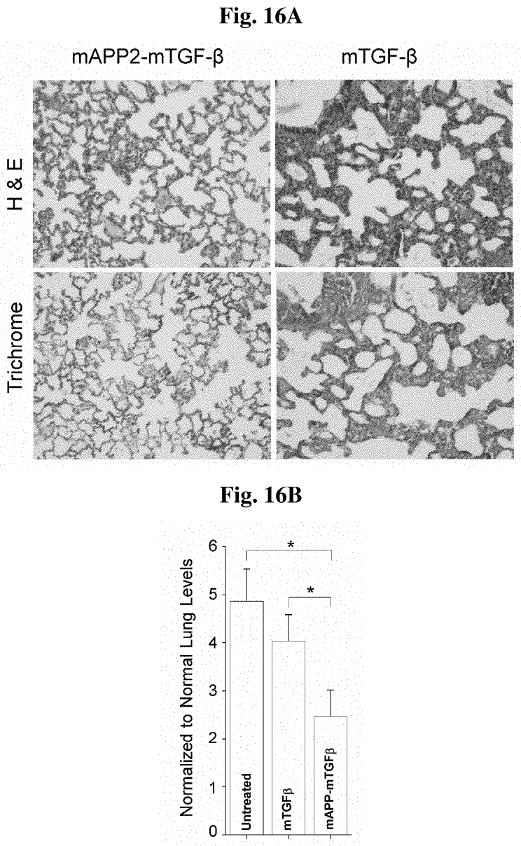

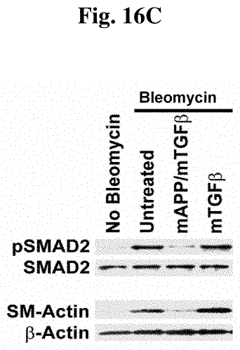

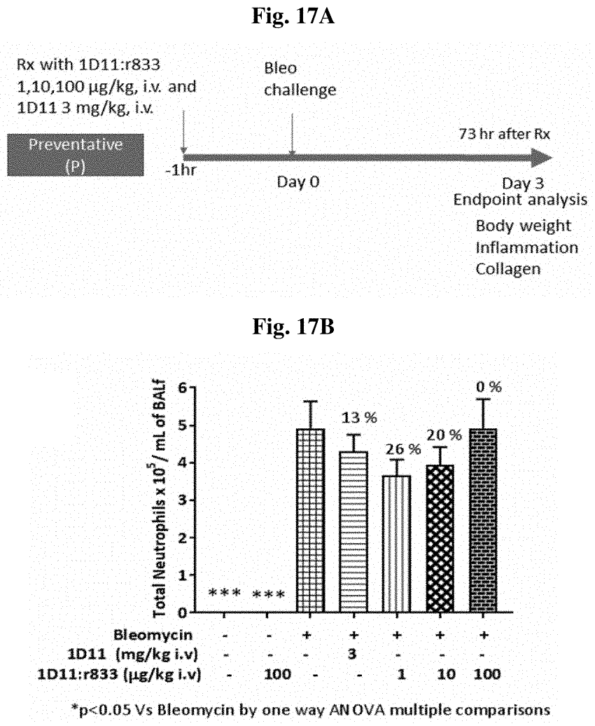

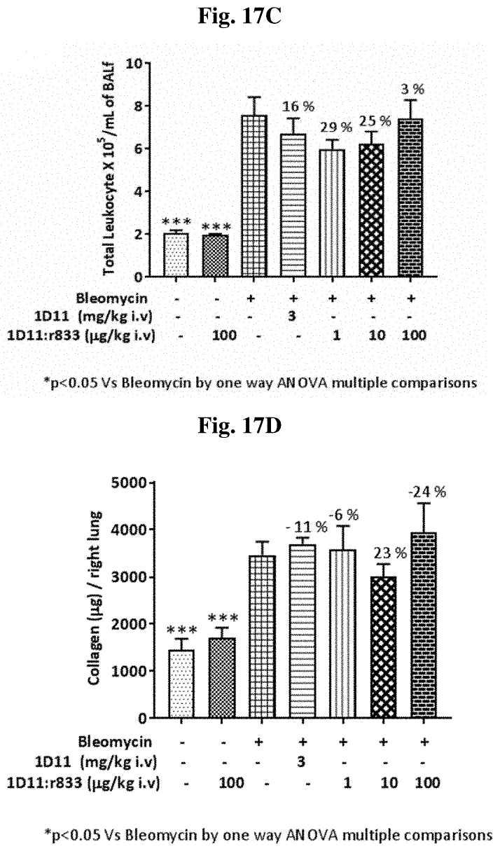

The data described above indicate that caveolae/APP2-targeted lung delivery of mTGF-β significantly enhances its anti-fibrotic effects to prevent bleomycin-induced fibrosis and collagen accumulation in models of acute lung injury (FIG. 16). To test efficacy under conditions that are clinically most relevant for patient therapy, we induced pulmonary fibrosis using IT bleomycin and waited 12 days when fibrosis was readily evident and then injected i.v. mTGF-β alone or conjugated with mAPP2. One week later after only one dose, lungs show clear enhancement in therapeutic efficacy with preservation of epithelial integrity, little optimization and no combinations, all of which should further enhance bioefficacy. Both H&E and trichrome staining (FIG. 16A) show the lungs are much more normal when mTGF-β is linked to mAPP2. Sircol assays confirm that lung collagen levels are decreased by ˜50% (FIG. 16B). Western analysis also shows that mAPP2-mTGF-β succeeded at a remarkably low dose for an antibody therapeutic (0.1 mg/kg) to reduce myofibroblast biomarker expression (αSMA) almost to normal levels and to turn off key TGF-β signaling via SMAD (FIG. 16C). Despite a 2-fold greater dose on a molar basis, the unconjugated mTGF-β did not reduce myofibroblast proliferation or fibrotic signaling. As expected, mAPP2 alone neither inhibited fibrosis nor fibrotic signaling (data not shown).

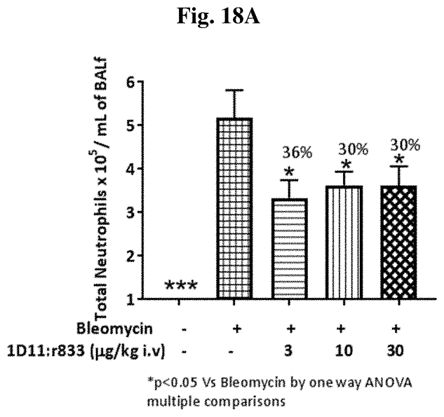

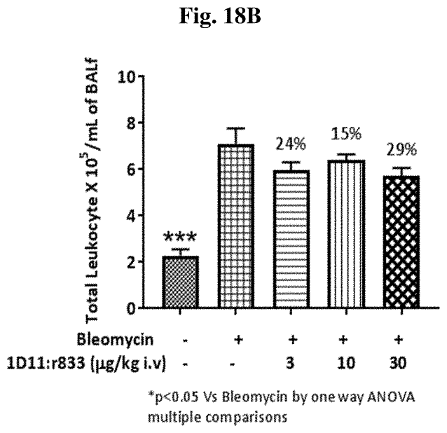

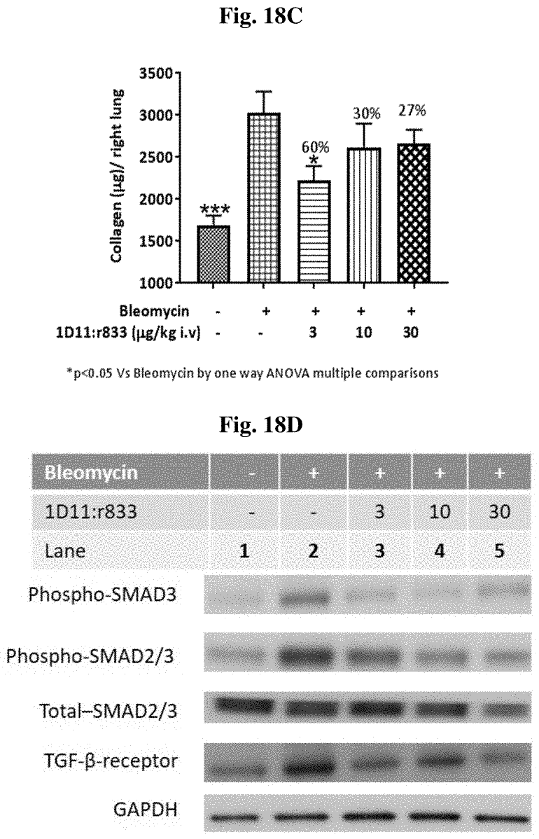

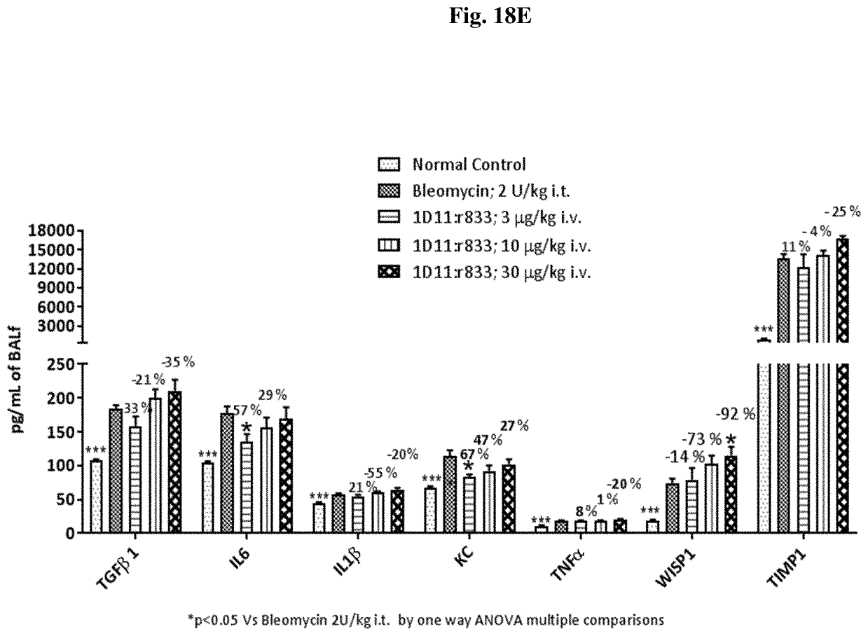

To further assess the efficacy of the bispecific antibody conjugate, we examined the effect of a single injection of the immunoconjugate comprised of the lung targeting mAPP2 (r833) and mTGF-β (1D11), referred to as 1D11:r833. We used a 3-day rat bleoymin model to induce a fibrotic pulmonary response using i.v. injection to administer the therapeutic (FIG. 18A). Briefly, female Sprague-Dawley rats were divided into 7 experimental groups: untreated controls (n=2), 100 μg/kg 1D11:r833 with no bleomycin treatment (n=2), bleomycin treatment only (n=4), bleomycin followed by 3 mg/kg 1D11 (n=5), and bleomycin followed by 1, 10 or 100 μg/kg 1D11:r833 (n=4 each). The animals were challenged intratracheally with 2 U/kg bleomycin or PBS. The respective groups were treated i.v. with 1D11 or 1D11:r833 or PBS 1 hr prior to bleomycin or PBS challenge (FIG. 18A). Animals were monitored for health and bodyweight. Upon sacrifice, bronchoalveolar lavage fluid (BALF) and lungs were harvested for further analysis.

The BALF samples were analyzed for inflammation (FIGS. 18B and C). The rats challenged with bleomycin showed an increase in total leukocyte count compared to normal control rats, indicating increased inflammation. Further, smear samples were analyzed for differential cell count and results showed that a majority of the cells were neutrophils in bleomycin challenged rats compared to normal control animals (FIG. 18B). Single injection of 3 mg/kg 1D11 did not inhibit total leukocytes and neutrophils (FIGS. 18B and C). No difference in differential cell count was observed for normal control animals or those dosed with 100 μg/kg 1D11:r833. However, single injection of 1D11:r833 at 1 and 10 μg/kg showed 25-30% inhibition of total leukocytes and 20-26% inhibition of neutrophils. (FIGS. 18B and C).

The lungs were homogenized and analyzed for lung collagen content by Sircol assay. In bleomycin control animals, there was a significant difference in collagen content observed compared to control animals (FIG. 18D). Rats treated 1 hr. before bleomycin challenge with 1, 10 and 100 μg/kg 1D11:r833 showed −6%, 23% and −24%, respectively, of inhibition of lung collagen content. 1D11 alone did not inhibit collagen deposition in the lung. The data suggest apparent inhibition of inflammation and collagen deposition at 1 and 10 μg/kg of 1D11:r833, but not at the higher 100 μg/kg dose. Based on these results, we next performed a study to evaluate the effect if single injections of the bispecific antibody conjugate at doses between 3 and 30 μg/kg.

The experimental design was essentially the same as described above, with n=3 for the untreated control group and n=5 for all others, including bleomycin only treated animals One hour prior to intratracheal challenge with 2 U/kg bleomycin or PBS (controls), animals were treated i.v. with 3, 10 or 30 μg/kg 1D11:r833 Animals were monitored for health and bodyweight for 3 days and then sacrificed prior to harvesting of BALF samples and lungs. The efficacy of 1D11:r833 was evaluated for endpoints that included body weight loss, pulmonary inflammation, and biomarker analysis from BALF and lung homogenate samples.

Bleomycin challenge induces significant body weight loss in animals and we found that 1D11Lr833 did not product animals from weight loss compared to controls over the 3-day period (data not shown). BALF collected on day 3 was analyzed for inflammatory readout (i.e. total and differential leukocyte count) (FIGS. 18 A and B). Bleomycin induced significant increases in total leukocytes (B) and in differential cell counts, with a significant increase seen in total neutrophils (A); 15-30% inhibition of leukocytes at all 3 doses of 1D11:r833 and 30-36% inhibition of neutrophils).

Lung homogenates were analyzed for collagen content using the Sircol method. Rats pre-treated with 3 μg/kg 1D11:r833 prior to bleomycin challenge showed significant reduction in collagen content in response to bleomycin (reduced by 60% compared to control animals), whereas doses of 10 and 30 μg/kg 1D11:r833 showed reductions of 30% and 27%, respectively, compared to controls (FIG. 18C). Homogenate samples were also analyzed for TGF-β downstream signaling by Western blot probed with primary antibodies against phospho-SMAD3, phospho-SMAD2/3, TGF-β receptor and GAPDH (control). Bleomycin treatment showed up-regulation of phosphorylation of SMAD3 and SMAD2/3, with increased expression of TGF-β receptor (FIG. 18D). Treatment with 1D11:r833 showed inhibition of downstream signaling, with a reduction in phosphorylation of SMAD and TGF-β receptor expression. Next, we analyzed the effect of 1D11:r833 on BALF cytokines: TGF-β1, IL-6, IL-113, KC/CXCL1, TNF-α, WISP1 and TIMP1. Results indicate non-significant inhibition by 1D11:r833 of TGF-β1 and IL-10 as compared to controls (FIG. 18E). TNF-α, WISP1 and TIMP1 were not affected at the 3 μg/kg 1D11:r833 dose, whereas treatment with 10 and 30 μg/kg 1D11:r833 showed a dose-dependent upregulation of these biomarkers (FIG. 18E right-side columns).

Together these results suggest efficacy of a TGF-β neutralizing antibody can be achieved at an ultra low dose (e.g. 3 μg/kg) when conjugated to an antibody that specifically targets lung vascular endothelia cell caveolae. Therefore, pumping into lung via mAPP2 renders the mTGF-β therapeutic antibody much more effective not only prophylactically in halting signaling and disease progression but also therapeutically in halting and possibly reversing established fibrosis.

### Example 6

### Nanoparticles and Other Carrier Systems

Nanotechnology has the potential to offer paradigm-shifting solutions to improve the outcome of diagnosis and therapy for patients suffering from cancer and other diseases. Nanomedicine, or the use of nanoscale (10-200 nm) constructs for therapeutic delivery, is emerging as a powerful tool in cancer care. Significant advances in nanomaterials and nanotechnology have paved the way for several carriers, such as dendrimers, liposomes and polymeric micelles, for clinical use. The goal is to enhance the safety and efficacy of therapeutic agents through encapsulation or other attachment (covalent or non-covalent) with carriers to form nanoparticles (NP). Some advantages afforded for drug delivery using NP include prolonged blood circulation time, increased loading capacity, improved stability and slower release time of the drug or active agent.

NP have been designed as nanocarriers to improve the delivery of therapeutic and imaging agents, but have thus far met with limited success (see for example, (Bae and Park, 2011; Chen et al., 2014; Lazarovits et al., 2015; Min et al., 2015; Park, 2013; Wang et al., 2012; Wilhelm et al., 2016; Wolfbeis, 2015; Xu et al., 2017). Multi-functional NP for multi-modality imaging and therapy are especially suited for image-guided drug delivery (Cavalieri et al., 2010; Foy et al., 2010; Homan et al., 2010; Koning and Krijger, 2007; Myhr, 2007; Peng et al., 2011; Zhang et al., 2010). NP may be particularly useful for small, rapidly excreted agents by increasing their residence time in the circulation and thus, their opportunity to reach target tissue (Farokhzad and Langer, 2009). Although NP size helps reduce unwanted rapid clearance from the blood, its size greatly hinders transport across biological interfaces. Effective systemic nanodelivery and therapeutic efficacy are stymied by the vascular EC barrier and by the rapid and robust uptake by the reticuloendothelial system (RES) (Chrastina et al., 2011; Durymanov et al., 2015; Ernsting et al., 2013; Jain and Stylianopoulos, 2010; Moghimi and Hunter, 2001; Steichen et al., 2013; von Roemeling et al., 2017). In fact, the RES uptake of NP is by far more robust than uptake in tumors or other organs. It effectively competes with the desired targeted delivery. Nearly all NP have a natural tropism for liver, spleen and other RES organs that depend on size, coating, dose and other factors. There is a need to develop better NP-based targeting and delivery strategies to overcome in vivo barriers to improve efficiency, efficacy and reduce side effects (Anchordoquy et al., 2017; DawidczykKim et al., 2014; DawidczykRussell et al., 2014; Wilhelm et al., 2016).

We explored the feasibility of using the caveolae pumping system to provide an effective solution to the NP delivery problem. Exploiting the vascular EC caveolae to concentrate targeted NP and their pharmaceutically active cargo inside disease tissues offers the means to increase efficacy at significantly lower doses (as compared to the standard dose of the untargeted therapy required to achieve efficacy), restrict the site of action and drastically decrease side effects as a direct result of improved precision delivery. We generated a new class of caveolae-targeting nanotherapies by linking a tumor caveolae-targeting agent (i.e. mAnnA1 antibody) to NP carriers specifically designed to increase the payload capacity of the targeting antibody while also minimizing any size effects on the caveolae pumping system.

Our published and preliminary data using IVM, SPECT-CT, and EM show that the caveolae pumping system provides optimum tissue penetration for NP<20 nm in diameter (Chrastina et al., 2011; Chrastina et al., 2010; McIntosh et al., 2000; D. P. McIntosh et al., 2002; Oh et al., 2007; Oh et al., 2004; Oh et al., 2014; Schnitzer, 2001; Valadon et al., 2010). This previous work included examining the effects of different sizes of nanostructures such as gold particles and nanostreptabodies (biotin-engineered antibody fragments on a streptavidin scaffold with a defined capacity for additional biotinylated payloads). We focused on using NP<20 nm nanoparticle carriers, such as poly(amidoamine) (PAMAM) dendrimers to optimize transport.

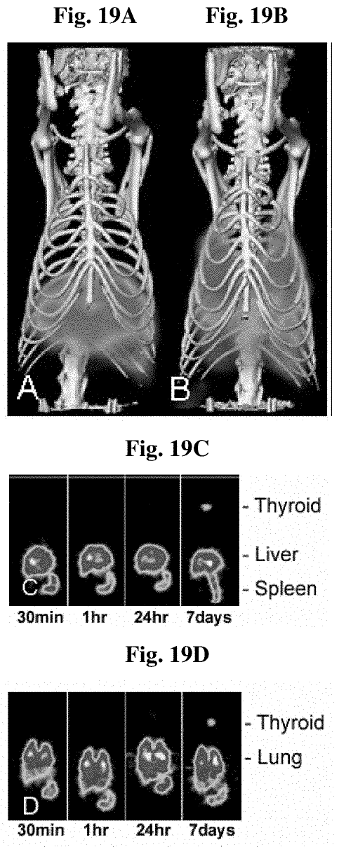

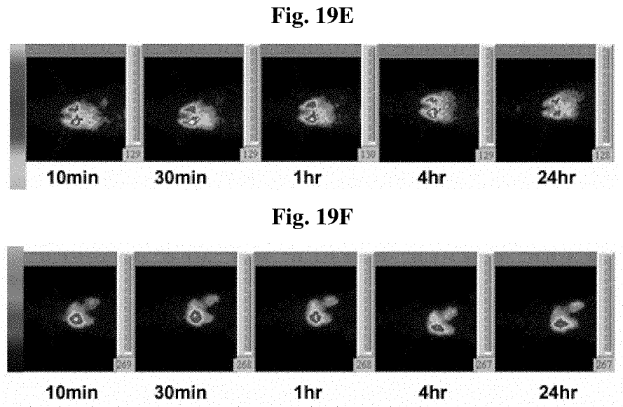

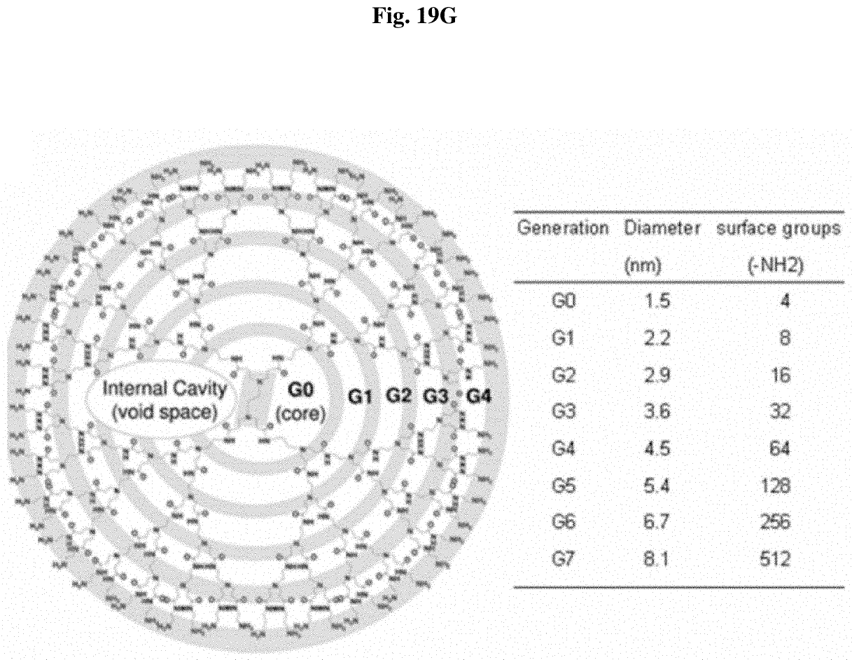

Poly(amidoamine) (PAMAM) dendrimers are the most extensively studied in their class (Tomalia, 1991; Tomalia et al., 2007; Wei et al., 2007) of hyper-branched, well-defined, monodisperse polymers with a highly uniform size and molecular weight. The abundance of terminal groups that exponentially increases with each generation (FIG. 19G) provides a large capacity for attachment of imaging agents and radiopharmaceuticals (Tomalia et al., 2007) (Kobayashi and Brechbiel, 2004). In fact, highly polyvalent surface of PAMAM capable of flexible derivatization was used to create high loads of various metallic imaging agents (Gd(3+) (Kobayashi et al., 2003; Kobayashi et al., 2007; Xu et al., 2007), and ⁹⁹mTc (Parrott et al., 2009). PAMAM scaffold was also used in VEGF-targeted boron neutron capture therapy (Backer et al., 2005). Thus, PAMAM dendrimers can be excellent carriers for radionuclides, toxic metals and other therapeutic agents.

We conjugated antibodies specific to aminopeptidase 2 (mAPP2), a target protein concentrated in lung vascular EC caveolae, directly to two different radiolabeled and uncloaked (non-PEGylated) dendrimers and assessed the ability of each caveolae-targeted immunoconjugate to target lung tissue after i.v. injection. Both γ-scintigraphy and SPECT-CT imaging showed robust lung uptake of both mAPP2-G5 and mAPP2-G4 PAMAM dendrimers (FIG. 19, panels B, D and E), whereas the control untargeted NP, as expected, accumulated rapidly in the liver and spleen (see panels A, C and F). Note both the caveolae pumping system in the lung and the RES of the liver and spleen were very robust, efficient and complete in accumulating their respective NPs. Most to nearly all of the uptake occurred for both in the first 30 min. The natural tropism of these uncloaked NP to the RES was largely avoided with conjugation to mAPP2. Thus, the caveolae pumping system can compete favorably with the RES and be robust enough to obviate RES uptake of NP and re-target them specifically to a single tissue (i.e. lung). Further optimization can reduce RES uptake by cloaking (PEGylation) of the NP.

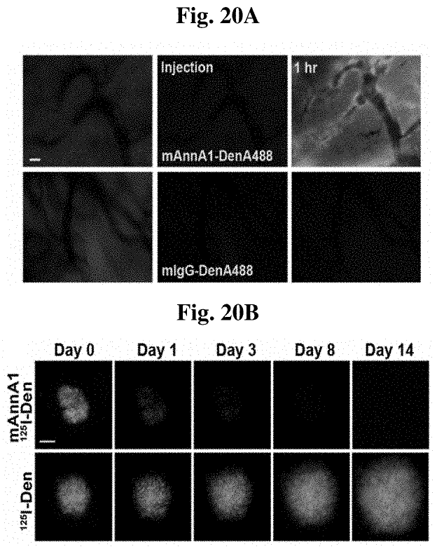

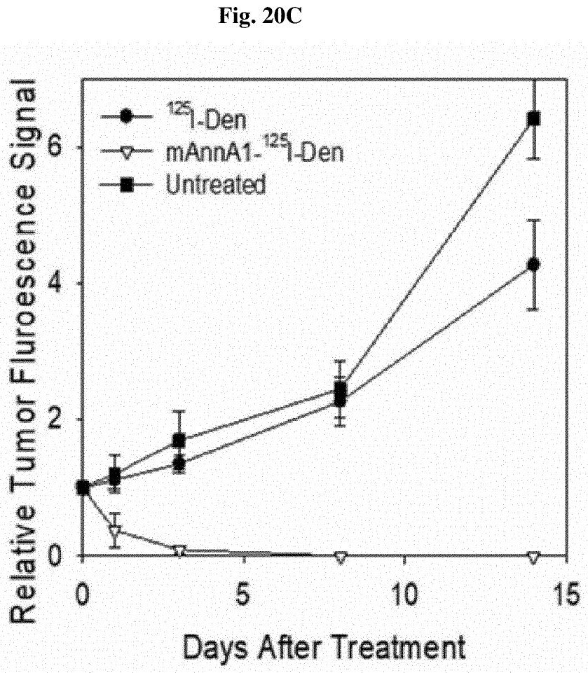

We next determined if using this prevision delivery strategy would work to concentrate NP loaded with ¹²⁵I directly into solid tumors and exert an enhanced therapeutic effect at low doses. We first conjugated fluorescently labeled A488 PAMAM G5 dendrimers to either mAnna1 or isotype matched IgG (control) antibodies to reach a 4 dendrimer per antibody ratio. We performed intravital microscopy (IVM) on orthotopic mammary tumor models following tail vein injection of 3 μg of each conjugate. Captured images (FIG. 20) show excellent tumor uptake with strong EC surface binding and ample transendothelial transport flooding the tumor, as evident by the strong fluorescent signal in tumor interstitium at 1 hr for mAnnA1-dendrimers; note the lack of signal from IgG-dendrimer-A488 control antibodies. We then evaluated the therapeutic efficacy using the same 4:1 nanoconstruct, but this time carrying not a fluorophore, but rather a single radionuclide ¹²⁵I per dendrimer. Following i.v. injection, IVM analysis showed a very strong anti-tumor response with ¹²⁵I-mAnnA-dendrimers, but not control ¹²⁵I-IgG-dendrimers (FIG. 20). These data demonstrate the feasibility of targeting NP carriers to caveolae to enhance both tumor precision delivery and therapeutic efficacy. It is also evident that the anti-tumor response requires the specificity and transport enabled by the caveolae pumping system to breach the vascular endothelial barrier and deliver the toxic agent directly to the site of diseased tissue to exert maximal effect at low doses.

By way of further example, the antibody-drug conjugate as described in Example 3 that we generated using carboxymethyl dextran (CMD) as a carrier for cisplatin was able to successfully eradicate tumors at ultra low doses as the result of improved targeted drug delivery. The free carboxylic acid groups of the dextran molecule make it possible to chelate cisplatin, as well as other toxic metals with therapeutic benefit (e.g. ¹⁷⁷Lu, ²²⁵Ac, ²²¹Fr and ²¹³Bi) to this polymeric linker. These loaded CMD polymeric structures are small enough to be considered as nanocarriers, like the PAMAM dendrimers described above, to enhance delivery and potency of low doses of therapeutic agents when targeted for transvascular transport via the caveolae pumping system.

Together, these results show that linking caveolae-targeting antibodies to loaded NP carriers can increase the capacity of antibodies and other targeting agents to deliver ultra low doses of small toxic agents (such as radionuclides, metals and small molecule drugs) into diseased tissue for enhanced therapeutic efficacy.

All of the compositions, articles, devices, systems, and methods disclosed and claimed herein can be made and executed without undue experimentation in light of the present disclosure. While the compositions, articles, devices, systems, and methods of this invention have been described in terms of preferred embodiments, it will be apparent to those of skill in the art that variations may be applied without departing from the spirit and scope of the invention. All such variations and equivalents apparent to those skilled in the art, whether now existing or later developed, are deemed to be within the spirit and scope of the invention as defined by the appended claims.

All patents, patent applications, and publications mentioned in the specification are indicative of the levels of those of ordinary skill in the art to which the invention pertains. All patents, patent applications, and publications are herein incorporated by reference in their entirety for all purposes and to the same extent as if each individual publication was specifically and individually indicated to be incorporated by reference in its entirety for any and all purposes.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modifications, and variations of the concepts herein described may be resorted to by those skilled in the art, and that such modifications and variations are considered to be within the scope of this invention as defined by the appended claims.

### REFERENCES

Anchordoquy, T. J., Barenholz, Y., Boraschi, D., Chorny, M., Decuzzi, P., Dobrovolskaia, M. A., . . . Simberg, D. (2017). Mechanisms and Barriers in Cancer Nanomedicine: Addressing Challenges, Looking for Solutions. ACS Nano, 11(1), 12-18. doi:10.1021/acsnano.6b08244
Anscher, M. S., Thrasher, B., Rabbani, Z., Teicher, B., & Vujaskovic, Z. (2006). Antitransforming growth factor-beta antibody 1D11 ameliorates normal tissue damage caused by high-dose radiation. Int J Radiat Oncol Biol Phys, 65(3), 876-881. doi:10.1016/j.ijrobp.2006.02.051
Asaishi, K., Endrich, B., Gotz, A., & Messmer, K. (1981). Quantitative analysis of microvascular structure and function in the amelanotic melanoma A-Mel-3. Cancer Res, 41(5), 1898-1904.
Ask, K., Martin, G. E., Kolb, M., & Gauldie, J. (2006). Targeting genes for treatment in idiopathic pulmonary fibrosis: challenges and opportunities, promises and pitfalls. Proc Am Thorac Soc, 3(4), 389-393.
Babin, A. L., Cannet, C., Gerard, C., Wyss, D., Page, C. P., & Beckmann, N. (2011). Noninvasive assessment of bleomycin-induced lung injury and the effects of short-term glucocorticosteroid treatment in rats using MRI. J Magn Reson Imaging, 33(3), 603-614. doi:10.1002/jmri.22476
Backer, M. V., Gaynutdinov, T. I., Patel, V., Bandyopadhyaya, A. K., Thirumamagal, B. T., Tjarks, W., . . . Backer, J. M. (2005). Vascular endothelial growth factor selectively targets boronated dendrimers to tumor vasculature. Mol Cancer Ther, 4(9), 1423-1429. doi:10.1158/1535-7163.mct-05-0161
Bae, Y. H., & Park, K. (2011). Targeted drug delivery to tumors: myths, reality and possibility. J Control Release, 153(3), 198-205. doi:10.1016/j.jconrel.2011.06.001
Blaney Davidson, E. N., van der Kraan, P. M., & van den Berg, W. B. (2007). TGF-beta and osteoarthritis. Osteoarthritis Cartilage, 15(6), 597-604.
Bommareddy, P. K., Silk Aw Fau-Kaufman, H. L., Kaufman, H. L., Gupta, A., Kaur, C. D., Saraf, S., . . . Gilbert, M. R. Intratumoral Approaches for the Treatment of Melanoma. (1540-336X (Electronic)).
Borgstrom, P., Oh, P., Czarny, M., Racine, B., & Schnitzer, J. E. (2013). Co-implanting orthotopic tissue creates stroma microenvironment enhancing growth and angiogenesis of multiple tumors. F1000Res, 2, 129. doi:10.12688/f1000research.2-129.v2
Brown K K, F. K., Daniels C, et al., (2008). Safety and tolerability of GC1008, a human monoclonal antibody against TGFbeta, in patients with idiopathic pulmonary fibrosis . . . Am J Respir Crit Care Med., 177.
Brown, L. F., Harrist, T. J., Yeo, K. T., Stahle-Backdahl, M., Jackman, R. W., Berse, B., . . . Detmar, M. (1995). Increased expression of vascular permeability factor (vascular endothelial growth factor) in bullous pemphigoid, dermatitis herpetiformis, and erythema multiforme. J Invest Dermatol, 104(5), 744-749.
Brown, L. F., Yeo, K. T., Berse, B., Morgentaler, A., Dvorak, H. F., & Rosen, S. (1995). Vascular permeability factor (vascular endothelial growth factor) is strongly expressed in the normal male genital tract and is present in substantial quantities in semen. J Urol, 154(2 Pt 1), 576-579.
Burris, H. A., 3rd, Tibbitts, J., Holden, S. N., Sliwkowski, M. X., & Lewis Phillips, G. D. (2011). Trastuzumab emtansine (T-DM1): a novel agent for targeting HER2+ breast cancer. Clin Breast Cancer, 11(5), 275-282. doi:10.1016/j.clbc.2011.03.018
Burrows, F. J., & Thorpe, P. E. (1993). Eradication of large solid tumors in mice with an immunotoxin directed against tumor vasculature. Proc Natl Acad Sci USA, 90, 8996-9000.
Carver, L. A., & Schnitzer, J. E. (2002). Tissue-specific pharmacodelivery and overcoming key cell barriers in vivo: Vascular targeting of caveolae. In V. Muzykantov & B. Torchilin (Eds.), Biomedical Aspects of Drug Targeting (pp. 107-128). Boston: Kluwer Academic Publishers.
Carver, L. A., & Schnitzer, J. E. (2003). Caveolae: mining little caves for new cancer targets. Nat Rev Cancer, 3(8), 571-581. doi:10.1038/nrc1146
Carver, L. A., & Schnitzer, J. E. (2007a). Multiple Functions and Clinical Uses of Caveolae in Endothelium. In W. C. Aird (Ed.), Endothelial Biomedicine (pp. 664-678): Cambridge University Press.
Carver, L. A., & Schnitzer, J. E. (2007b). Proteomic Mapping of Endothelium and Vascular Targeting in Vivo. In W. C. Aird (Ed.), Endothelial Biomedicine (pp. 881-898).
Carver, L. A., Schnitzer, J. E., Anderson, R. G., & Mohla, S. (2003). Role of caveolae and lipid rafts in cancer: workshop summary and future needs. Cancer Res, 63(20), 6571-6574.
Cavalieri, F., Zhou, M., & Ashokkumar, M. (2010). The design of multifunctional microbubbles for ultrasound image-guided cancer therapy. Curr Top Med Chem, 10(12), 1198-1210.
Chari, R. V., Martell, B. A., Gross, J. L., Cook, S. B., Shah, S. A., Blattler, W. A., . . . Goldmacher, V. S. (1992). Immunoconjugates containing novel maytansinoids: promising anticancer drugs. Cancer Res, 52(1), 127-131.
Chari, R. V., Miller, M. L., & Widdison, W. C. (2014). Antibody-drug conjugates: an emerging concept in cancer therapy. Angew Chem Int Ed Engl, 53(15), 3796-3827. doi:10.1002/anie.201307628
Chen, F., Ehlerding, E. B., & Cai, W. (2014). Theranostic nanoparticles. J Nucl Med, 55(12), 1919-1922. doi:10.2967/jnumed.114.146019
Chrastina, A., Massey, K. A., & Schnitzer, J. E. (2011). Overcoming in vivo barriers to targeted nanodelivery. Wiley Interdiscip Rev Nanomed Nanobiotechnol, 3(4), 421-437. doi:10.1002/wnan.143
Chrastina, A., & Schnitzer, J. E. (2010). 125-Iodine radiolabeling of silver nanoparticles for in vivo SPECT imaging. Int J Nanomed, 5, 653-659.
Chrastina, A., Valadon, P., Massey, K. A., & Schnitzer, J. E. (2010). Lung Vascular Targeting Using Antibody to Aminopeptidase P: CT-SPECT Imaging, Biodistribution and Pharmacokinetic Analysis. J Vasc Res, 47(6), 531-543.
Dawidczyk, C. M., Kim, C., Park, J. H., Russell, L. M., Lee, K. H., Pomper, M. G., & Searson, P. C. (2014). State-of-the-art in design rules for drug delivery platforms: lessons learned from FDA-approved nanomedicines. J Control Release, 187, 133-144. doi:10.1016/j.jconrel.2014.05.036
Dawidczyk, C. M., Russell, L. M., & Searson, P. C. (2014). Nanomedicines for cancer therapy: state-of-the-art and limitations to pre-clinical studies that hinder future developments. Front Chem, 2, 69. doi:10.3389/fchem.2014.00069
Deng, S., Wang, J., Hou, L., Li, J., Chen, G., Jing, B., . . . Yang, Z. (2013). Annexin A1, A2, A4 and A5 play important roles in breast cancer, pancreatic cancer and laryngeal carcinoma, alone and/or synergistically. Oncol Lett, 5(1), 107-112. doi:10.3892/ol.2012.959
Denton, C. P., Merkel, P. A., Furst, D. E., Khanna, D., Emery, P., Hsu, V. M., . . . Seibold, J. R. (2007). Recombinant human anti-transforming growth factor beta1 antibody therapy in systemic sclerosis: a multicenter, randomized, placebo-controlled phase I/II trial of CAT-192. Arthritis Rheum, 56(1), 323-333.
du Bois, R. M. (2010). Strategies for treating idiopathic pulmonary fibrosis. Nat Rev Drug Discov, 9(2), 129-140.
Durr, E., Yu, J., Krasinska, K. M., Carver, L. A., Yates, J. R., Testa, J. E., . . . Schnitzer, J. E. (2004). Direct proteomic mapping of the lung microvascular endothelial cell surface in vivo and in cell culture. Nat Biotechnol, 22(8), 985-992. doi:10.1038/nbt993
Durymanov, M. O., Rosenkranz, A. A., & Sobolev, A. S. (2015). Current Approaches for Improving Intratumoral Accumulation and Distribution of Nanomedicines. Theranostics, 5(9), 1007-1020. doi:10.7150/thno.11742
Dvorak, H. F. (2002). Vascular permeability factor/vascular endothelial growth factor: a critical cytokine in tumor angiogenesis and a potential target for diagnosis and therapy. J Clin Oncol, 20(21), 4368-4380.
Dvorak, H. F., Brown, L. F., Detmar, M., & Dvorak, A. M. (1995). Vascular permeability factor/vascular endothelial growth factor, microvascular hyperpermeability, and angiogenesis. Am J Pathol, 146(5), 1029-1039.
Dvorak, H. F., Nagy, J. A., & Dvorak, A. M. (1991). Structure of solid tumors and their vasculature: implications for therapy with monoclonal antibodies. Cancer Cells, 3, 77-85.
Dvorak, H. F., Nagy, J. A., Dvorak, J. T., & Dvorak, A. M. (1988). Identification and characterization of the blood vessels of solid tumors that are leaky to circulating macromolecules. Am. J. Pathol., 133, 95.
Dvorak, H. F., Nagy, J. A., Feng, D., Brown, L. F., & Dvorak, A. M. (1999). Vascular permeability factor/vascular endothelial growth factor and the significance of microvascular hyperpermeability in angiogenesis. Curr Top Microbiol Immunol, 237, 97-132.
Dvorak, H. F., Sioussat, T. M., Brown, L. F., Berse, B., Nagy, J. A., Sotrel, A., . . . Senger, D. R. (1991). Distribution of vascular permeability factor (vascular endothelial growth factor) in tumors: concentration in tumor blood vessels. J Exp Med, 174(5), 1275-1278.
Edwards, D. R., Murphy, G., Reynolds, J. J., Whitham, S. E., Docherty, A. J., Angel, P., & Heath, J. K. (1987). Transforming growth factor beta modulates the expression of collagenase and metalloproteinase inhibitor. Embo J, 6(7), 1899-1904.
Endrich, B., Asaishi, K., Gotz, A., & Messmer, K. (1980). Technical report—a new chamber technique for microvascular studies in unanesthetized hamsters. Res Exp Med (Berl), 177(2), 125-134.
Ernsting, M. J., Murakami, M., Roy, A., & Li, S. D. (2013). Factors controlling the pharmacokinetics, biodistribution and intratumoral penetration of nanoparticles. J Control Release, 172(3), 782-794. doi:10.1016/j.jconrel.2013.09.013
Farokhzad, O. C., & Langer, R. (2009). Impact of nanotechnology on drug delivery. ACS Nano, 3(1), 16-20.
Flanders, K. C. (2004). Smad3 as a mediator of the fibrotic response. Int J Exp Pathol, 85(2), 47-64.
Foy, S. P., Manthe, R. L., Foy, S. T., Dimitrijevic, S., Krishnamurthy, N., & Labhasetwar, V. (2010). Optical imaging and magnetic field targeting of magnetic nanoparticles in tumors. ACS Nano, 4(9), 5217-5224. doi:10.1021/nn101427t
Frentzas, S., Simoneau, E., Bridgeman, V. L., Vermeulen, P. B., Foo, S., Kostaras, E., . . . Reynolds, A. R. (2016). Vessel co-option mediates resistance to anti-angiogenic therapy in liver metastases. Nat Med, 22(11), 1294-1302. doi:10.1038/nm.4197
Gerke, V., & Moss, S. E. (2002). Annexins: from structure to function. Physiol Rev, 82(2), 331-371
Giri, S. N., Hyde, D. M., & Hollinger, M. A. (1993). Effect of antibody to transforming growth factor beta on bleomycin induced accumulation of lung collagen in mice. Thorax, 48(10), 959-966.
Griffin, N. M., & Schnitzer, J. E. (2008). Chapter 8. Proteomic mapping of the vascular endothelium in vivo for vascular targeting. Methods Enzymol, 445, 177-208.
Griffiths, G. L., Mattes, M. J., Stein, R., Govindan, S. V., Horak, I. D., Hansen, H. J., & Goldenberg, D. M. (2003). Cure of SCID mice bearing human B-lymphoma xenografts by an anti-CD74 antibody-anthracycline drug conjugate. Clin Cancer Res, 9(17), 6567-6571.
Gupta, A., Kaur, C. D., Saraf, S., Saraf, S., Kota, J., Hancock, J., . . . Gilbert, M. R. Targeting of herbal bioactives through folate receptors: a novel concept to enhance intracellular drug delivery in cancer therapy
Pancreatic cancer: Stroma and its current and emerging targeted therapies. LID-S0304-3835(17)30014-9 [pii] LID—10.1016/j.canlet.2016.12.035 [doi]
Toward Personalized Targeted Therapeutics: An Overview. (1532-4281 (Electronic)).
Guy, C. T., Cardiff, R. D., & Muller, W. J. (1992). Induction of mammary tumors by expression of polyomavirus middle T oncogene: a transgenic mouse model for metastatic disease. Mol Cell Biol, 12(3), 954-961.
Guy, C. T., Webster, M. A., Schaller, M., Parsons, T. J., Cardiff, R. D., & Muller, W. J. (1992). Expression of the neu protooncogene in the mammary epithelium of transgenic mice induces metastatic disease. Proc Natl Acad Sci USA, 89(22), 10578-10582.
Hajitou, A., Pasqualini, R., & Arap, W. (2006). Vascular targeting: recent advances and therapeutic perspectives. Trends Cardiovasc Med, 16(3), 80-88.
Homan, K., Shah, J., Gomez, S., Gensler, H., Karpiouk, A., Brannon-Peppas, L., & Emelianov, S. (2010). Silver nanosystems for photoacoustic imaging and image-guided therapy. J Biomed Opt, 15(2), 021316. doi:10.1117/1.3365937
Huang, X., Molema, G., King, S., Watkins, L., Edgington, T. S., & Thorpe, P. E. (1997). Tumor infarction in mice by antibody-directed targeting of tissue factor to tumor vasculature. Science, 275, 547-550.
Ignotz, R. A., & Massague, J. (1986). Transforming growth factor-beta stimulates the expression of fibronectin and collagen and their incorporation into the extracellular matrix. J Biol Chem, 261(9), 4337-4345.
Jain, R. K. (2002). Tumor angiogenesis and accessibility: role of vascular endothelial growth factor. Semin Oncol, 29(6 Suppl 16), 3-9.
Jain, R. K., & Stylianopoulos, T. (2010). Delivering nanomedicine to solid tumors. Nat Rev Clin Oncol, 7(11), 653-664. doi:10.1038/nrclinonc.2010.139
Kaartinen, V., Voncken, J. W., Shuler, C., Warburton, D., Bu, D., Heisterkamp, N., & Groffen, J. (1995). Abnormal lung development and cleft palate in mice lacking TGF-beta 3 indicates defects of epithelial-mesenchymal interaction. Nat Genet, 11(4), 415-421.
Kaminski, N., Allard, J. D., Pittet, J. F., Zuo, F., Griffiths, M. J., Morris, D., . . . Heller, R. A. (2000). Global analysis of gene expression in pulmonary fibrosis reveals distinct programs regulating lung inflammation and fibrosis. Proc Natl Acad Sci USA, 97(4), 1778-1783.
Kanda, T., Sullivan, K. F., & Wahl, G. M. (1998). Histone-GFP fusion protein enables sensitive analysis of chromosome dynamics in living mammalian cells. Curr Biol, 8(7), 377-385.
Khaw, P., Grehn, F., Hollo, G., Overton, B., Wilson, R., Vogel, R., & Smith, Z. (2007). A phase III study of subconjunctival human anti-transforming growth factor beta(2) monoclonal antibody (CAT-152) to prevent scarring after first-time trabeculectomy. Ophthalmology, 114(10), 1822-1830.
Kim, J. H., Kim, H. Y., Kim, S., Chung, J. H., Park, W. S., & Chung, D. H. (2005). Natural killer T (NKT) cells attenuate bleomycin-induced pulmonary fibrosis by producing interferon-gamma. Am J Pathol, 167(5), 1231-1241.
Kobayashi, H., & Brechbiel, M. W. (2004). Dendrimer-based nanosized MRI contrast agents. Curr Pharm Biotechnol, 5(6), 539-549.
Kobayashi, H., Kawamoto, S., Star, R. A., Waldmann, T. A., Tagaya, Y., & Brechbiel, M. W. (2003). Micro-magnetic resonance lymphangiography in mice using a novel dendrimer-based magnetic resonance imaging contrast agent. Cancer Res, 63(2), 271-276.
Kobayashi, H., Koyama, Y., Barrett, T., Hama, Y., Regino, C. A., Shin, I. S., . . . Urano, Y. (2007). Multimodal nanoprobes for radionuclide and five-color near-infrared optical lymphatic imaging. ACS Nano, 1(4), 258-264. doi:10.1021/nn700062z
Konigshoff, M., Kramer, M., Balsara, N., Wilhelm, J., Amarie, 0. V., Jahn, A., . . . Eickelberg, O. (2009). WNT1-inducible signaling protein-1 mediates pulmonary fibrosis in mice and is upregulated in humans with idiopathic pulmonary fibrosis. J Clin Invest, 119(4), 772-787. doi:33950 [pii]
10.1172/JCI33950
Koning, G. A., & Krijger, G. C. (2007). Targeted multifunctional lipid-based nanocarriers for image-guided drug delivery. Anticancer Agents Med Chem, 7(4), 425-440.
Kulkarni, A. B., Huh, C. G., Becker, D., Geiser, A., Lyght, M., Flanders, K. C., . . . Karlsson, S. (1993). Transforming growth factor beta 1 null mutation in mice causes excessive inflammatory response and early death. Proc Natl Acad Sci USA, 90(2), 770-774.
Laudanna, C., & Constantin, G. (2003). New models of intravital microscopy for analysis of chemokine receptor-mediated leukocyte vascular recognition. J Immunol Methods, 273(1-2), 115-123.
Lazarovits, J., Chen, Y. Y., Sykes, E. A., & Chan, W. C. (2015). Nanoparticle-blood interactions: the implications on solid tumour targeting. Chem Commun (Carob), 51(14), 2756-2767. doi:10.1039/c4cc07644c
Lazenby, A. J., Crouch, E. C., McDonald, J. A., & Kuhn, C., 3rd. (1990). Remodeling of the lung in bleomycin-induced pulmonary fibrosis in the rat. An immunohistochemical study of laminin, type IV collagen, and fibronectin. Am Rev Respir Dis, 142(1), 206-214. doi:10.1164/ajrccm/142.1.206
Li, M., Krishnaveni, M. S., Li, C., Zhou, B., Xing, Y., Banfalvi, A., . . . Minoo, P. (2011). Epithelium-specific deletion of TGF-beta receptor type II protects mice from bleomycin-induced pulmonary fibrosis. J Clin Invest, 121(1), 277-287. doi:10.1172/jci42090
Li, Y., Yu, J., Wang, Y. Z., Griffin, N. M., Long, F., Shore, S., . . . Schnitzer, J. E. (2009). Enhancing identifications of lipid-embedded proteins by mass spectrometry for improved mapping of endothelial plasma membranes in vivo. Mol Cell Proteomics, Epub ahead of print.
Maher, T. M., Wells, A. U., & Laurent, G. J. (2007). Idiopathic pulmonary fibrosis: multiple causes and multiple mechanisms? Eur Respir J, 30(5), 835-839.
Martin, J. S., Dickson, M. C., Cousins, F. M., Kulkarni, A. B., Karlsson, S., & Akhurst, R. J. (1995). Analysis of homozygous TGF beta 1 null mouse embryos demonstrates defects in yolk sac vasculogenesis and hematopoiesis. Ann NY Acad Sci, 752, 300-308.
Massey, K. A., & Schnitzer, J. E. (2009). Targeting and imaging signature caveolar molecules in lungs. Proc Am Thorac Soc, 6(5), 419-430.
McIntosh, D. P., Tan, X.-Y., Oh, P., & Schnitzer, J. E. (2000). Targeting endothelial caveolae for specific transcytosis in vivo. FASEB J., 14, A26.
McIntosh, D. P., Tan, X.-Y., Oh, P., & Schnitzer, J. E. (2002). Targeting endothelium and its dynamic caveolae for tissue-specific transcytosis in vivo: A pathway to overcome cell barriers to drug and gene delivery. Proc. Natl. Acad. Sci. USA, 99, 1996-2001.
McIntosh, D. P., Tan, X. Y., Oh, P., & Schnitzer, J. E. (2002). Targeting endothelium and its dynamic caveolae for tissue-specific transcytosis in vivo: a pathway to overcome cell barriers to drug and gene delivery. Proc Natl Acad Sci USA, 99(4), 1996-2001. doi:10.1073/pnas.251662398
99/4/1996 [pii]
Min, Y., Caster, J. M., Eblan, M. J., & Wang, A. Z. (2015). Clinical Translation of Nanomedicine. Chem Rev, 115(19), 11147-11190. doi:10.1021/acs.chemrev.5b00116
Moghimi, S. M., & Hunter, A. C. (2001). Capture of stealth nanoparticles by the body's defences. Crit Rev Ther Drug Carrier Syst, 18(6), 527-550.
Myhr, G. (2007). Multimodal ultrasound mediated drug release model in local cancer therapy. Med Hypotheses, 69(6), 1325-1333. doi:10.1016/j.mehy.2007.03.006
Neri, A., Welch, D., Kawaguchi, T., & Nicolson, G. L. (1982). Development and biologic properties of malignant cell sublines and clones of a spontaneously metastasizing rat mammary adenocarcinoma. J Natl Cancer Inst, 68(3), 507-517.
Oh, P., Borgstrom, P., Witkiewicz, H., Li, Y., Borgstrom, B. J., Chrastina, A., . . . Schnitzer, J. E. (2007). Live dynamic imaging of caveolae pumping targeted antibody rapidly and specifically across endothelium in the lung. Nat Biotechnol, 25(3), 327-337. doi:10.1038/nbt1292
Oh, P., Li, Y., Yu, J. Y., Durr, E., Krasinska, K. M., Carver, L. A., . . . Schnitzer, J. E. (2004). Subtractive proteomic mapping of the endothelial surface in lung and solid tumours for tissue-specific therapy. Nature, 429(6992), 629-635.
Oh, P., McIntosh, D. P., & Schnitzer, J. E. (1997). Dynamin at the neck of caveolae mediates their internalization by GTP-dependent fission from the plasma membrane of endothelium. Mol. Biol. Cell, 8, 425a.
Oh, P., Testa, J. E., Borgstrom, P., Witkiewicz, H., Li, Y., & Schnitzer, J. E. (2014). In vivo proteomic imaging analysis of caveolae reveals pumping system to penetrate solid tumors. Nat Med, 20(9), 1062-1068. doi:10.1038/nm.3623
Park, K. (2013). Facing the truth about nanotechnology in drug delivery. ACS Nano, 7(9), 7442-7447. doi:10.1021/nn404501g
Parrott, M. C., Benhabbour, S. R., Saab, C., Lemon, J. A., Parker, S., Valliant, J. F., & Adronov, A. (2009). Synthesis, radiolabeling, and bio-imaging of high-generation polyester dendrimers. J Am Chem Soc, 131(8), 2906-2916. doi:10.1021/ja8078175
Peng, C. L., Shih, Y. H., Lee, P. C., Hsieh, T. M., Luo, T. Y., & Shieh, M. J. (2011). Multimodal image-guided photothermal therapy mediated by 188Re-labeled micelles containing a cyanine-type photosensitizer. ACS Nano, 5(7), 5594-5607. doi:10.1021/nn201100m
Pittet, J. F., Griffiths, M. J., Geiser, T., Kaminski, N., Dalton, S. L., Huang, X., . . . Sheppard, D. (2001). TGF-beta is a critical mediator of acute lung injury. J Clin Invest, 107(12), 1537-1544. doi:10.1172/jci11963
Postlethwaite, A. E., Keski-Oja, J., Moses, H. L., & Kang, A. H. (1987). Stimulation of the chemotactic migration of human fibroblasts by transforming growth factor beta. J Exp Med, 165(1), 251-256.
Prud'homme, G. J. (2007). Pathobiology of transforming growth factor beta in cancer, fibrosis and immunologic disease, and therapeutic considerations. Lab Invest, 87(11), 1077-1091.
Quaglino, E., Mastini, C., Forni, G., & Cavallo, F. (2008). ErbB2 transgenic mice: a tool for investigation of the immune prevention and treatment of mammary carcinomas. Curr Protoc Immunol, Chapter 20, Unit 20 29 21-20 29-10.
Raghu, G., Masta, S., Meyers, D., & Narayanan, A. S. (1989). Collagen synthesis by normal and fibrotic human lung fibroblasts and the effect of transforming growth factor-beta. Am Rev Respir Dis, 140(1), 95-100.
Reilly, R. T., Gottlieb, M. B., Ercolini, A. M., Machiels, J. P., Kane, C. E., Okoye, F. I., . . . Jaffee, E. M. (2000). HER-2/neu is a tumor rejection target in tolerized HER-2/neu transgenic mice. Cancer Res, 60(13), 3569-3576.
Ruoslahti, E., Bhatia, S. N., & Sailor, M. J. (2010). Targeting of drugs and nanoparticles to tumors. J Cell Biol, 188(6), 759-768. doi:10.1083/jcb.200910104
Sanford, L. P., Ormsby, I., Gittenberger-de Groot, A. C., Sariola, H., Friedman, R., Boivin, G. P., . . . Doetschman, T. (1997). TGFbeta2 knockout mice have multiple developmental defects that are non-overlapping with other TGFbeta knockout phenotypes. Development, 124(13), 2659-2670.
Schechter, B., Pauzner, R., Arnon, R., Haimovich, J., & Wilchek, M. (1987). Selective cytotoxicity against tumor cells by cisplatin complexed to antitumor antibodies via carboxymethyl dextran. Cancer Immunology, Immunotherapy, 25(3), 225-230.
Schechter, B., Wilchek, M., & Arnon, R. (1987). Increased therapeutic efficacy of cis-platinum complexes of poly-L-glutamic acid against a murine carcinoma. Int J Cancer, 39(3), 409-413
Schnitzer, J. E. (1997a). The endothelial cell surface and caveolae in health and disease. In G. V. R. Born & C. J. Schwartz (Eds.), Vascular Endothelium: Physiology, pathology and therapeutic opportunities. (pp. 77-95). Stuttgart: Schattauer.
Schnitzer, J. E. (1997b). Transport functions of the glycocalyx, specific proteins, and caveolae in endothelium. In J. Bassingthwaite, C. A. Goresky, & J. H. Linehan (Eds.), Capillary Permeation, Cellular Transport and Reaction Kinetics (pp. 31-69). London: Oxford Press.
Schnitzer, J. E. (1998). Vascular targeting as a strategy for cancer therapy. N Engl J Med, 339(7), 472-474.
Schnitzer, J. E. (2001). Caveolae: from basic trafficking mechanisms to targeting transcytosis for tissue-specific drug and gene delivery in vivo. Adv Drug Deliv Rev, 49(3), 265-280.
Schnitzer, J. E., Liu, J., & Oh, P. (1995). Endothelial caveolae have the molecular transport machinery for vesicle budding, docking, and fusion including VAMP, NSF, SNAP, annexins, and GTPases. J Biol Chem, 270(24), 14399-14404.
Schnitzer, J. E., McIntosh, D. P., Dvorak, A. M., Liu, J., & Oh, P. (1995). Separation of caveolae from associated microdomains of GPI-anchored proteins. Science, 269, 1435-1439.
Schnitzer, J. E., & Oh, P. (1994). Albondin-mediated capillary permeability to albumin. Differential role of receptors in endothelial transcytosis and endocytosis of native and modified albumins. J Biol Chem, 269(8), 6072-6082.
Schnitzer, J. E., & Oh, P. (1995). Caveolae are dynamic carriers that bud from plasma membranes in a GTP-dependent manner. Mol. Biol. Cell, 6, 402a.
Schnitzer, J. E., Oh, P., Jacobson, B. S., & Dvorak, A. M. (1994). Caveolin-enriched caveolae purified from endothelium in situ are transport vesicles for albondin-mediateed transcytosis of albumin. Mol Biol Cell, 5, A75.
Schnitzer, J. E., Oh, P., Jacobson, B. S., & Dvorak, A. M. (1995). Caveolae from luminal plasmalemma of rat lung endothelium: microdomains enriched in caveolin, Ca(2+)-ATPase, and inositol trisphosphate receptor. Proc Natl Acad Sci USA, 92(5), 1759-1763.
Schnitzer, J. E., Oh, P., & McIntosh, D. P. (1996). Role of GTP hydrolysis in fission of caveolae directly from plasma membranes [publisher's erratum appears in Science 1996 Nov. 15; 274(5290):1069]. Science, 274(5285), 239-242.
Shaker, O. G., & Sourour, D. A. (2011). Effect of leukotriene receptor antagonists on lung fibrosis in rats. J Appl Toxicol, 31(7), 678-684. doi:10.1002/jat.1625
Shao, S., Tsai, M. H., Lu, J., Yu, T., Jin, J., Xiao, D., . . . Wang, J. (2018). Site-specific and hydrophilic ADCs through disulfide-bridged linker and branched PEG. Bioorg Med Chem Lett, 28(8), 1363-1370. doi:10.1016/j.bmcl.2018.03.005
Shen, D., Chang, H. R., Chen, Z., He, J., Lonsberry, V., Elshimali, Y., . . . Gornbein, J. A. (2005). Loss of annexin A1 expression in human breast cancer detected by multiple high-throughput analyses. Biochem Biophys Res Commun, 326(1), 218-227. doi:10.1016/j.bbrc.2004.10.214
Sime, P. J., & O'Reilly, K. M. (2001). Fibrosis of the lung and other tissues: new concepts in pathogenesis and treatment. Clin Immunol, 99(3), 308-319.
Sime, P. J., Xing, Z., Graham, F. L., Csaky, K. G., & Gauldie, J. (1997). Adenovector-mediated gene transfer of active transforming growth factor-beta1 induces prolonged severe fibrosis in rat lung. J Clin Invest, 100(4), 768-776.
Siriwardena, D., Khaw, P. T., King, A. J., Donaldson, M. L., Overton, B. M., Migdal, C., & Cordeiro, M. F. (2002). Human antitransforming growth factor beta(2) monoclonal antibody—a new modulator of wound healing in trabeculectomy: a randomized placebo controlled clinical study. Ophthalmology, 109(3), 427-431.
Sounni, N. E., Dehne, K., van Kempen, L., Egeblad, M., Affara, N. I., Cuevas, I., . . . Coussens, L. M. Stromal regulation of vessel stability by MMP14 and TGFbeta. Dis Model Mech, 3(5-6), 317-332.
Sousa, F., Castro, P., Fonte, P., Kennedy, P. J., Neves-Petersen, M. T., & Sarmento, B. (2016). Nanoparticles for the delivery of therapeutic antibodies: Dogma or promising strategy? Expert Opin Drug Deliv, 1-14. doi:10.1080/17425247.2017.1273345
Steichen, S. D., Caldorera-Moore, M., & Peppas, N. A. (2013). A review of current nanoparticle and targeting moieties for the delivery of cancer therapeutics. Eur J Pharm Sci, 48(3), 416-427. doi:10.1016/j.ejps.2012.12.006
Sykes, E. A., Chen, J., Zheng, G., & Chan, W. C. (2014). Investigating the impact of nanoparticle size on active and passive tumor targeting efficiency. ACS Nano, 8(6), 5696-5706. doi:10.1021/nn500299p
Tager, A. M., Kradin, R. L., LaCamera, P., Bercury, S. D., Campanella, G. S., Leary, C. P., . . . Luster, A. D. (2004). Inhibition of pulmonary fibrosis by the chemokine IP-10/CXCL10. Am J Respir Cell Mol Biol, 31(4), 395-404. doi:10.1165/rcmb.2004-01750C
Testa, J. E., Chrastina, A., Li, Y., Oh, P., & Schnitzer, J. E. (2009). Ubiquitous yet Distinct Expression of Podocalyxin on Vascular Surfaces in Normal and Tumor Tissues in the Rat. J Vasc Res, 46(4), 311-324.
Testa, J. E., Chrastina, A., Oh, P., Li, Y., Witkiewicz, H., Czarny, M., . . . Schnitzer, J. E. (2009). Immunotargeting and cloning of two CD34 variants exhibiting restricted expression in adult rat endothelia in vivo. Am J Physiol Lung Cell Mol Physiol, 297(2), L251-262. doi:10.1152/ajplung.90565.2008
Tomalia, D. A. (1991). Dendrimer research. Science, 252(5010), 1231. doi:10.1126/science.252.5010.1231-b
Tomalia, D. A., Reyna, L. A., & Svenson, S. (2007). Dendrimers as multi-purpose nanodevices for oncology drug delivery and diagnostic imaging. Biochem Soc Trans, 35(Pt 1), 61-67. doi:10.1042/bst0350061
Vajkoczy, P., Ullrich, A., & Menger, M. D. (2000). Intravital fluorescence videomicroscopy to study tumor angiogenesis and microcirculation. Neoplasia, 2(1-2), 53-61.
Valadon, P., Darsow, B., Buss, T. N., Czarny, M., Griffin, N. M., Nguyen, H. N., . . . Schnitzer, J. E. (2010). Designed auto-assembly of nanostreptabodies for rapid tissue-specific targeting in vivo. J Biol Chem, 285(1), 713-722. doi:10.1074/jbc.M109.061838
Valadon, P., Garnett, J. D., Testa, J. E., Bauerle, M., Oh, P., & Schnitzer, J. E. (2006). Screening phage display libraries for organ-specific vascular immunotargeting in vivo. Proc Natl Acad Sci USA, 103(2), 407-412.
von Roemeling, C., Jiang, W., Chan, C. K., Weissman, I. L., & Kim, B. Y. (2017). Breaking Down the Barriers to Precision Cancer Nanomedicine. Trends Biotechnol, 35(2), 159-171. doi:10.1016/j.tibtech.2016.07.006
Vyalov, S. L., Gabbiani, G., & Kapanci, Y. (1993). Rat alveolar myofibroblasts acquire alpha-smooth muscle actin expression during bleomycin-induced pulmonary fibrosis. Am J Pathol, 143(6), 1754-1765.
Wakankar, A. A., Feeney, M. B., Rivera, J., Chen, Y., Kim, M., Sharma, V. K., & Wang, Y. J. (2010). Physicochemical stability of the antibody-drug conjugate Trastuzumab-DM1: changes due to modification and conjugation processes. Bioconjug Chem, 21(9), 1588-1595. doi:10.1021/bc900434c
Wang, A. Z., Langer, R., & Farokhzad, 0. C. (2012). Nanoparticle delivery of cancer drugs. Annu Rev Med, 63, 185-198. doi:10.1146/annurev-med-040210-162544
Weathers, S. S., & Gilbert, M. R. (2016). Toward Personalized Targeted Therapeutics: An Overview. Neurotherapeutics. doi:10.1007/s13311-016-0496-5
Wei, C., Wei, W., Morris, M., Kondo, E., Gorbounov, M., & Tomalia, D. A. (2007). Nanomedicine and drug delivery. Med Clin North Am, 91(5), 863-870. doi:10.1016/j.mcna.2007.05.005
Wilhelm, S., Tavares, A. J., Dai, Q., Ohta, S., Audet, J., Dvorak, H. F., & Chan, W. C. W. (2016). Analysis of nanoparticle delivery to tumours. Nature Reviews Materials, 1(5), 16014. doi:10.1038/natrevmats.2016.14
Wilson, M. S., Madala, S. K., Ramalingam, T. R., Gochuico, B. R., Rosas, I. O., Cheever, A. W., & Wynn, T. A. (2010). Bleomycin and IL-1beta-mediated pulmonary fibrosis is IL-17A dependent. J Exp Med, 207(3), 535-552.
Wolfbeis, O. S. (2015). An overview of nanoparticles commonly used in fluorescent bioimaging. Chem. Soc. Rev., 44(14), 4743-4768. doi:10.1039/c4cs00392f
Xu, H., Regino, C. A., Bernardo, M., Koyama, Y., Kobayashi, H., Choyke, P. L., & Brechbiel, M. W. (2007). Toward improved syntheses of dendrimer-based magnetic resonance imaging contrast agents: new bifunctional diethylenetriaminepentaacetic acid ligands and nonaqueous conjugation chemistry. J. Med. Chem., 50(14), 3185-3193. doi:10.1021/jm061324m
Xu, J., Zhou, X., Li, Y., & Tian, Y. (2017). Cancer Nanotechnology: Recent trends and developments in strategies for targeting cancer cells to improve cancer imaging and treatment. Curr Drug Metab.
Yang, L., & Moses, H. L. (2008). Transforming growth factor beta: tumor suppressor or promoter? Are host immune cells the answer? Cancer Res, 68(22), 9107-9111.
Zhang, H. Y., Gharaee-Kermani, M., Zhang, K., Karmiol, S., & Phan, S. H. (1996). Lung fibroblast alpha-smooth muscle actin expression and contractile phenotype in bleomycin-induced pulmonary fibrosis. Am J Pathol, 148(2), 527-537.
Zhang, H. Y., & Phan, S. H. (1999). Inhibition of myofibroblast apoptosis by transforming growth factor beta(1). Am J Respir Cell Mol Biol, 21(6), 658-665.
Zhang, K., Flanders, K. C., & Phan, S. H. (1995). Cellular localization of transforming growth factor-beta expression in bleomycin-induced pulmonary fibrosis. Am J Pathol, 147(2), 352-361.
Zhang, L., Xu, J. S., Sanders, V. M., Letson, A. D., Roberts, C. J., & Xu, R. X. (2010). Multifunctional microbubbles for image-guided antivascular endothelial growth factor therapy. J Biomed Opt, 15(3), 030515. doi:10.1117/1.3457669
Zhu, D. Z., & Pauli, B. U. (1991). Generation of monoclonal antibodies directed against organ-specific endothelial cell surface determinants. J Histochem Cytochem, 39(8), 1137-1142.

1. A targeted drug conjugate composition, comprising a carrier and a precision targeted drug conjugate comprised of a therapeutic agent and a targeting agent, wherein (i) the therapeutic potency or therapeutic index of the targeted drug conjugate is at least about 10-fold more than the therapeutic potency or therapeutic index of an untargeted drug composition comprising the same therapeutic agent in non-targeted form, and (ii) the targeting agent specifically binds to an extracellular domain of a protein displayed on an outer surface of a cell membrane of a cell, and wherein optionally the therapeutic agent is conjugated to the targeting agent.

2. A targeted drug conjugate composition according to claim 1 wherein the therapeutic agent is selected from the group consisting of a small molecule, a peptide, a protein, a nucleic acid, a radionuclide, and a gene delivery vehicle.

3. A targeted drug conjugate composition according to claim 1 wherein the therapeutic agent is selected from the group consisting of chemotherapeutic agent, an immune stimulatory agent, an anti-neoplastic agent, a pro-coagulant, a toxin, an antibiotic, a hormone, an enzyme, and a lytic agent.

4. A targeted drug conjugate composition according to claim 1 wherein the targeting agent is one member of a high-affinity binding pair, optionally a molecule selected from the group consisting of an antibody, an antigen-binding antibody fragment, a receptor, a ligand-binding receptor fragment, a receptor ligand, a small molecule, and an aptamer.

5. A targeted drug conjugate composition according to claim 1 wherein the targeted drug conjugate further comprises a linker disposed between the therapeutic agent and targeting agent.

6. A targeted drug conjugate composition according to claim 1 wherein the targeted drug conjugate is a nanoparticle, optionally a nanoparticle having a mean diameter of less than about 20 nm.

7. A pharmaceutical composition, comprising a targeted drug conjugate composition according to claim 1 wherein the carrier is a pharmaceutically acceptable carrier.

8. A targeted drug conjugate composition according to claim 1 wherein the effective amount of the therapeutic agent is present in an amount that is at least about 100 times less than the effective amount of the therapeutic agent when the therapeutic agent is a non-targeted therapeutic agent.

9. A targeted drug conjugate composition according to claim 1 wherein:
the targeting agent specifically binds to an extracellular domain of a protein displayed on the outer surface of a cell membrane of a cell that is a vascular endothelial cell;
the extracellular domain of the protein is capable of mediating active transvascular pumping of the targeted drug conjugate across the cell into underlying diseased tissue; and
the extracellular domain of the protein that is displayed on the surface of the vascular endothelial cell is predominantly located in or translocated to caveolae.

10. A method of decreasing the amount of a therapeutic agent needed to effect therapy, comprising administering a targeted drug conjugate composition according to claim 1 to a subject having a disease or condition amenable to treatment by the therapeutic agent, thereby decreasing the amount of the therapeutic agent needed to treat the disease or condition, whereby the amount of the therapeutic agent administered to the subject via the targeted drug conjugate composition is at least about 10-fold less than when the therapeutic agent present in the targeted drug conjugate is administered in a non-targeted form to treat the disease or condition.

11. A method according to claim 10 wherein the amount of the therapeutic agent is at least about 100-fold less than when the therapeutic agent present in the targeted drug conjugate composition is administered in a non-targeted form to treat the disease or condition.

12. A method according to claim 10 wherein the therapeutic agent is selected from the group consisting of a small molecule, a peptide, a protein, a nucleic acid, a radionuclide, and a gene delivery vehicle, optionally a virus.

13. A method according to claim 1 wherein the targeting agent is one member of a high-affinity binding pair, optionally a molecule selected from the group consisting of an antibody, an antigen-binding antibody fragment, a receptor, a ligand-binding receptor fragment, a receptor ligand, a small molecule, and an aptamer.

14. A method according to claim 10 wherein the disease or condition is selected from the group consisting of a non-hematologic cancer, an infection, inflammation, fibrosis, acute injury, infarction, and a pathological malfunction that is not one of the foregoing.

15. A method of treating a disease or condition, comprising administering to a subject suspected of or having a disease or condition a targeted drug conjugate composition according to claim 1, thereby treating the disease or condition.

16. A method according to claim 15 wherein the subject is a human or another mammal, optionally a mammal selected from the group consisting of an bovine, canine, equine, feline, ovine, and porcine animal.

17. A method according to claim 15 wherein the disease or condition is selected from the group consisting of a non-hematologic cancer, an infection, inflammation, fibrosis, acute injury, infarction, and a pathological malfunction that is not one of the foregoing.

18. A method according to claim 17 wherein the non-hematologic cancer is a solid cancer selected from the group consisting of a sarcoma, carcinoma, lymphoma, and metastatic lesion.
